(19) United States
(12) Patent Application Publication
(10) Pub. No.: US 2021/0009551 A1
(43) Pub. Date: Jan. 14, 2021

(54) COMPOUNDS AND METHODS FOR TREATING FIBROSIS OR CANCER

(71) Applicant: THE REGENTS OF THE UNIVERSITY OF CALIFORNIA, Oakland, CA (US)

(72) Inventors: Harold A. Chapman, Mill Valley, CA (US); Ying WEI, Foster City, CA (US); Bradley J. BACKES, San Francisco, CA (US)

(21) Appl. No.: 16/305,172

(22) PCT Filed: Jun. 2, 2017
(86) PCT No.: PCT/US2017/035687

§ 371 Date: Nov. 28, 2018

Related U.S. Application Data
(60) Provisional application No. 62/344,900, filed on Jun. 2, 2016.
(60) Provisional application No. 62/469,394, filed on Mar. 9, 2017.

(51) Int. Cl. C07D 323/00; C07D 321/10; C07D 273/01

(52) U.S. Cl. CPC C07D 323/00; C07D 273/01; C07D 321/10

(57) ABSTRACT

Provided herein, inter alia, are methods and compounds for treating a pulmonary disease, fibrosis, or cancer.

### CROSS-REFERENCES TO RELATED APPLICATIONS

This application claims the benefit of U.S. Provisional Application No. 62/469,394, filed Mar. 9, 2017 and U.S. Provisional Application No. 62/344,900, filed Jun. 2, 2016, which are incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with government support under grant no. P01 HL108794 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 48536-583001WO_ST25.TXT, created on May 31, 2017, 6,316 bytes, machine format IBM-PC, MS-Windows operating system, is hereby incorporated by reference.

### BACKGROUND

Tissue fibrosis is a major cause of human morbidity and mortality worldwide^{1,8}. TGFβ1 signaling through its heterodimeric receptor is a well-known driver of collagen expression and tissue accumulation important to wound repair⁹. TGFβ1 signaling is also strongly implicated in numerous fibrotic diseases including liver, heart, and lung fibrosis as well as cancer progression¹⁰⁻¹³. Although attractive as a therapeutic target, the critical roles of TGFβ1 in suppressing inflammation and epithelial proliferation give pause to the idea of global inhibition². Indeed systemic inhibition of TGFβ1 leads to the development of squamous skin tumors and altered immunity^{14,15}.

Idiopathic Pulmonary Fibrosis (IPF) is a chronic progressive interstitial lung disease with increasing prevalence, high mortality rates and poor treatment options. Greater than 50 percent of IPF patients die within three years of diagnosis and the only curative treatment is lung transplantation. Drugs in clinical trial to treat IPF show limited efficacy. There is a pressing need for new therapies for IPF. In addition, tumor cell stiffness as a consequence of cross-linked collagen accumulation has emerged as an important risk factor for metastatic disease. Lysyl oxidase (LOX) enzymes are strongly implicated in tumor collagen cross-linking. There is unmet medical need in the area of cancer metastasis. Disclosed herein, inter alia, are solutions to these and other problems in the art.

### BRIEF SUMMARY OF THE INVENTION

In an aspect is provided a compound having the formula:

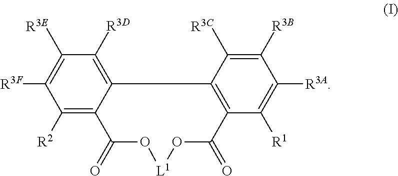

R¹ is independently hydrogen, halogen, —CX¹₃, —CHX¹₂, —CH₂X¹, —OCX¹₃, —OCH₂X¹, —OCHX¹₂, —CN, —N₃, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is independently hydrogen, halogen, —CX²₃, —CHX²₂, —CH₂X², —OCX²₃, —OCH₂X², —OCHX²₂, —CN, —N₃, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹ is a substituted or unsubstituted alkylene, substituted or unsubstituted heteroalkyl ene, substituted or unsubstituted cycloalkylene, substituted or unsubstituted heterocycloalkyl ene, substituted or unsubstituted aryl ene, or substituted or unsubstituted heteroarylene. R^{1A}, R^{1B}, R^{1C}, R^{1D}, R^{2A}, R^{2B}, R^{2C}, and R^{2D} are independently hydrogen, —CX₃, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{3A}, R^{3B}, R^{3C}, R^{3D}, R^{3E}, and R^{3F} are independently hydrogen, —CX³₃, —CN, —N₃, —OH, —COOH, —CONH₂, —NH₂, substituted or unsubstituted alkyl, substituted or unsubstituted heteroalkyl (e.g., substituted or unsubstituted alkoxy), substituted or unsubstituted cycloalkyl, substituted or unsubstituted heterocycloalkyl, substituted or unsubstituted aryl, substituted or unsubstituted heteroaryl, —O-(substituted or unsubstituted alkyl), —O-(substituted or unsubstituted heteroalkyl), —O-(substituted or unsubstituted cycloalkyl), O(substituted or unsubstituted heterocycloalkyl), O(substituted or unsubstituted aryl), or O(substituted or unsubstituted heteroaryl). R^{1A} and R^{1B} substituents bonded to the same nitrogen atom may optionally be joined to form a substituted or unsubstituted heterocycloalkyl or substituted or unsubstituted heteroaryl. R^{2A} and R^{2B} substituents bonded to the same nitrogen atom may optionally be joined to form a substituted or unsubstituted heterocycloalkyl or substituted or unsubstituted heteroaryl. Each X, X¹, X², and X³ is independently —F, —Cl, —Br, or —I.

In an aspect is provided a pharmaceutical composition including a compound described herein, or pharmaceutically acceptable salt thereof, and a pharmaceutically acceptable excipient.

In an aspect is provided a method of treating fibrosis including administering to a subject in need thereof an effective amount of a compound described herein.

In an aspect is provided a method of inhibiting Lysyl oxidase homolog 2 protein activity including contacting the Lysyl oxidase homolog 2 protein with a compound described herein.

In an aspect is provided a method of inhibiting SNAIL1 protein activity including contacting the SNAIL1 protein with a compound described herein.

In an aspect is provided a method of reducing the level of activity of zinc finger protein Snail1 in a subject, the method including administering an effective amount of a compound described herein to the subject.

In an aspect is provided a method of reducing the level of activity of Lysyl oxidase homolog 2 in a subject, the method including administering an effective amount of a compound described herein to the subject.

In an aspect is provided a method of inhibiting Transforming growth factor beta 1 (TGF-β1) protein activity including contacting the TGF-β1 protein with a compound described herein.

In an aspect is provided a method of inhibiting Transforming growth factor beta 1 (TGF-β1) signal transduction pathway activity of a cell including contacting the cell with a compound described herein.

In an aspect is provided a method of inhibiting collagen cross-linking in a subject, the method including administering an effective amount of a compound described herein to the subject.

In an aspect is provided a method of treating fibrosis in a subject, the method including administering an effective amount of a compound described herein to the subject.

In an aspect is provided a method of treating pulmonary fibrosis in a subject, the method including administering an effective amount of a compound described herein to the subject.

In an aspect is provided a method of treating idiopathic pulmonary fibrosis in a subject, the method including administering an effective amount of a compound described herein to the subject.

In an aspect is provided a method of treating cancer in a subject, the method including administering an effective amount of a compound described herein to the subject.

In an aspect is provided a method of treating cancer metastasis in a subject, the method including administering an effective amount of a compound described herein to the subject.

In an aspect is provided a method of inhibiting (e.g., reducing compared to control, reducing compared to absence of the compound) LOXL2 protein activity and Transforming growth factor beta Receptor 1 (TGFβRI) protein activity in a cell, including contacting the LOXL2 protein with a LOXL2 generated TGFβRI inhibitor precursor (e.g., LOXL2 generated TGFβRI inhibitor compound precursor), wherein the LOXL2 protein modifies the LOXL2 generated TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound); and wherein the LOXL2 generated TGFβRI inhibitor (e.g., LOXL2 generated TGFβRI inhibitor compound) contacts a TGFβRI protein.

In an aspect is provided a method of detecting inhibition of fibrosis, the method including 1) administering a compound described herein to a subject having fibrosis; 2) measuring the level of pyridinoline (PYD) and/or deoxypyridinoline in a biological sample (e.g., blood or urine of the subject); and detecting the presence of inhibition of fibrosis by detecting a reduction in the level of pyridinoline (PYD) and/or deoxypyridinoline in the biological sample (e.g., blood or urine of the subject).

### BRIEF DESCRIPTION OF THE DRAWINGS

FIGS. 1A-1N. Ellagic acid and corilagin inhibit TGFβ1-dependent EMT and red raspberry diet attenuates bleomycin-induced fibrogenesis and blocks lung tumor metastasis. FIG. 1A, Structure of ellagic acid. FIG. 1B, Immunofluorescence imaging of A549 cells±TGFβ1 for 48 h in the presence of DMSO, SB431542 (SB), or ellagic acid (EA). E-cadherin green, fibronectin orange, and DAPI blue. Scale bar, 500 μm. FIG. 1C, A549 cells were treated with ellagic acid (1 μM) and stimulated with TGFβ1 for 1.5 h and the lysates were blotted for p-smad2 and smad2. β-Actin is used as loading control. FIG. 1D, Hydroxyproline analysis of lung tissues from ctl or EA chow-treated mice 21 days after bleomycin (n=10 female mice/group) or saline instillation (n=4 female mice/group). Data are expressed as mean±SEM. P value by unpaired two-tailed t-test. FIG. 1E, Masson's Trichrome staining of lung sections from ctl or EA pump-treated mice 17 days after bleomycin or saline instillation. Mosaic images (4×) covering whole lung section were shown. FIG. 1F, Structure of corilagin. FIG. 1G, A549 cells stimulated with TGFβ1 were treated with corilagin (0-5 μM) for 48 h and the lysates were blotted for fibronectin, E-cadherin, and snail 1. β-actin is used as loading control. FIG. 1H, Hydroxyproline analysis of lung tissues from vehicle or corilagin-gavage treated mice 21 days after bleomycin (n=9 female mice/group) or saline instillation (n=7 female mice/group). Data are expressed as Mean±SEM. P value by unpaired two-tailed t-test. FIG. 1I, Pro-fibrotic markers measured in protein extracts from snap-frozen mouse lungs intratracheally given saline or bleomycin and orally treated with vehicle or corilagin. Tissue lysates were blotted for fibronectin, collagen I, snail 1, β-actin, p-smad3, and total smad3. FIG. 1J, Dosing and treatment in lung fibrosis model. Mice were injected intratracheally with bleomycin (1.9 units/kg). The red raspberry diet (EA Chow) and control diet (Ctl Chow) were given to mice for 21 days. Osmotic pumps loaded with EA salt or PBS were implanted on mice at D10 after bleomycin for 7 days. Vehicle or corilagin were given to mice by daily gavage starting from D10 after bleomycin for 11 days. FIG. 1K, Implantation and treatment in syngeneic lung cancer model. Metastatic 344SQ tumor cells were subcutaneously injected in syngeneic mice at 12-week old and treated with ctl or EA chow for 5 weeks. FIG. 1L, Total tumor weight (grams) of ctl or EA chow treated 344SQ primary tumors (n=14 per group). FIG. 1M, Lung metastasis of 344SQ tumors treated with ctl or EA chow (n=14 per group). FIG. 1N, Ctl or EA chow treated 344SQ primary tumors were lysed and blotted for fibronectin, collagen I, β-actin, p-smad3, and total smad3.

FIGS. 2A-2I Ellagic acid and corilagin inhibit LOXL2-dependent collagen cross-linking and TGFβ1 response. FIG. 2A, LTQ cycle. LTQ converts lysine to allysine and yields an aminophenol intermediate. Subsequent hydrolyses releases allysine and the original cofactor, producing hydrogen peroxide and ammonia as side products. FIG. 2B, Primary human lung fibroblasts cultured in the presence of Vitamin C and Dextran Sulfate were treated with recombinant human LOXL2 (LOXL2) in addition to different inhibitors for 7 days. The insoluble cross-linked collagen was extracted and measured by Sircol assay. SB: SB431542, Alk5 inhibitor; NAC: N-Acetyl Cysteine, antioxidant. FIG. 2C, Recombinant human LOXL2 was incubated with 2 mM penicilamine (DPA) or different concentrations of corilagin (0-1 μM) for 1 h and LOX activity was measured. FIG. 2D, NMuMG cells over-expressing human LOXL2 incubated with or without 0.5 μM corilagin for 24 h were lysed and blotted for LOXL2, snail 1, and β-actin. FIG. 2E, Collagen cross-links in 344SQ primary tumors treated with ctl or EA chow. FIG. 2F, Mouse pyridinoline (PYD) and deoxypyridinoline (DPD) were measured at different time points (D0-D21) in the urine samples pool-collected from mice injected with bleomycin and treated with vehicle (n=5) or corilagin (n=5). Three urine sample collections at each time point. P value by unpaired two-tailed t-test. FIG. 2G, Human PYD and DPD were measured in the urine samples collected independently from two cohorts of healthy donors and IPF patients from University of California at San Francisco (UCSF) and University of Texas Health Science Center at San Antonio (San Antonio). The data was analyzed by Mann-Whitney test. The unit for DPD and PYD is nmol/mmol creatinine. FIG. 2H, A549 cells transfected with siRNA to LOXL2 were stimulated with TGFβ1 or left un-stimulated for 48 h in the presence or absence of 1 μM corilagin. The lysates were blotted for LOXL2, fibronectin, E-cadherin, Snail1, and β-actin. FIG. 2I, Primary human lung fibroblasts transfected with siRNAs to LOXL1 or LOXL2 were stimulated with TGFβ1 or left un-stimulated for 72 h in the presence or absence of 1 μM corilagin and the lysates were blotted for LOXL1, LOXL2, N-cadherin, alpha smooth muscle actin (α-SMA), snail 1, and β-actin.

FIGS. 3A-3F Corilagin inhibition of TGFβ1 signaling is dependent on LOXL2 activity. FIG. 3A, A549 cells pre-treated with 1 μM corilagin for 0 h, 3 h, or 6 h were stimulated with different doses of TGFβ1 (0, 0.2, or 1 ng/ml) for 30 min and the cell lysates were blotted for p-smad2, p-smad3, smad2, smad3, and β-actin. FIG. 3B, NMuMG cells transfected with human LOXL2 or empty vector were pretreated with 1 μM corilagin or DMSO for 6 h and then incubated without or with TGFβ1 for 30 min. The cell lysates were blotted for LOXL2, p-smad3, smad3, and β-actin. FIG. 3C-FIG. 3D, A549 cells transfected with siRNA to LOXL2 (FIG. 3C) and primary human lung fibroblasts transfected with siRNAs to LOXL1 or LOXL2 (FIG. 3D) were pre-treated with 1 μM corilagin or DMSO for 6 h before incubating without or with TGFβ1 for 30 min. The cell lysates were blotted for p-smad3 and smad3. Ratio of p-smad3/smad3 for each lane was shown. FIG. 3E, Correlation of cellular LOXL2 levels and corilagin inhibition of TGFβ1 signaling. FIG. 3F, A549 cells were pre-treated with 1 μM corilagin with or without 2 mM penicilamine (DPA) for 6 h before TGFβ1 stimulation for 30 min. The cell lysates were blotted for p-smad3, smad3, and β-actin.

FIGS. 4A-4K Trihydroxyphenolic motif binds and inhibits LOXL2 activity and generates a novel TGFβ1 receptor kinase inhibitor. FIG. 4A, Catalytic domain sequence of LOXL2 (SEQ ID NO: 1). Catalytic domain: P548-S751. LTQ sites are highlighted and bolded (K653 and Y689). The three point mutation sites are in red and bolded (K614N, K731R, K759R). FIG. 4B, NMuMG cells transiently transfected with wild-type or mutant human LOXL2 were treated with 1 μM corilagin or DMSO for 6 h. LOX activity of conditioned media from treated cells was measured. Data presented as percent inhibition by corilagin. FIG. 4C, NMuMG cells transfected with wild-type or mutant human LOXL2 were pretreated with 1 μM corilagin or DMSO for 6 h and then incubated without or with TGFβ1 for 30 min. The cell lysates were blotted for LOXL2, p-smad3, smad3, and β-actin. FIG. 4D, NMuMG cells transfected with wild-type or mutant human LOXL2 were incubated with or without 1 μM corilagin for 24 h and lysates were blotted for LOXL2, snail 1, and β-actin. FIG. 4E, NMuMG cells transfected with wild-type or mutant human LOXL2 incubated with 1 μM corilagin for 6 h and labelled with 2.5 mM biotin-hydrazide for 2 h. The biotin-hydrazide linked carbonylated LOXL2 was pulled down with streptavidin-magnetic beads and the precipitates and input protein were blotted for LOXL2. FIG. 4F. Structure 3Abd and derivatives. FIG. 4G, A549 cells were stimulated with TGFβ1 for 30 min in the presence or absence of catechol compounds (0.5-10 μM) without pre-incubation. Cell lysates were blotted for p-smad3, smad3, and β-actin. FIG. 4H, A549 cells were stimulated with TGFβ1 or left un-stimulated for 48 h in the presence or absence of 3Abd (0.5-10 μM). The lysates were blotted for LOXL2, fibronectin, E-cadherin, snail 1, and β-actin. FIG. 4I, Purified ALK5/TGFβRI catalytic domain kinase assay was carried out in the presence of ten doses of 3Abd or 2Abd starting from 100 μM. The kinase activity was indicated by ³³P-ATP signals and the IC50 of 3 Abd was calculated as ˜3 μM. FIG. 4J, NMuMG and A549 cells transiently transfected with SEE reporter pGL(CAGA)₁₂Luc were seeded into 96-well plate 24 hours after transfection. Co-cultured wells were seeded with 5K transfected NMuMG cells and 25K non-transfected A549 cells. The cells were pretreated with or without 1 μM corilagin for 6 hours and stimulating with TGFβ1 overnight before lysis for luciferase assay. FIG. 4K, A schematic illustrates mechanism by which trihydroxyphenols inhibit LOXL2 and TGFβ1 signaling. (1) Trihydroxyphenolic unit binds Lys731 in LOXL2 catalytic domain and inactivates enzyme activity, therefore blocks collagen cross-linking and snail 1 protein stabilization; (2) LOXL2 metabolizes hydroxyphenolic unit to generate active inhibitor to TGFβ1 signaling, blocking collagen accumulation.

FIGS. 5A-5H Ellagic acid and catechins inhibit TGFβ1-dependent EMT and red raspberry diet decreases lung tumor fibrillar collagen. FIG. 5A, A549 cells were stimulated with TGFβ1 in the presence of ALK5 inhibitor SB431542 and 19 lead compounds from high-throughput screening for 48 hours. The lysates were blotted for fibronectin, E-cadherin, and β-actin. The cells treated for 2 hours were blotted for p-smad2 and smad2. FIG. 5B, The survival of bleomycin-treated mice by day 21 of EA chow and day 17 of EA pump treatment were plotted and analyzed by Chi test. FIG. 5C, Plasma levels of corilagin 2 hours after the last oral dosing of 100 mg/kg at day 21. n=5. FIG. 5D, Primary subcutaneous tumor volume of 344SQ tumor cells injected in syngeneic mice treated with ctl or EA chow (n=14 per group). FIG. 5E, Quantification of curvature ratio for individual collagen fibers imaged by SHG microscopy of primary 344SQ tumor tissues treated with ctl (n=102 collagen fibers per sample) or EA chow (n=141 collagen fibers per sample). FIG. 5F, A549 cells stimulated with TGFβ1 were treated with different catechins (1 and 10 μM) for 48 h and the lysates were blotted for fibronectin, E-cadherin, snail 1, and β-actin. FIG. 5G, Correlation of trihydroxyphenolic motif and inhibition of TGFβ1-induced snail 1. FIG. 5H, Structure comparison of corilagin, catechins, and luteolin.

FIGS. 6A-6H Ellagic acid and corilagin do not affect immune cell distribution or macrophage TGFβ1 response in vivo, a-c, Total protein concentration (FIG. 6A), total cell counts (FIG. 6B), and immune cell distribution (FIG. 6C) from bronchoalveolar lavage fluid (BALF) of mice intratracheal injected with saline or bleomycin treated with ctl or EA chow. FIG. 6D-FIG. 6F, Total protein concentration (FIG. 6D), total cell counts (FIG. 6E), and immune cell distribution (FIG. 6F) from BALF of mice intratracheal injected with saline or bleomycin treated with vehicle or corilagin. FIG. 6G, Transcripts of TGFβ1-responsive genes were measured in RNA extracts by qRT-PCR from BALF pellets intratracheal injected with saline or bleomycin for 21 days and treated with EA or control pump (n=3-5/group). Metalloproteinase (MMP)9, MMP12, TREM1, and plasminogen activator type I (PAI)1 mRNA levels are normalized to that of β-actin. FIG. 6H, Airway macrophages from BALF of mice intratracheal injected with saline or bleomycin for 5 days and treated with vehicle or corilagin were lysed and blotted for p-smad3 and β-actin (left). Quantification (mean±SEM) of densitometry values of p-smad3/β-actin ratio (n=6) was shown (right). P value by unpaired two-tailed t-test. “ns”, not significant by t-test.

FIGS. 7A-7F Trihydroxyphenolic compounds inhibit LOXL2-dependent collagen cross-linking and long-term EA chow treatment does not affect bone mineral density or aorta collagen content. FIG. 7A-FIG. 7B, Primary human lung fibroblasts cultured in the presence of Vitamin C and Dextran Sulfate were treated with recombinant human LOXL2 (rhLOXL2) in the presence of different concentrations of EA or corilagin (0-500 nM) (FIG. 7A) or catechins (FIG. 7B) for 7 day. The insoluble cross-linked collagen were extracted and measured by Sircol assay. FIG. 1C, Correlation of trihydroxyphenolic motif and inhibition of rhLOXL2-induced collagen cross-linking. FIG. 7D, Change in bone mineral density (% BMD Change Over 6 Months) from baseline at lumbar spine and proximal femur in mice treated with ctl chow (n=8) or EA chow (n=8). Data are expressed as mean±SEM. “ns” indicates not significant (Student's t-test). FIG. 7E, Hydroxyproline analysis of aortas isolated from mice treated with ctl chow (n=4) or EA chow (n=4) for 6 months. Data are expressed as mean±SEM. “ns” indicates not significant (Student's t-test). FIG. 7F, Elastic van Gieson stain of paraffin sections of aortas from mice treated with ctl chow (n=4) or EA chow (n=4) for 6 months.

FIGS. 8A-8G Trihydroxyphenolic compounds inhibit TGFβ1 signaling and corilagin inhibition of TGFβ1 responses is dependent on LOXL2 activity. FIG. 7A, A549 cells pre-treated with different inhibitors for 6 h were stimulated with TGFβ1 (4 ng/ml) for 30 min and the cell lysates were blotted for p-smad3, smad3, and β-actin. FIG. 7B, A549 cells pre-treated with corilagin and catechins for 6 h were stimulated with TGFβ1 for 30 min and the cell lysates were blotted for p-smad3 and smad3. FIG. 1C, Correlation of trihydroxyphenolic motif and inhibition of TGFβ1 signaling. FIG. 7D, A549 and MDA-MB-231 cells were pre-treated with 1 μM corilagin for 6 h before stimulating with EGF (50 ng/ml) for 5 min or with TGFβ1 for 30 min and the cell lysates were blotted for p-EGFR and total EGFR or p-smad3 and smad3, respectively. FIG. 7E, Primary human lung fibroblasts were pre-treated with 1 μM corilagin with or without 2 mM penicilamine (DPA) for 6 h before TGFβ1 stimulation for 30 min. The cell lysates were blotted for p-smad3, smad3, and β-actin. FIG. 7F, A549 cells were stimulated with TGFβ1 or left un-stimulated for 48 h in the presence or absence of 1 μM corilagin with or without 2 mM penicilamine (DPA) and the lysates were blotted for fibronectin, E-cadherin, snail 1, and β-actin. FIG. 7G, Primary human lung fibroblasts were stimulated with TGFβ1 in the presence or absence of 1 μM corilagin with or without penicillamine (DPA) for 72 h. The lysates were blotted for fibronectin, collagen I, N-cadherin, α-SMA, snail 1, and β-actin.

FIGS. 9A-9C Corilagin metabolizes in LOXL2-high A549 culture medium but not in LOXL2-low HaCaT culture medium. FIG. 9A, LOXL2-high A549 and LOXL2-low HaCaT cells were treated with 1 μM corilagin and the conditioned medium was collected at 0, 0.5, and 5 hrs and corilagin levels were measured by LC-MS. Corilagin concentration decreased dramatically in A549 cells at 5 hrs but not in HaCaT cells. FIG. 9B, A549 cells were treated 1 μM corilagin with or without 2 mM LOXL2 inhibitor peniciliamine (DPA) for hrs and the corilagin metabolite Ml was detected by LC-MS/MS. The generation of Ml was inhibited by blocking LOXL2 activity with DPA. FIG. 9C, LC-MS/MS analyses suggested that Ml is like to be derived from corilagin and may contain a nitrogen.

FIGS. 10A-10C Trihydroxyphenolic motif binds and inhibits LOXL2 activity and generates novel specific TGFβ1 receptor kinase inhibitor. FIG. 10A, NMuMG cells transiently transfected with wild-type or mutant human LOXL2 were treated with 1 μM corilagin or DMSO for 6 h. LOX activity of conditioned media from treated cells was measured. Data presented as relative intensity at Ex/Em 540/590 nm. FIG. 10B, MCF-10A and NMuMG cells were stimulated with TGFβ1 for 30 min in the presence or absence of 3Abd (10 and 20 μM) without pre-incubation. Cell lysates were blotted for p-smad3, smad3, and β-actin. FIG. 10C, A549 cells transfected with Flag-tagged TRI and TRII were immunoprecipitated with anti-Flag antibody and the in vitro kinase assay was performed on beads in the presence or absence of 20 μM 3Abd or 3 Cc. The final reaction was eluted and analyzed by blotting for phosphotyrosine and Flag.

FIGS. 11A-11F Novel chemical structures, compound A (FIG. 11A), compound B (FIG. 11B), compound C (FIG. 11C), compound D (FIG. 11D), compound E (FIG. 11E), compound F (FIG. 11F), compound G (FIG. 11G), compound H (FIG. 11H), compound I (FIG. 11I). See Example 10 for synthetic and characterization details.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, P, Si, or S) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridy 1), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroaryl ene refers to two rings fused together, wherein one ring has 6 members and the other ring has 6 members, and wherein at least one ring is a heteroaryl ring. And a 6,5-fused ring heteroaryl ene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkyl aryl ene” as an aryl ene moiety covalently bonded to an alkyl ene moiety (also referred to herein as an alkylene linker). In embodiments, the alkylarylene group has the formula:

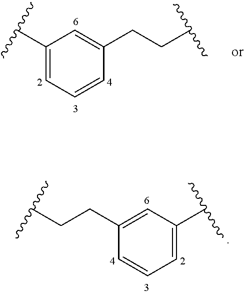 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6), for example with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl. In embodiments, the alkylarylene is unsubstituted.

Each of the above terms (e.g., “alkyl,” “heteroalkyl,” “cyclalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″′, —NR—C(NR′R″)═NR′″, —S(O)R′, —S(O)₂R′, —S(O)₂NR′R″, —NRSO₂R, —NR′NR′R′″, —ONR′R″, —NR′C(O)NR″NR″′R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″′ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″′ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″′, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR″′R″′, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″′ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo,
halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to membered, 5 to 9 membered, or 5 to 6 membered), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo,
   halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂N H₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
   (ii) alkyl (e.g., C₁-C₂₀, C₁-C₁₂, C₁-C₈, C₁-C₆, C₁-C₄, or C₁-C₂), heteroalkyl (e.g., 2 to membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, to 9 membered, or 5 to 6 membered), substituted with at least one substituent selected from:
      (a) oxo,
      halogen, —CCl₃, —CBr₃, —CF₃, —CI₃, —CHCl₂, —CHBr₂, —CHF₂, —CHI₂, —CH₂Cl, —CH₂B r, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
      —NHC(O)NH₂, —NHSO₂H, —NHC(O)H,
      —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, unsubstituted alkyl (e.g., C₁-C₂₀, C₁-C₁₂, C₁-C₈, C₁-C₆, C₃-C₄, or C₃-C₂), unsubstituted heteroalkyl (e.g., 2 to membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
      (b) alkyl (e.g., C₁-C₂₀, C₁-C₁₂, C₁-C₈, C₁-C₆, C₃-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo,
      halogen, —CCl₃, —CBr₃, —CF₃, —CI₃, —CHCl₂, —CHBr₂, —CHF₂, —CHI₂, —CH₂Cl, —CH₂B r, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,
      —NHC(O)NH₂, —NHSO₂H, —NHC(O)H,
      —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂F, —OCH₂I, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkyl ene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkyl ene, each substituted or unsubstituted arylene is a substituted or unsubstituted C₆-C₁₀ aryl ene, and/or each substituted or unsubstituted heteroaryl ene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkyl ene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating” and the like in reference to a protein refers to conversion of a protein into a biologically active derivative from an initial inactive or deactivated state. The terms may reference activation, or activating, sensitizing, or up-regulating signal transduction or enzymatic activity or the amount of a protein decreased in a disease.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, in embodiments, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The term “Lysyl oxidate homolog 2” or “LOXL2” refers to a protein (including homologs, isoforms, and functional fragments thereof). In embodiments, the LOXL2 protein encoded by the LOXL2 gene has the amino acid sequence set forth in or corresponding to Entrez 4017, UniProt Q9Y4K0, or RefSeq (protein) NP_002309. In embodiments, the LOXL2 gene has the nucleic acid sequence set forth in RefSeq (mRNA) NM_002318. In embodiments, the amino acid sequence or nucleic acid sequence is the sequence known at the time of filing of the present application. In embodiments, the sequence corresponds to 01:4505011. In embodiments, the sequence corresponds to NP_002309.1. In embodiments, the sequence corresponds to NM_002318.2. In embodiments, the sequence corresponds to 01:67782347. In embodiments, the LOXL2 is a human LOXL2.

The term “zinc finger protein SNAI1” or “SNAIL1” or “SNAI1” refers to a protein (including homologs, isoforms, and functional fragments thereof). In embodiments, the SNAIL1 protein encoded by the SNAIL1 gene has the amino acid sequence set forth in or corresponding to Entrez 6615, UniProt 095863, or RefSeq (protein) NP_005976. In embodiments, the SNAIL1 gene has the nucleic acid sequence set forth in RefSeq (mRNA) NM_005985. In embodiments, the amino acid sequence or nucleic acid sequence is the sequence known at the time of filing of the present application. In embodiments, the sequence corresponds to GI: 18765741. In embodiments, the sequence corresponds to NP_005976.2. In embodiments, the sequence corresponds to NM_005985.3. In embodiments, the sequence corresponds to GL301336132. In embodiments, the SNAIL1 is a human SNAIL1.

The term “TGFβ-1” refers to a transforming growth factor beta 1 (including homologs, isoforms, and functional fragments thereof) which is involved in cellular growth and proliferation. The term includes any recombinant or naturally-occurring form of TGFβ-1, or variants thereof, that maintain TGFβ-1 activity (e.g. within at least 30%, 40%, 50%, 60%, 70%, 80%, 90%, 95%, or 100% activity compared to wildtype TGFβ-1). In embodiments, the TGFβ-1 protein encoded by the TGFβ-1 gene has the amino acid sequence set forth in or corresponding to Entrez 7040, UniProt P01137, RefSeq NM_000660, or RefSeq NP_000651. In embodiments, the TGFβ-1 gene has the nucleic acid sequence set forth in RefSeq (mRNA) NM_000660.5. In embodiments, the amino acid sequence or nucleic acid sequence is the sequence known at the time of filing of the present application. In embodiments, the sequence corresponds to GI: 551411950. In embodiments, the sequence corresponds to NP_000651.3. In embodiments, the sequence corresponds to GI: 63025222. In embodiments, the TGFβ-1 is a human TGFβ-1, such as a human cancer causing TGFβ-1.

The term “TGFβRI” refers to a transforming growth factor beta Receptor 1 (including homologs, isoforms, and functional fragments thereof) which is a serine/threonine protein kinase and may form heteromeric complexes with type II TGFβ Receptors when binding to TGF-β. The term includes any recombinant or naturally-occurring form of TGFβRI, or variants thereof, that maintain TGFβRI activity (e.g. within at least 30%, 40%, 50%, 60%, 70%, 80%, 90%, 95%, or 100% activity compared to wildtype TGFβRI). In embodiments, the TGFβRI protein encoded by the TGFβRI gene has the amino acid sequence set forth in or corresponding to Entrez 7046, UniProt P36897, RefSeq NM_001306210, or RefSeq NP_001293139. In embodiments, the TGFβRI gene has the nucleic acid sequence set forth in RefSeq (mRNA) NM_001306210.1. In embodiments, the amino acid sequence or nucleic acid sequence is the sequence known at the time of filing of the present application. In embodiments, the sequence corresponds to NP_001293139.1. In embodiments, the TGFβRI protein encoded by the TGFβRI gene has the amino acid sequence set forth in or corresponding to Entrez 7046, UniProt P36897, RefSeq NM_004612, or RefSeq NP_004603. In embodiments, the TGFβRI gene has the nucleic acid sequence set forth in RefSeq (mRNA) NM_004612.3. In embodiments, the amino acid sequence or nucleic acid sequence is the sequence known at the time of filing of the present application. In embodiments, the sequence corresponds to NP_004603.1. In embodiments, the TGFβRI is a human TGFβRI, such as a human cancer causing or fibrosis causing TGFβRI.

A “transforming growth factor beta Receptor 1 inhibitor” or “TGFβRI inhibitor” is a composition (e.g., a biomolecule) that decreases the activity or function of TGFβRI relative to the activity or function of TGFβRI in the absence of the inhibitor (e.g., wherein the TGFβRI inhibitor binds TGFβRI). In embodiments, the TGFβRI inhibitor is not a protein. A “transforming growth factor beta Receptor 1 inhibitor compound” or “TGFβRI inhibitor compound” refers to a compound (e.g. compounds described herein) that decreases the activity of TGFβRI relative to the activity of TGFβRI in the absence of the inhibitor.

A “LOXL2 generated transforming growth factor beta Receptor 1 inhibitor” or “LOXL2 generated TGFβRI inhibitor” is a TGFβRI inhibitor (e.g., compound) that is a product of a LOXL2 protein reaction (e.g., replacement of a composition —OH with a —NH₂). A “LOXL2 generated transforming growth factor beta Receptor 1 inhibitor compound” or “LOXL2 generated TGFβRI inhibitor compound” refers to a compound (e.g. compounds described herein) that is a product of a LOXL2 protein reaction (e.g., replacement of a composition —OH with a —NH₂). A LOXL2 generated TGFβRI inhibitor (e.g., LOXL2 generated TGFβRI inhibitor compound) is formed by the activity of a LOXL2 protein from the reactant “LOXL2 generated transforming growth factor beta Receptor 1 inhibitor precursor” or “LOXL2 generated TGFβRI inhibitor precursor” (e.g., LOXL2 generated TGFβRI inhibitor compound precursor). In embodiments, the LOXL2 generated TGFβRI inhibitor precursor (e.g., LOXL2 generated TGFβRI inhibitor compound precursor) includes a trihydroxyphenol moiety. In embodiments, the LOXL2 generated TGFβRI inhibitor (e.g., LOXL2 generated TGFβRI inhibitor compound) includes a 1-amino-2,3-dihydroxyphenol moiety. In embodiments, the LOXL2 generated TGFβRI inhibitor precursor (e.g., LOXL2 generated TGFβRI inhibitor compound precursor) is a LOXL2 inhibitor (e.g., inhibits LOXL2 during or after transformation of the LOXL2 generated TGFβRI inhibitor precursor to a LOXL2 generated TGFβRI inhibitor by LOXL2).

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be fibrosis. The disease may be pulmonary fibrosis. The disease may be idiopathic pulmonary fibrosis. The disease may be cancer. The disease may be a fibrotic pulmonary disease. The disease may be acute lung injury. The disease may be fibrosis. The disease may be a cancer. The disease may be a pulmonary fibrotic condition. The disease may be a pulmonary disease. The disease may be cirrhosis.

The pulmonary diseases contemplated herein can include any pulmonary disorders, lung fibrosis diseases, interstitial lung diseases, idiopathic interstitial pneumonias (IIP), idiopathic pulmonary fibrosis, familial interstitial pneumonia (FIP), non-specific interstitial pneumonia (NSIP), hypersensitivity pneumonitis, acute respiratory distress syndrome (ARDS), scleroderma associated interstitial lung disease (SSc-ILD), Sarcoidosis, Beryllium disease, rheumatoid arthritis associated lung disorder, collagen vascular associated lung disorder, cigarette smoke associated lung disorders, Sjögren's syndrome, mixed connective tissue disease, etc.

Pulmonary fibrotic conditions, e.g., interstitial lung diseases (ILD), are characterized by shortness of breath, chronic coughing, fatigue and weakness, loss of appetite, and rapid weight loss. Pulmonary fibrosis is commonly linked to interstitial lung diseases (e.g., autoimmune disorders, viral infections or other microscopic injuries), but can be idiopathic. Fibrosis involves exchange of normal lung tissue with fibrotic tissue (scar tissue) that leads to reduced oxygen capacity.

Idiopathic interstitial pneumonias (IIP) are a subset of diffuse interstitial lung diseases of unknown etiology (the term “idiopathic” indicates unknown origin). IIPs are characterized by expansion of the interstitial compartment (i.e., that portion of the lung parenchyma sandwiched between the epithelial and endothelial basement membranes) with an infiltrate of inflammatory cells. The inflammatory infiltrate is sometimes accompanied by fibrosis, either in the form of abnormal collagen deposition or proliferation of fibroblasts capable of collagen synthesis.

Idiopathic Pulmonary Fibrosis (IPF) occurs in thousands of people worldwide with a doubling of prevalence over the past 10 years. Onset of IPF may occur around 50 to 70 years of age and may start with progressive shortness of breath and hypoxemia. IPF median survival is around 3-5 years and is to date untreatable. About 5-20 percent of all cases of IPF have a family history and inheritance appears to be autosomal dominant. The clinical course of IPF is highly variable. Individuals diagnosed with IPF can experience a slow and steady decline, whereas others may decline more rapidly, thereby exhibiting a progressive form of idiopathic pulmonary fibrosis. Patients may also experience periods of relative stability interrupted by periods of rapid decline (known as acute exacerbations). A progressive form of idiopathic pulmonary fibrosis is characterized by the rapid decline of clinical and physical markers (e.g., breathing metrics, such as forced expiratory volume (FEV1), vital capacity (VC), forced vital capacity (FVC), or FEV1/FVC and diffusing capacity of carbon monoxide (DL_{CO}).

Additional fibrotic pulmonary diseases include Acute Interstitial Pneumonia (AIP), Respiratory Bronchiolitis-associated Interstitial Lung Disease (RBILD), Desquamative Interstitial Pneumonia (DIP), Non-Specific Interstitial Pneumonia (NSIP), and Bronchiolitis obliterans, with Organizing Pneumonia (BOOP).

AIP is a rapidly progressive and histologically distinct form of interstitial pneumonia. The pathological pattern is an organizing form of diffuse alveolar damage (DAD) that is also found in acute respiratory distress syndrome (ARDS) and other acute interstitial pneumonias of known causes (see Clinical Atlas of Interstitial Lung Disease (2006 ed.) pp 61-63).

RBILD is characterized by inflammatory lesions of the respiratory bronchioles in cigarette smokers. The histologic appearance of RBILD is characterized by the accumulation of pigmented macrophages within the respiratory bronchioles and the surrounding airspaces, variably, peribronchial fibrotic alveolar septal thickening, and minimal associated mural inflammation (see Wells et al. (2003) Sem Respir. Crit. Care Med. vol. 24).

DIP is a rare interstitial lung disease characterized by the accumulation of macrophages in large numbers in the alveolar spaces associated with interstitial inflammation and/or fibrosis. The macrophages frequently contain light brown pigment. Lymphoid nodules are common, as is a sparse but distinct eosinophil infiltrate. DIP is most common in smokers (see Tazelaar et al. (Sep. 21, 2010) Histopathology).

NSIP is characterized pathologically by uniform interstitial inflammation and fibrosis appearing over a short period of time. NSIP differs from other interstitial lung diseases in that it has a generally good prognosis. In addition, the temporal uniformity of the parenchymal changes seen in NSIP contrasts greatly with the temporal heterogeneity of usual interstitial pneumonia (see Coche et al. (2001) Brit J Radiol 74:189).

BOOP, unlike NSIP, can be fatal within days of first acute symptoms. It is characterized by rapid onset of acute respiratory distress syndrome; therefore, clinically, rapidly progressive BOOP can be indistinguishable from acute interstitial pneumonia. Histological features include clusters of mononuclear inflammatory cells that form granulation tissue and plug the distal airways and alveolar spaces. These plugs of granulation tissue may form polyps that migrate within the alveolar ducts or may be focally attached to the wall, (see White & Ruth-Saad (2007) Crit Care Nurse 27:53).

As used herein, the term “acute lung injury” is a clinical syndrome of acute respiratory failure and means the acute onset of diffuse bilateral pulmonary infiltrates (e.g., determined by chest radiograph), a PaO2/FiO2 less than or equal to 300 and a pulmonary artery wedge pressure of less than or equal to 18 or no clinical evidence of left atrial hypertension. “Acute respiratory distress syndrome” means the acute onset of diffuse bilateral pulmonary infiltrates (e.g., determined by chest radiograph), a PaO2/FiO2 less than or equal to 200 and a pulmonary artery wedge pressure of less than or equal to 18 or no clinical evidence of left atrial hypertension.

As used herein “fibrosis” refers to any disease or condition characterized by the formation of excess fibrous connective tissue. The formation of excess fibrous connective tissue may be in response to a reparative or reactive process. Fibrosis may be pulmonary fibrosis, liver fibrosis, myelofibrosis, skin fibrosis (e.g. nephrogenic systemic fibrosis and keloid fibrosis), mediastinal fibrosis, cardiac fibrosis, kidney fibrosis, stromal fibrosis, epidural fibrosis, epithelial fibrosis, or idiopathic fibrosis.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Patient”, “subject”, or “subject in need thereof” refers to a living organism suffering from or prone to a disease or condition that can be treated by administration of a compound or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic or therapeutic effect does not necessarily occur by administration of one dose, and may occur only after administration of a series of doses. Thus, a prophylactically effective amount or therapeutically effective amount may be administered in one or more administrations. An “activity decreasing amount,” as used herein, refers to an amount of inhibitor (e.g., antagonist) required to decrease the activity of an enzyme relative to the absence of the inhibitor (e.g., antagonist). A “function disrupting amount,” as used herein, refers to the amount of inhibitor (e.g., antagonist) required to disrupt the function of an enzyme or protein relative to the absence of the inhibitor (e.g., antagonist). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g. —NH₂, —COOH, —N-hydroxysuccinimide, or azide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONIUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful bioconjugate reactive groups used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds;
(n) azides coupled to alkynes using copper catalyzed cycloaddition click chemistry;
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease) means that the disease (e.g. cancer, fibrosis) is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propogated to other signaling pathway components.

### II. Compounds

In an aspect is provided a compound having the formula:

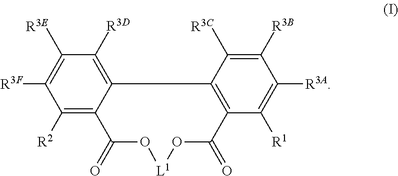

R¹ is independently hydrogen, halogen, —CX¹₃, —CHX¹₂, —CH₂X¹, —OCX¹₃, —OCH₂X¹, —OCHX¹₂, —CN, —N₃, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹ is independently hydrogen, halogen, —CX¹₃, —CHX¹₂, —CH₂X¹, —OCX¹₃, —OCH₂X¹, —OCHX¹₂. —CN, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is independently hydrogen, halogen, —CX²₃, —CHX²₂, —CH₂X², —OCX²₃, —OCH₂X², —OCHX²₂, —CN, —N₃, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is independently hydrogen, halogen, —CX²₃, —CHX²₂, —CH₂X², —OCX²₃, —OCH₂X², —OCHX²₂, —CN, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ is a substituted or unsubstituted alkylene, substituted or unsubstituted heteroalkylene, substituted or unsubstituted cycloalkylene, substituted or unsubstituted heterocycloalkyl ene, substituted or unsubstituted aryl ene, or substituted or unsubstituted heteroarylene.

R^{1A}, R^{1B}, R^{1C}, R^{1D}, R^{2A}, R^{2B}, R^{2C}, and R^{2D} are independently hydrogen, —CX₃, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3A}, R^{3B}, R^{3C}, R^{3D}, R^{3E}, and R^{3F} are independently hydrogen, —CX³₃, —CN, —N₃, —OH, —COOH, —CONH₂, —NH₂, substituted or unsubstituted alkyl, substituted or unsubstituted heteroalkyl (e.g., substituted or unsubstituted alkoxy), substituted or unsubstituted cycloalkyl, substituted or unsubstituted heterocycloalkyl, substituted or unsubstituted aryl, substituted or unsubstituted heteroaryl, —O-(substituted or unsubstituted alkyl), —O-(substituted or unsubstituted heteroalkyl), —O-(substituted or unsubstituted cycloalkyl), O(substituted or unsubstituted heterocycloalkyl), O(substituted or unsubstituted aryl), or O(substituted or unsubstituted heteroaryl). In embodiments, at least one of the R^{3A}, R^{3B}, R^{3C}, R^{3D}, R^{3E}, or R^{3F} substituents are independently —OH. In embodiments, at least two of the R^{3A}, R^{3B}, R^{3C}, R^{3D}, R^{3E}, or R^{3F} substituents are independently —OH. In embodiments, at least three of the R^{3A}, R^{3B}, R^{3C}, R^{3D}, R^{3E}, or R^{3F} substituents are independently —OH. In embodiments, at least four of the R^{3A}, R^{3B}, R^{3C}, R^{3D}, R^{3E}, or R^{3F} substituents are independently —OH. In embodiments, at least five of the R^{3A}, R^{3B}, R^{3C}, R^{3D}, R^{3E}, or R^{3F} substituents are independently —OH In embodiments, R^{3A}, R^{3B}, R^{3C}, R^{3D}, R^{3E}, and R^{3F} are independently hydrogen, —CX³₃, —CN, —OH, —COOH, —CONH₂, —NH₂, substituted or unsubstituted alkyl, substituted or unsubstituted heteroalkyl (e.g., substituted or unsubstituted alkoxy), substituted or unsubstituted cycloalkyl, substituted or unsubstituted heterocycloalkyl, substituted or unsubstituted aryl, substituted or unsubstituted heteroaryl, —O-(substituted or unsubstituted alkyl), —O-(substituted or unsubstituted heteroalkyl), —O-(substituted or unsubstituted cycloalkyl), O(substituted or unsubstituted heterocycloalkyl), O(substituted or unsubstituted aryl), or O(substituted or unsubstituted heteroaryl).

R^{1A} and R^{1B} substituents bonded to the same nitrogen atom may optionally be joined to form a substituted or unsubstituted heterocycloalkyl or substituted or unsubstituted heteroaryl. R^{2A} and R^{2B} substituents bonded to the same nitrogen atom may optionally be joined to form a substituted or unsubstituted heterocycloalkyl or substituted or unsubstituted heteroaryl.

Each X, X¹, X², and X³ is independently —F, —Cl, —Br, or —I.

In embodiments, the compound has the formula:

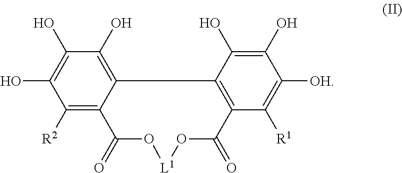

R¹, R², and L¹ are as described herein.

In embodiments, the compound has the formula:

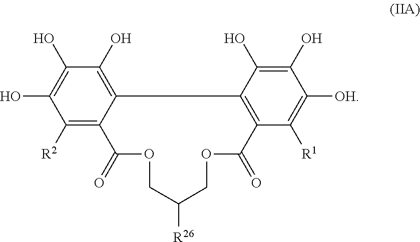

R¹, R², and R²⁶ are as described herein.

In embodiments, the compound has the formula:

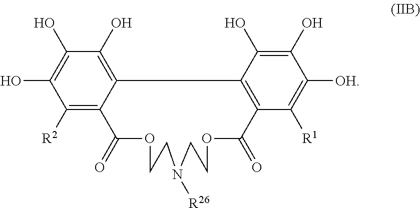

R¹, R², and R²⁶ are as described herein.

In embodiments, the compound has the formula:

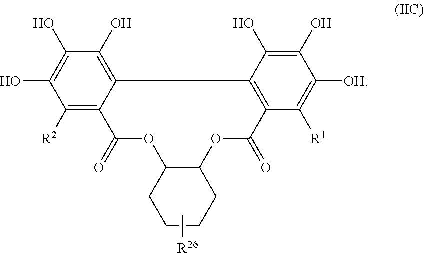

R¹, R², and R²⁶ are as described herein.

In embodiments, the compound has the formula:

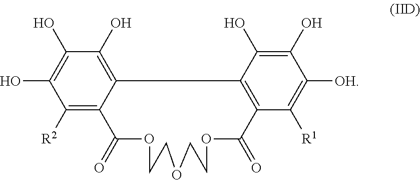

R¹ and R² are as described herein.

In embodiments, R^{3A} is —NH₂. In embodiments, R^{3A} is —OH. In embodiments, R^{3B} is —NH₂. In embodiments, R^{3B} is —OH. In embodiments, R^{3C} is —NH₂. In embodiments, R^{3C} is —OH. In embodiments, R^{3D} is —NH₂. In embodiments, R^{3D} is —OH. In embodiments, R^{3B} is —NH₂. In embodiments, R^{3E} is —OH. In embodiments, R^{3F} is —NH₂. In embodiments, R^{3F} is —OH.

In embodiments, the compound has the formula:

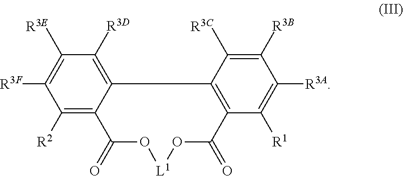

R¹, R², and L¹ are as described herein. In embodiments, R^{3A}, R^{3B}, R^{3C}, R^{3D}, and R^{3E} are —OH and R^{3F} is —NH₂. In embodiments, R^{3A}, R^{3B}, R^{3C}, R^{3D}, and R^{3F} are —OH and R^{3E} is —NH₂. In embodiments, R^{3A}, R^{3B}, R^{3C}, R^{3F}, and R^{3E} are —OH and R^{3D} is —NH₂. In embodiments, R^{3A}, R^{3B}, R^{3F}, R^{3D}, and R^{3E} are —OH and R^{3C} is —NH₂. In embodiments, R^{3A}, R^{3F}, R^{3C}, R^{3D}, and R^{3E} are —OH and R^{3B} is —NH₂. In embodiments, R^{3F}, R^{3B}, R^{3C}, R^{3D}, and R^{3E} are —OH and R^{3A} is —NH₂. In embodiments, R^{3B}, R^{3D}, R^{3E}, and R^{3F} are —OH; R^{3A} and R^{3C} are —NH₂. In embodiments, R^{3B}, R^{3C}, R^{3E}, and R^{3F} are —OH; R^{3A} and R^{3D} are —NH₂. In embodiments, R^{3B}, R^{3D}, R^{3E}, and R^{3C} are —OH; R^{3A} and R^{3F} are —NH₂. In embodiments, R^{3B}, R^{3A}, R^{3E}, and R^{3F} are —OH; R^{3D} and R^{3C} are —NH₂. In embodiments, R^{3B}, R^{3D}, R^{3E}, and R^{3A} are —OH; R^{3F} and R^{3C} are —NH₂. In embodiments, R^{3B}, R^{3A}, R^{3E}, and R^{3C} are —OH; R^{3D} and R^{3F} are —NH₂. In embodiments, R^{3A}, R^{3D}, R^{3C}, and R^{3F} are —OH; R^{3B} and R^{3E} are —NH₂. In embodiments, five of R^{3A}, R^{3B}, R^{3C}, R^{3D}, R^{3E}, and R^{3F} are —OH and one of R^{3A}, R^{3B}, R^{3C}, R^{3D}, R^{3E}, and R^{3F} is —NH₂. In embodiments, four of R^{3A}, R^{3B}, R^{3C}, R^{3D}, R^{3E}, and R^{3F} are —OH and two of R^{3A}, R^{3B}, R^{3C}, R^{3D}, R^{3E}, and R^{3F} are —NH₂. In embodiments, three of R^{3A}, R^{3B}, R^{3C}, R^{3D}, R^{3E}, and R^{3F} are —OH and three of R^{3A}, R^{3B}, R^{3C}, R^{3D}, R^{3E}, and R^{3F} are —NH₂. In embodiments, two of R^{3A}, R^{3B}, R^{3C}, R^{3D}, R^{3E}, and R^{3F} are —OH and four of R^{3A}, R^{3B}, R^{3C}, R^{3D}, R^{3E}, and R^{3F} are —NH₂. In embodiments, one of R^{3A}, R^{3B}, R^{3C}, R^{3D}, R^{3E}, and R^{3F} is —OH and five of R^{3A}, R^{3B}, R^{3C}, R^{3D}, R^{3E}, and R^{3F} are —NH₂.

In embodiments, the compound has the formula:

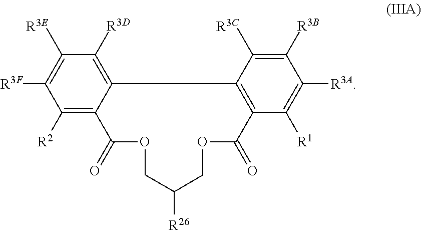

R¹, R², and R²⁶ are as described herein. In embodiments, R^{3A}, R^{3B}, R^{3C}, R^{3D}, and R^{3E} are —OH and R^{3F} is —NH₂. In embodiments, R^{3A}, R^{3B}, R^{3C}, R^{3D}, and R^{3F} are OH and R^{3E} is NH₂. In embodiments, R^{3A}, R^{3B}, R^{3C}, R^{3F}, and R^{3E} are —OH and R^{3D} is —NH₂. In embodiments, R^{3A}, R^{3B}, R^{3F}, R^{3D}, and R^{3E} are —OH and R^{3C} is —NH₂. In embodiments, R^{3A}, R^{3F}, R^{3C}, R^{3D}, and R^{3E} are —OH and R^{3B} is —NH₂. In embodiments, R^{3F}, R^{3B}, R^{3C}, R^{3D}, and R^{3E} are —OH and R^{3A} is —NH₂. In embodiments, R^{3B}, R^{3D}, R^{3E}, and R^{3F} are —OH; R^{3A} and R^{3C} are —NH₂. In embodiments, R^{3B}, R^{3C}, R^{3E}, and R^{3F} are —OH; R^{3A} and R^{3D} are —NH₂. In embodiments, R^{3B}, R^{3D}, R^{3E}, and R^{3C} are —OH; R^{3A} and R^{3F} are —NH₂. In embodiments, R^{3B}, R^{3A}, R^{3E}, and R^{3F} are —OH; R^{3D} and R^{3C} are —NH₂. In embodiments, R^{3B}, R^{3D}, R^{3E}, and R^{3A} are —OH; R^{3F} and R^{3C} are —NH₂. In embodiments, R^{3B}, R^{3A}, R^{3E}, and R^{3C} are —OH; R^{3D} and R^{3F} are —NH₂. In embodiments, R^{3A}, R^{3D}, R^{3C}, and R^{3F} are —OH; R^{3B} and R^{3E} are —NH₂. In embodiments, five of R^{3A}, R^{3B}, R^{3C}, R^{3D}, R^{3E}, and R^{3F} are —OH and one of R^{3A}, R^{3B}, R^{3C}, R^{3D}, R^{3E}, and R^{3F} is —NH₂. In embodiments, four of R^{3A}, R^{3B}, R^{3C}, R^{3D}, R^{3E}, and R^{3F} are —OH and two of R^{3A}, R^{3B}, R^{3C}, R^{3D}, R^{3E}, and R^{3F} are —NH₂. In embodiments, three of R^{3A}, R^{3B}, R^{3C}, R^{3D}, R^{3E}, and R^{3F} are —OH and three of R^{3A}, R^{3B}, R^{3C}, R^{3D}, R^{3E}, and R^{3F} are —NH₂. In embodiments, two of R^{3A}, R^{3B}, R^{3C}, R^{3D}, R^{3E}, and R^{3F} are —OH and four of R^{3A}, R^{3B}, R^{3C}, R^{3D}, R^{3E}, and R^{3F} are —NH₂. In embodiments, one of R^{3A}, R^{3B}, R^{3C}, R^{3D}, R^{3E}, and R^{3F} is —OH and five of R^{3A}, R^{3B}, R^{3C}, R^{3D}, R^{3E}, and R^{3F} are —NH₂.

In embodiments, the compound has the formula:

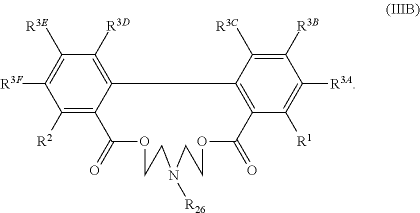

R¹, R², and R²⁶ are as described herein. In embodiments, R^{3A}, R^{3B}, R^{3C}, R^{3D}, and R^{3E} are —OH and R^{3F} is —NH₂. In embodiments, R^{3A}, R^{3B}, R^{3C}, R^{3D}, and R^{3F} are —OH and R^{3E} is —NH₂. In embodiments, R^{3A}, R^{3B}, R^{3C}, R^{3F}, and R^{3E} are —OH and R^{3D} is —NH₂. In embodiments, R^{3A}, R^{3B}, R^{3F}, R^{3D}, and R^{3E} are —OH and R^{3C} is —NH₂. In embodiments, R^{3A}, R^{3F}, R^{3C}, R^{3D}, and R^{3E} are —OH and R^{3B} is —NH₂. In embodiments, R^{3F}, R^{3B}, R^{3C}, R^{3D}, and R^{3E} are —OH and R^{3A} is —NH₂. In embodiments, R^{3B}, R^{3D}, R^{3E}, and R^{3F} are —OH; R^{3A} and R^{3C} are —NH₂. In embodiments, R^{3B}, R^{3C}, R^{3E}, and R^{3F} are —OH; R^{3A} and R^{3D} are —NH₂. In embodiments, R^{3B}, R^{3D}, R^{3E}, and R^{3C} are —OH; R^{3A} and R^{3F} are —NH₂. In embodiments, R^{3B}, R^{3A}, R^{3E}, and R^{3F} are —OH; R^{3D} and R^{3C} are —NH₂. In embodiments, R^{3B}, R^{3D}, R^{3E}, and R^{3A} are —OH; R^{3F} and R^{3C} are —NH₂. In embodiments, R^{3B}, R^{3A}, R^{3E}, and R^{3C} are —OH; R^{3D} and R^{3F} are —NH₂. In embodiments, R^{3A}, R^{3D}, R^{3C}, and R^{3F} are —OH; R^{3B} and R^{3E} are —NH₂. In embodiments, five of R^{3A}, R^{3B}, R^{3C}, R^{3D}, R^{3E}, and R^{3F} are —OH and one of R^{3A}, R^{3B}, R^{3C}, R^{3D}, R^{3E}, and R^{3F} is —NH₂. In embodiments, four of R^{3A}, R^{3B}, R^{3C}, R^{3D}, R^{3E}, and R^{3F} are —OH and two of R^{3A}, R^{3B}, R^{3C}, R^{3D}, R^{3E}, and R^{3F} are —NH₂. In embodiments, three of R^{3A}, R^{3B}, R^{3C}, R^{3D}, R^{3E}, and R^{3F} are —OH and three of R^{3A}, R^{3B}, R^{3C}, R^{3D}, R^{3E}, and R^{3F} are —NH₂. In embodiments, two of R^{3A}, R^{3B}, R^{3C}, R^{3D}, R^{3E}, and R^{3F} are —OH and four of R^{3A}, R^{3B}, R^{3C}, R^{3D}, R^{3E}, and R^{3F} are —NH₂. In embodiments, one of R^{3A}, R^{3B}, R^{3C}, R^{3D}, R^{3E}, and R^{3F} is —OH and five of R^{3A}, R^{3B}, R^{3C}, R^{3D}, R^{3E}, and R^{3F} are —NH₂.

In embodiments, the compound has the formula:

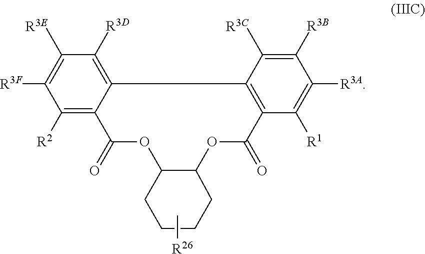

R¹, R², and R²⁶ are as described herein. In embodiments, R^{3A}, R^{3B}, R^{3C}, R^{3D}, and R^{3E} are —OH and R^{3F} is —NH₂. In embodiments, R^{3A}, R^{3B}, R^{3C}, R^{3D}, and R^{3F} are —OH and R^{3E} is —NH₂. In embodiments, R^{3A}, R^{3B}, R^{3C}, R^{3F}, and R^{3E} are —OH and R^{3D} is —NH₂. In embodiments, R^{3A}, R^{3B}, R^{3F}, R^{3D}, and R^{3E} are —OH and R^{3C} is —NH₂. In embodiments, R^{3A}, R^{3F}, R^{3C}, R^{3D}, and R^{3E} are —OH and R^{3B} is —NH₂. In embodiments, R^{3F}, R^{3B}, R^{3C}, R^{3D}, and R^{3E} are —OH and R^{3A} is —NH₂. In embodiments, R^{3B}, R^{3D}, R^{3E}, and R^{3F} are —OH; R^{3A} and R^{3C} are —NH₂. In embodiments, R^{3B}, R^{3C}, R^{3E}, and R^{3F} are —OH; R^{3A} and R^{3D} are —NH₂. In embodiments, R^{3B}, R^{3D}, R^{3E}, and R^{3C} are —OH; R^{3A} and R^{3F} are —NH₂. In embodiments, R^{3B}, R^{3A}, R^{3E}, and R^{3F} are —OH; R^{3D} and R^{3C} are —NH₂. In embodiments, R^{3B}, R^{3D}, R^{3E}, and R^{3A} are —OH; R^{3F} and R^{3C} are —NH₂. In embodiments, R^{3B}, R^{3A}, R^{3E}, and R^{3C} are —OH; R^{3D} and R^{3F} are —NH₂. In embodiments, R^{3A}, R^{3D}, R^{3C}, and R^{3F} are —OH; R^{3B} and R^{3E} are —NH₂. In embodiments, five of R^{3A}, R^{3B}, R^{3C}, R^{3D}, R^{3E}, and R^{3F} are —OH and one of R^{3A}, R^{3B}, R^{3C}, R^{3D}, R^{3E}, and R^{3F} is —NH₂. In embodiments, four of R^{3A}, R^{3B}, R^{3C}, R^{3D}, R^{3B}, and R^{3F} are —OH and two of R^{3A}, R^{3B}, R^{3C}, R^{3D}, R^{3E}, and R^{3F} are —NH₂. In embodiments, three of R^{3A}, R^{3B}, R^{3C}, R^{3D}, R^{3E}, and R^{3F} are —OH and three of R^{3A}, R^{3B}, R^{3C}, R^{3D}, R^{3E}, and R^{3F} are —NH₂. In embodiments, two of R^{3A}, R^{3B}, R^{3C}, R^{3D}, R^{3E}, and R^{3F} are —OH and four of R^{3A}, R^{3B}, R^{3C}, R^{3D}, R^{3E}, and R^{3F} are —NH₂. In embodiments, one of R^{3A}, R^{3B}, R^{3C}, R^{3D}, R^{3E}, and R^{3F} is —OH and five of R^{3A}, R^{3B}, R^{3C}, R^{3D}, R^{3E}, and R^{3F} are —NH₂.

In embodiments, the compound has the formula:

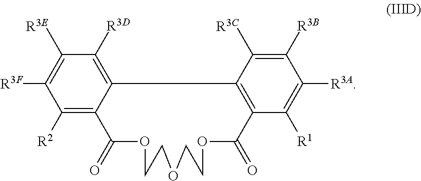

R¹ and R² are as described herein. In embodiments, R^{3A}, R^{3B}, R^{3C}, R^{3D}, and R^{3E} are —OH and R^{3F} is —NH₂. In embodiments, R^{3A}, R^{3B}, R^{3C}, R^{3D}, and R^{3F} are —OH and R^{3E} is —NH₂. In embodiments, R^{3A}, R^{3B}, R^{3C}, R^{3F}, and R^{3E} are —OH and R^{3D} is —NH₂. In embodiments, R^{3A}, R^{3B}, R^{3F}, R^{3D}, and R^{3E} are —OH and R^{3C} is —NH₂. In embodiments, R^{3A}, R^{3F}, R^{3C}, R^{3D}, and R^{3E} are —OH and R^{3B} is —NH₂. In embodiments, R^{3F}, R^{3B}, R^{3C}, R^{3D}, and R^{3E} are —OH and R^{3A} is —NH₂. In embodiments, R^{3B}, R^{3D}, R^{3E}, and R^{3F} are —OH; R^{3A} and R^{3C} are —NH₂. In embodiments, R^{3B}, R^{3C}, R^{3E}, and R^{3F} are —OH; R^{3A} and R^{3D} are —NH₂. In embodiments, R^{3B}, R^{3D}, R^{3E}, and R^{3C} are —OH; R^{3A} and R^{3F} are —NH₂. In embodiments, R^{3B}, R^{3A}, R^{3E}, and R^{3F} are —OH; R^{3D} and R^{3C} are —NH₂. In embodiments, R^{3B}, R^{3D}, R^{3E}, and R^{3A} are —OH; R^{3F} and R^{3C} are —NH₂. In embodiments, R^{3B}, R^{3A}, R^{3E}, and R^{3C} are —OH; R^{3D} and R^{3F} are —NH₂. In embodiments, R^{3A}, R^{3D}, R^{3C}, and R^{3F} are —OH; R^{3B} and R^{3E} are —NH₂. In embodiments, five of R^{3A}, R^{3B}, R^{3C}, R^{3D}, R^{3E}, and R^{3F} are —OH and one of R^{3A}, R^{3B}, R^{3C}, R^{3D}, R^{3E}, and R^{3F} is —NH₂. In embodiments, four of R^{3A}, R^{3B}, R^{3C}, R^{3D}, R^{3E}, and R^{3F} are —OH and two of R^{3A}, R^{3B}, R^{3C}, R^{3D}, R^{3E}, and R^{3F} are —NH₂. In embodiments, three of R^{3A}, R^{3B}, R^{3C}, R^{3D}, R^{3E}, and R^{3F} are —OH and three of R^{3A}, R^{3B}, R^{3C}, R^{3D}, R^{3E}, and R^{3F} are —NH₂. In embodiments, two of R^{3A}, R^{3B}, R^{3C}, R^{3D}, R^{3E}, and R^{3F} are —OH and four of R^{3A}, R^{3B}, R^{3C}, R^{3D}, R^{3E}, and R^{3F} are —NH₂. In embodiments, one of R^{3A}, R^{3B}, R^{3C}, R^{3D}, R^{3E}, and R^{3F} is —OH and five of R^{3A}, R^{3B}, R^{3C}, R^{3D}, R^{3E}, and R^{3F} are —NH₂.

In embodiments, R¹ is independently —CX¹₃. In embodiments, R¹ is independently —CHX³₂. In embodiments, R¹ is independently —CH₂X¹. In embodiments, R¹ is independently —OCX¹₃. In embodiments, R¹ is independently —OCH₂X¹. In embodiments, R¹ is independently —OCHX¹₂. In embodiments, R¹ is independently —CN. In embodiments, R¹ is independently —SR^{1D}. In embodiments, R¹ is independently —NHC(O)NR^{1A}R^{1B}. In embodiments, R¹ is independently —NR^{1A}R^{1B}. In embodiments, R¹ is independently —C(O)R^{1C}. In embodiments, R¹ is independently —C(O)OR^{1C}. In embodiments, R¹ is independently —C(O)NR^{1A}R^{1B}. In embodiments, R¹ is independently —OR^{1D}. In embodiments, R¹ is independently —NR^{1A}C(O)R^{1C}. In embodiments, R¹ is independently —NR^{1A}C(O)OR^{1C}. In embodiments, R¹ is independently —NR^{1A}OR^{1C}. In embodiments, R¹ is independently —OH. In embodiments, R¹ is independently —NH₂. In embodiments, R¹ is independently —COOH. In embodiments, R¹ is independently —CONH₂. In embodiments, R¹ is independently —SH. In embodiments, R¹ is independently hydrogen.

In embodiments, R¹ is independently hydrogen, halogen, —CX¹₃, —CN, —N₃, —OH, —NH₂, —COOH, —CONH₂, —NO₂, —SH, —NHNH₂, —ONH₂, —NHC(O)NHNH₂, —NHC(O)NH₂, —NHC(O)H, —NHC(O)OH, —NHOH, —OCX¹₃, —OCHX¹₂, R²⁰-substituted or unsubstituted C₁-C₈ alkyl (e.g., C₁-C₆, C₁-C₄, or C₁-C₂), R²⁰-substituted or unsubstituted 2 to 8 membered heteroalkyl (e.g., 2 to 6 membered, 4 to 6 membered, or 4 to 5 membered), R²⁰-substituted or unsubstituted C₃-C₈ cycloalkyl (e.g., C₃-C₆, C₄-C₆, or C₅-C₆), R²⁰-substituted or unsubstituted 3 to 6 membered heterocycloalkyl (e.g., 4 to 6 membered, 4 to 5 membered, or 5 to 6 membered), R²⁰-substituted or unsubstituted phenyl, or R²⁰-substituted or unsubstituted 5 to 6 membered heteroaryl. X¹ is —F, —Cl, —Br, or —I. In embodiments, R¹ is independently hydrogen. In embodiments, R¹ is independently methyl. In embodiments, R¹ is independently ethyl. In embodiments, R¹ is independently hydrogen, halogen, —CX¹₃, —CHX¹₂, —CH₂X¹, —OCX¹₃, —OCH₂X¹, —OCHX¹₂, —CN, —N₃, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, R²⁰-substituted or unsubstituted C₁-C₈ alkyl, R²⁰-substituted or unsubstituted 2 to 8 membered heteroalkyl. In embodiments, R¹ is independently hydrogen, oxo, halogen, —CX¹₃, —CHX¹₂, —CH₂X¹, —OCX¹₃, —OCH₂X¹, —OCHX¹₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R²⁰ is independently oxo, halogen, —CX²⁰₃, —CHX²⁰₂, —CH₂X²⁰, —OCX²⁰₃, —OCH₂X²⁰, —OCHX²⁰₂, —CN, —N₃, —SR^{20H}, —ONR^{20B}R^{20F}, —NHC(O)NR^{20E}R^{20F}, —NR^{20E}R^{20F}, —C(O)R^{20G}, —C(O)OR^{20G}, —C(O)NR^{20E}R^{20F}, —OR^{20H}, —NR^{20E}C(O)R^{20G}, —NR^{20E}C(O)OR^{20G}, —NR^{20E}OR^{20G}, R²¹-substituted or unsubstituted C₁-C₈ alkyl (e.g., C₁-C₆, C₁-C₄, or C₁-C₂), R²¹-substituted or unsubstituted 2 to 8 membered heteroalkyl (e.g., 2 to 6 membered, 4 to 6 membered, or 4 to 5 membered), R²¹-substituted or unsubstituted C₃-C₈ cycloalkyl (e.g., C₃-C₆, C₄-C₆, or C₅-C₆), R²¹-substituted or unsubstituted 3 to 6 membered heterocycloalkyl (e.g., 4 to 6 membered, 4 to 5 membered, or 5 to 6 membered), R²¹-substituted or unsubstituted C₆-C₁₀ aryl (e.g., phenyl), or R²¹-substituted or unsubstituted 5 to 10 membered heteroaryl (e.g., 5 to 9 membered or 5 to 6 membered). X²⁰ is —F, —Cl, —Br, or —I.

R^{20E}, R^{20F}, R^{20G}, and R^{20H} are independently hydrogen, oxo, halogen, —CF₃, —CHF₂, —CH₂F, —OCF₃, —OCH₂F, —OCHF₂, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl. In embodiments, R^{20E} and R^{20F} substituents bonded to the same nitrogen atom may optionally be joined to form an unsubstituted heterocycloalkyl (e.g., 3 to 8 membered, 4 to 6 membered, 4 to 5 membered, or to 6 membered) or unsubstituted heteroaryl (e.g., 5 to 10 membered, 5 to 9 membered, or 5 to 6 membered). In embodiments, R^{20E} and R^{20F} substituents bonded to the same nitrogen atom may optionally be joined to form an unsubstituted piperazinyl, unsubstituted piperidinyl, unsubstituted pyrrolidinyl, unsubstituted azetidinyl, unsubstituted morpholinyl, or unsubstituted aziridinyl.

In embodiments, R²⁰ is independently oxo, halogen, —CX²⁰₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²⁰₃, —OCHX²⁰₂, R²¹-substituted or unsubstituted C₁-C₈ alkyl (e.g., C₁-C₆, C₁-C₄, or C₁-C₂), R²¹-substituted or unsubstituted 2 to 8 membered heteroalkyl (e.g., 2 to 6 membered, 4 to 6 membered, or 4 to 5 membered), R²¹-substituted or unsubstituted C₃-C₈ cycloalkyl (e.g., C₃-C₆, C₄-C₆, or C₅-C₆), R²¹-substituted or unsubstituted 3 to 6 membered heterocycloalkyl (e.g., 4 to 6 membered, 4 to 5 membered, or to 6 membered), R²¹-substituted or unsubstituted phenyl, or R²¹-substituted or unsubstituted 5 to 6 membered heteroaryl. X²⁰ is —F, —Cl, —Br, or —I. In embodiments, R²⁰ is independently oxo, halogen, —CX^{20A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²⁰₃, —OCHX²⁰₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R²¹ is independently oxo, halogen, —CX²¹₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²¹₃, —OCHX²¹₂, R²²-substituted or unsubstituted C₁-C₈ alkyl (e.g., C₁-C₆, C₁-C₄, or C₁-C₂), R²²-substituted or unsubstituted 2 to 8 membered heteroalkyl (e.g., 2 to 6 membered, 4 to 6 membered, or 4 to 5 membered), R²²-substituted or unsubstituted C₃-C₈ cycloalkyl (e.g., C₃-C₆, C₄-C₆, or C₅-C₆), R²²-substituted or unsubstituted 3 to 6 membered heterocycloalkyl (e.g., 4 to 6 membered, 4 to 5 membered, or to 6 membered), R²²-substituted or unsubstituted phenyl, or R²²-substituted or unsubstituted to 6 membered heteroaryl. X²¹ is —F, —Cl, —Br, or —I. In embodiments, R²¹ is independently oxo, halogen, —CX²¹₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²¹₃, —OCHX²¹₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{1A} is independently hydrogen, —CX^{1A}₃, —CN, —COOH, —CONH₂, —CHX^{1A}₂, —CH₂X^{1A}, R^{20A}-substituted or unsubstituted C₁-C₈ alkyl (e.g., C₁-C₆, C₁-C₄, or C₁-C₂), R^{20A}-substituted or unsubstituted 2 to 8 membered heteroalkyl (e.g., 2 to 6 membered, 4 to 6 membered, or 4 to 5 membered), R^{20A}-substituted or unsubstituted C₃-C₈ cycloalkyl (e.g., C₃-C₆, C₄-C₆, or C₅-C₆), R^{20A}-substituted or unsubstituted 3 to 6 membered heterocycloalkyl (e.g., 4 to 6 membered, 4 to 5 membered, or 5 to 6 membered), R^{20A}-substituted or unsubstituted phenyl, or R^{20A}-substituted or unsubstituted 5 to 6 membered heteroaryl. X^{1A} is —F, —Cl, —Br, or —I. In embodiments, R^{1A} is independently hydrogen. In embodiments, R^{1A} is independently methyl. In embodiments, R^{1A} is independently ethyl. In embodiments, R^{1A} is independently hydrogen, —CX^{1A}₃, —CN, —N₃, —COOH, —CONH₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{1A} and R^{1B} substituents bonded to the same nitrogen atom may optionally be joined to form a R^{20A}-substituted or unsubstituted heterocycloalkyl or R^{20A}-substituted or unsubstituted heteroaryl. In embodiments, R^{1A} and R^{1B} substituents bonded to the same nitrogen atom may optionally be joined to form a R^{20A}-substituted or unsubstituted 3 to 6 membered heterocycloalkyl or R^{20A}-substituted or unsubstituted 5 to 6 membered heteroaryl. In embodiments, R^{1A} and R^{1B} substituents bonded to the same nitrogen atom may optionally be joined to form an unsubstituted 3 to 6 membered heterocycloalkyl or unsubstituted 5 to 6 membered heteroaryl. In embodiments, R^{1A} and R^{1B} substituents bonded to the same nitrogen atom may optionally be joined to form a R^{20A}-substituted or unsubstituted piperazinyl. In embodiments, R^{1A} and R^{1B} substituents bonded to the same nitrogen atom may optionally be joined to form a R^{20A}-substituted or unsubstituted piperidinyl. In embodiments, R^{1A} and R^{1B} substituents bonded to the same nitrogen atom may optionally be joined to form a R^{20A}-substituted or unsubstituted pyrrolidinyl. In embodiments, R^{1A} and R^{1B} substituents bonded to the same nitrogen atom may optionally be joined to form a R^{20A}-substituted or unsubstituted azetidinyl. In embodiments, R^{1A} and R^{1B} substituents bonded to the same nitrogen atom may optionally be joined to form a R^{20A}-substituted or unsubstituted morpholinyl. In embodiments, R^{1A} and R^{1B} substituents bonded to the same nitrogen atom may optionally be joined to form a R^{20A}-substituted or unsubstituted aziridinyl.

R^{20A} is independently oxo, halogen, —CX^{20A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20A}₃, —OCHX^{20A}₂, R^{21A}-substituted or unsubstituted C₁-C₈ alkyl (e.g., C₁-C₆, C₁-C₄, or C₃-C₂), R^{21A}-substituted or unsubstituted 2 to 8 membered heteroalkyl (e.g., 2 to 6 membered, 4 to 6 membered, or 4 to 5 membered), R^{21A}-substituted or unsubstituted C₃-C₈ cycloalkyl (e.g., C₃-C₆, C₄-C₆, or C₅-C₆), R^{21A}-substituted or unsubstituted 3 to 6 membered heterocycloalkyl (e.g., 4 to 6 membered, 4 to 5 membered, or 5 to 6 membered), R^{21A}-substituted or unsubstituted phenyl, or R^{21A}-substituted or unsubstituted 5 to 6 membered heteroaryl. X^{20A} is —F, —Cl, —Br, or —F In embodiments, R^{20A} is independently oxo, halogen, —CX^{20A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20A}₃, —OCHX^{20A}₂, unsubstituted C₁-C₈ alkyl (e.g., C₁-C₆, C₃-C₄, or C₃-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{21A} is independently oxo, halogen, —CX^{21A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21A}₃, —OCHX^{21A}₂, R^{22A}-substituted or unsubstituted C₃-C₈ alkyl (e.g., C₁-C₆, C₁-C₄, or C₃-C₂), R^{22A}-substituted or unsubstituted 2 to 8 membered heteroalkyl (e.g., 2 to 6 membered, 4 to 6 membered, or 4 to 5 membered), R^{22A}-substituted or unsubstituted C₃-C₈ cycloalkyl (e.g., C₃-C₆, C₄-C₆, or C₅-C₆), R^{22A}-substituted or unsubstituted 3 to 6 membered heterocycloalkyl (e.g., 4 to 6 membered, 4 to 5 membered, or 5 to 6 membered), R^{22A}-substituted or unsubstituted phenyl, or R^{22A}-substituted or unsubstituted 5 to 6 membered heteroaryl. X^{21A} is —F, —Cl, —Br, or —I. In embodiments, R^{21A} is independently oxo, halogen, —CX^{21A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21A}₃, —OCHX^{21A}₂, unsubstituted C₁-C₈ alkyl (e.g., C₁-C₆, C₃-C₄, or C₃-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{1B} is independently hydrogen, —CX^{1B}₃, —CN, —COOH, —CONH₂, —CHX^{1B}₂, —CH₂X^{1B}, R^{20B}-substituted or unsubstituted C₁-C₈ alkyl (e.g., C₁-C₈, C₁-C₄, or C₁-C₂), R^{20B}-substituted or unsubstituted 2 to 8 membered heteroalkyl (e.g., 2 to 6 membered, 4 to 6 membered, or 4 to 5 membered), R^{20B}-substituted or unsubstituted C₃-C₈ cycloalkyl (e.g., C₃-C₆, C₄-C₆, or C₅-C₆), R^{20B}-substituted or unsubstituted 3 to 6 membered heterocycloalkyl (e.g., 4 to 6 membered, 4 to 5 membered, or 5 to 6 membered), R^{20B}-substituted or unsubstituted phenyl, or R^{20B}-substituted or unsubstituted 5 to 6 membered heteroaryl. X^{1B} is —F, —Cl, —Br, or —I. In embodiments, R^{1B} is independently hydrogen. In embodiments, R^{1B} is independently methyl. In embodiments, R^{1B} is independently ethyl. In embodiments, R^{1B} is independently hydrogen, —CX^{1B}₃, —CN, —COOH, —CONH₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{1A} and R^{1B} substituents bonded to the same nitrogen atom may optionally be joined to form a R^{20B}-substituted or unsubstituted heterocycloalkyl or R^{20B}-substituted or unsubstituted heteroaryl. In embodiments, R^{1A} and R^{1B} substituents bonded to the same nitrogen atom may optionally be joined to form a R^{20B}-substituted or unsubstituted 3 to 6 membered heterocycloalkyl or R^{20B}-substituted or unsubstituted 5 to 6 membered heteroaryl. In embodiments, R^{1A} and R^{1B} substituents bonded to the same nitrogen atom may optionally be joined to form an unsubstituted 3 to 6 membered heterocycloalkyl or unsubstituted 5 to 6 membered heteroaryl. In embodiments, R^{1A} and R^{1B} substituents bonded to the same nitrogen atom may optionally be joined to form a R^{20B}-substituted or unsubstituted piperazinyl. In embodiments, R^{1A} and R^{1B} substituents bonded to the same nitrogen atom may optionally be joined to form a R^{20B}-substituted or unsubstituted piperidinyl. In embodiments, R^{1A} and R^{1B} substituents bonded to the same nitrogen atom may optionally be joined to form a R^{20B}-substituted or unsubstituted pyrrolidinyl. In embodiments, R^{1A} and R^{1B} substituents bonded to the same nitrogen atom may optionally be joined to form a R^{20B}-substituted or unsubstituted azetidinyl. In embodiments, R^{1A} and R^{1B} substituents bonded to the same nitrogen atom may optionally be joined to form a R^{20B}-substituted or unsubstituted morpholinyl. In embodiments, R^{1A} and R^{1B} substituents bonded to the same nitrogen atom may optionally be joined to form a R^{20B}-substituted or unsubstituted aziridinyl.

In embodiments, R^{20B} is independently oxo, halogen, —CX^{20B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20B}₃, —OCHX^{20B}₂, R^{21B}-substituted or unsubstituted C₁-C₈ alkyl (e.g., C₁-C₆, C₁-C₄, or C₁-C₂), R^{21B}-substituted or unsubstituted 2 to 8 membered heteroalkyl (e.g., 2 to 6 membered, 4 to 6 membered, or 4 to 5 membered), R^{21B}-substituted or unsubstituted C₃-C₈ cycloalkyl (e.g., C₃-C₆, C₄-C₆, or C₅-C₆), R^{21B}-substituted or unsubstituted 3 to 6 membered heterocycloalkyl (e.g., 4 to 6 membered, 4 to 5 membered, or 5 to 6 membered), R^{21B}-substituted or unsubstituted phenyl, or R^{21B}-substituted or unsubstituted 5 to 6 membered heteroaryl. X^{20B} is —F, —Cl, —Br, or —I. In embodiments, R^{20B} is independently oxo, halogen, —CX^{20B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20B}₃, —OCHX^{20B}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{21B} is independently oxo, halogen, —CX^{21B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21B}₃, —OCHX^{21B}₂, R^{22B}-substituted or unsubstituted C₁-C₈ alkyl (e.g., C₁-C₆, C₁-C₄, or C₁-C₂), R^{22B}-substituted or unsubstituted 2 to 8 membered heteroalkyl (e.g., 2 to 6 membered, 4 to 6 membered, or 4 to 5 membered), R^{22B}-substituted or unsubstituted C₃-C₈ cycloalkyl (e.g., C₃-C₆, C₄-C₆, or C₅-C₆), R^{22B}-substituted or unsubstituted 3 to 6 membered heterocycloalkyl (e.g., 4 to 6 membered, 4 to 5 membered, or 5 to 6 membered), R^{22B}-substituted or unsubstituted phenyl, or R^{22B}-substituted or unsubstituted 5 to 6 membered heteroaryl. X^{21B} is —F, —Cl, —Br, or —I. In embodiments, R^{21B} is independently oxo, halogen, —CX^{21B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21B}₃, —OCHX^{21B}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{1C} is independently hydrogen, —CX^{1C}₃, —CN, —N₃, —COOH, —CONH₂, —CHX^{1C}2, —CH₂X^{1C}, R^{20C}-substituted or unsubstituted C₁-C₈ alkyl (e.g., C₁-C₆, C₁-C₄, or C₁-C₂), R^{20C}-substituted or unsubstituted 2 to 8 membered heteroalkyl (e.g., 2 to 6 membered, 4 to 6 membered, or 4 to 5 membered), R^{20C}-substituted or unsubstituted C₃-C₈ cycloalkyl (e.g., C₃-C₆, C₄-C₆, or C₅-C₆), R^{20C}-substituted or unsubstituted 3 to 6 membered heterocycloalkyl (e.g., 4 to 6 membered, 4 to 5 membered, or 5 to 6 membered), R^{20C}-substituted or unsubstituted phenyl, or R^{20C}-substituted or unsubstituted 5 to 6 membered heteroaryl. X^{1C} is —F, —Cl, —Br, or —I. In embodiments, R^{1C} is independently hydrogen. In embodiments, R^{1C} is independently methyl. In embodiments, R^{1C} is independently ethyl. In embodiments, R^{1C} is independently hydrogen, —CX^{1C}₃, —CN, —COOH, —CONH₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{20C} is independently oxo, halogen, —CX^{20C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20C}₃, —OCHX^{20C}₂, R^{21C}-substituted or unsubstituted C₁-C₈ alkyl (e.g., C₁-C₆, C₄-C₄, or C₁-C₂), R^{21C}-substituted or unsubstituted 2 to 8 membered heteroalkyl (e.g., 2 to 6 membered, 4 to 6 membered, or 4 to 5 membered), R^{21C}-substituted or unsubstituted C₃-C₈ cycloalkyl (e.g., C₃-C₆, C₄-C₆, or C₅-C₆), R^{21C}-substituted or unsubstituted 3 to 6 membered heterocycloalkyl (e.g., 4 to 6 membered, 4 to 5 membered, or 5 to 6 membered), R^{21C}-substituted or unsubstituted phenyl, or R^{21C}-substituted or unsubstituted 5 to 6 membered heteroaryl. X^{20C} is —F, —Cl, —Br, or —I. In embodiments, R^{20C} is independently oxo, halogen, —CX^{20C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20C}₃, —OCHX^{20C}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{21C} is independently oxo, halogen, —CX^{21C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21C}₃, —OCHX^{21C}₂, R^{22C}-substituted or unsubstituted C₁-C₈ alkyl (e.g., C₁-C₆, C₁-C₄, or C₁-C₂), R^{22C}-substituted or unsubstituted 2 to 8 membered heteroalkyl (e.g., 2 to 6 membered, 4 to 6 membered, or 4 to 5 membered), R^{22C}-substituted or unsubstituted C₃-C₈ cycloalkyl (e.g., C₃-C₆, C₄-C₆, or C₅-C₆), R^{22C}-substituted or unsubstituted 3 to 6 membered heterocycloalkyl (e.g., 4 to 6 membered, 4 to 5 membered, or 5 to 6 membered), R^{22C}-substituted or unsubstituted phenyl, or R^{22C}-substituted or unsubstituted 5 to 6 membered heteroaryl. X^{21C} is —F, —Cl, —Br, or —I. In embodiments, R^{21C} is independently oxo, halogen, —CX^{21C}, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21C}₃, —OCHX^{21C}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{1D} is independently hydrogen, —CX^{m}₃, —CN, —COOH, —CONH₂, —CHX^{1D}₂, —CH₂X^{1D}, R^{20D}-substituted or unsubstituted C₁-C₈ alkyl (e.g., C₁-C₆. C₁-C₄, or C₁-C₂), R^{20D}-substituted or unsubstituted 2 to 8 membered heteroalkyl (e.g., 2 to 6 membered, 4 to 6 membered, or 4 to 5 membered), R^{20D}-substituted or unsubstituted C₃-C₈ cycloalkyl (e.g., C₃-C₆, C₄-C₆, or C₅-C₆), R^{20D}-substituted or unsubstituted 3 to 6 membered heterocycloalkyl (e.g., 4 to 6 membered, 4 to 5 membered, or 5 to 6 membered), R^{20D}-substituted or unsubstituted phenyl, or R^{20D}-substituted or unsubstituted 5 to 6 membered heteroaryl. X^{1D} is —F, —Cl, —Br, or —I. In embodiments, R^{m} is independently hydrogen. In embodiments, R^{1D} is independently methyl. In embodiments, R^{1D} is independently ethyl. In embodiments, R^{1D} is independently hydrogen, —CX^{1D}₃, —CN, —COOH, —CONH₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{20D} is independently oxo, halogen, —CX^{20D}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20D}₃, —OCHX^{20D}₂, R^{21D}-substituted or unsubstituted C₁-C₈ alkyl (e.g., C₁-C₆, C₁-C₄, or C₁-C₂), R^{21D}-substituted or unsubstituted 2 to 8 membered heteroalkyl (e.g., 2 to 6 membered, 4 to 6 membered, or 4 to 5 membered), R^{21D}-substituted or unsubstituted C₃-C₈ cycloalkyl (e.g., C₃-C₆, C₄-C₆, or C₅-C₆), R^{21D}-substituted or unsubstituted 3 to 6 membered heterocycloalkyl (e.g., 4 to 6 membered, 4 to 5 membered, or 5 to 6 membered), R^{21D}-substituted or unsubstituted phenyl, or R^{21D}-substituted or unsubstituted 5 to 6 membered heteroaryl. X^{20D} is —F, —Cl, —Br, or —I. In embodiments, R^{20D} is independently oxo, halogen, —CX^{20D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{20D}₃, —OCHX^{20D}₂, unsubstituted C₃-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{21D} is independently oxo, halogen, —CX^{21D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21D}₃, —OCHX^{21D}₂, R^{22D}-substituted or unsubstituted C₁-C₈ alkyl (e.g., C₁-C₆, C₁-C₄, or C₁-C₂), R^{22D}-substituted or unsubstituted 2 to 8 membered heteroalkyl (e.g., 2 to 6 membered, 4 to 6 membered, or 4 to 5 membered), R^{22D}-substituted or unsubstituted C₃-C₈ cycloalkyl (e.g., C₃-C₆, C₄-C₆, or C₅-C₆), R^{22D}-substituted or unsubstituted 3 to 6 membered heterocycloalkyl (e.g., 4 to 6 membered, 4 to 5 membered, or 5 to 6 membered), R^{22D}-substituted or unsubstituted phenyl, or R^{22D}-substituted or unsubstituted 5 to 6 membered heteroaryl. X^{21D} is —F, —Cl, —Br, or —I. In embodiments, R^{21D} is independently oxo, halogen, —CX^{21D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{21D}₃, —OCHX^{21D}₂, unsubstituted C₁-C₈ alkyl (e.g., C₁-C₆, C₁-C₄, or C₁—C>), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R²², R^{22A}, R^{22B}, R^{22C}, and R^{22D} are independently oxo, halogen, —CF₃, —CHF₂, —CH₂F, —OCF₃, —OCH₂F, —OCHF₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12, 5 to 10 membered, 5 to 9 membered, or 5 to 6 membered).

In embodiments, R²², R^{22A}, R^{22B}, R^{22C}, and R^{22D} are independently oxo, halogen, —CF₃, —CHF₂, —CH₂F, —OCF₃, —OCH₂F, —OCHF₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R¹ is independently halogen. In embodiments, R¹ is independently hydrogen.

In embodiments, R² is independently halogen. In embodiments, R² is independently —CX²₃. In embodiments, R² is independently —CHX²₂. In embodiments, R² is independently —CH₂X². In embodiments, R² is independently —OCX²₃. In embodiments, R² is independently —OCH₂X². In embodiments, R² is independently —OCHX²₂. In embodiments, R² is independently —CN. In embodiments, R² is independently —SR^{2D}. In embodiments, R² is independently —NHC(O)NR^{2A}R^{2B}. In embodiments, R² is independently —NR^{2A}R^{2B}. In embodiments, R² is independently —C(O)R^{2C}. In embodiments, R² is independently —C(O)OR^{2C}. In embodiments, R² is independently —C(O)NR^{2A}R^{2B}. In embodiments, R² is independently —OR^{2D}. In embodiments, R² is independently —NR^{2A}C(O)R^{2C}. In embodiments, R² is independently —NR^{2A}C(O)OR^{2C}. In embodiments, R² is independently —NR^{2A}OR^{2C}. In embodiments, R² is independently —OH. In embodiments, R² is independently —NH₂. In embodiments, R² is independently —COOH. In embodiments, R² is independently —CONH₂. In embodiments, R² is independently —SH. In embodiments, R² is independently hydrogen.

In embodiments, R² is independently hydrogen, halogen, —CX²₃, —CHX²₂, —CH₂X², —OCX²₃, —OCH₂X², —OCHX²₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, R²³-substituted or unsubstituted C₁-C₈ alkyl (e.g., C₁-C₆, C₁-C₄, or C₁-C₂), R²³-substituted or unsubstituted 2 to 8 membered heteroalkyl (e.g., 2 to 6 membered, 4 to 6 membered, or 4 to 5 membered), R²³-substituted or unsubstituted C₃-C₈ cycloalkyl (e.g., C₃-C₆, C₄-C₆, or C₅-C₆), R²³-substituted or unsubstituted 3 to 6 membered heterocycloalkyl (e.g., 4 to 6 membered, 4 to 5 membered, or 5 to 6 membered), R²³-substituted or unsubstituted phenyl, or R²³-substituted or unsubstituted 5 to 6 membered heteroaryl. X² is —F, —Cl, —Br, or —I. In embodiments, R² is independently hydrogen. In embodiments, R² is independently methyl. In embodiments, R² is independently ethyl. In embodiments, R² is independently hydrogen, halogen, —CX²₃, —CHX²₂, —CH₂X², —OCX²₃, —OCH₂X², —OCHX²₂, —CN, —N₃, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, R²³-substituted or unsubstituted C₁-C₈ alkyl, or R²³-substituted or unsubstituted 2 to 8 membered heteroalkyl.

R²³ is independently oxo, halogen, —CX²³₃, —CHX²³₂, —CH₂X²³, —OCX²³₃, —OCH₂X²³, —OCHX²³₂, —CN, —N₃, —SR^{23H}, —ONR^{23B}R^{23F}, —NHC(O)NR^{23E}R^{23F}, —NR^{23E}R^{23F}, —C(O)R^{23G}, —C(O)OR^{23G}, —C(O)NR^{23E}R^{23F}, —OR^{23H}, —NR^{23E}C(O)R^{23G}, —NR^{23E}C(O)OR^{23G}, —NR^{23E}OR^{23G}, R²⁴-substituted or unsubstituted C₁-C₈ alkyl (e.g., C₁-C₆, C₁-C₄, or C₁-C₂), R²⁴-substituted or unsubstituted 2 to 8 membered heteroalkyl (e.g., 2 to 6 membered, 4 to 6 membered, or 4 to 5 membered), R²⁴-substituted or unsubstituted C₃-C₈ cycloalkyl (e.g., C₃-C₆, C₄-C₆, or C₅-C₆), R²⁴-substituted or unsubstituted 3 to 6 membered heterocycloalkyl (e.g., 4 to 6 membered, 4 to 5 membered, or 5 to 6 membered), R²⁴-substituted or unsubstituted C₆-C₁₀ aryl (e.g., phenyl), or R²⁴-substituted or unsubstituted 5 to 10 membered heteroaryl (e.g., to 9 membered or 5 to 6 membered). X²³ is —F, —Cl, —Br, or —I. In embodiments, R²³ is independently oxo, halogen, —CX²³₃, —CHX²³₂, —CH₂X²³, —OCX²³₃, —OCH₂X²³, —OCHX²³₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₃-C₄, or C₃-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{23E}, R^{23F}, R^{23G}, and R^{23H} are independently hydrogen, oxo, halogen, —CF₃, —CHF₂, —CH₂F, —OCF₃, —OCH₂F, —OCHF₂, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₃-C₈ alkyl (e.g., C₁-C₆, C₃-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl. In embodiments, R^{23B} and R^{23F} substituents bonded to the same nitrogen atom may optionally be joined to form an unsubstituted heterocycloalkyl (e.g., 3 to 8 membered, 4 to 6 membered, 4 to 5 membered, or to 6 membered) or unsubstituted heteroaryl (e.g., 5 to 10 membered, 5 to 9 membered, or 5 to 6 membered). In embodiments, R^{23E} and R^{23F} substituents bonded to the same nitrogen atom may optionally be joined to form an unsubstituted piperazinyl, unsubstituted piperidinyl, unsubstituted pyrrolidinyl, unsubstituted azetidinyl, unsubstituted morpholinyl, or unsubstituted aziridinyl.

In embodiments, R²³ is independently oxo, halogen, —CX²³₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²³₃, —OCHX²³₂, R²⁴-substituted or unsubstituted C₁-C₈ alkyl (e.g., C₁-C₆, C₁-C₄, or C₁-C₂), R²⁴-substituted or unsubstituted 2 to 8 membered heteroalkyl (e.g., 2 to 6 membered, 4 to 6 membered, or 4 to 5 membered), R²⁴-substituted or unsubstituted C₃-C₈ cycloalkyl (e.g., C₃-C₆, C₄-C₆, or C₅-C₆), R²⁴-substituted or unsubstituted 3 to 6 membered heterocycloalkyl (e.g., 4 to 6 membered, 4 to 5 membered, or to 6 membered), R²⁴-substituted or unsubstituted phenyl, or R²⁴-substituted or unsubstituted to 6 membered heteroaryl. X²³ is —F, —Cl, —Br, or —I. In embodiments, R²³ is independently oxo, halogen, —CX²³₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²³₃, —OCHX²³₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R²⁴ is independently oxo, halogen, —CX²⁴₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²⁴₃, —OCHX²⁴₂, R²⁵-substituted or unsubstituted C₁-C₈ alkyl (e.g., C₁-C₆. C₁-C₄, or C₁-C₂), R²⁵-substituted or unsubstituted 2 to 8 membered heteroalkyl (e.g., 2 to 6 membered, 4 to 6 membered, or 4 to 5 membered), R²⁵-substituted or unsubstituted C₃-C₈ cycloalkyl (e.g., C₃-C₆, C₄-C₆, or C₅-C₆), R²⁵-substituted or unsubstituted 3 to 6 membered heterocycloalkyl (e.g., 4 to 6 membered, 4 to 5 membered, or 5 to 6 membered), R²⁵-substituted or unsubstituted phenyl, or R²⁵-substituted or unsubstituted 5 to 6 membered heteroaryl. X²⁴ is —F, —Cl, —Br, or —F In embodiments, R²⁴ is independently oxo, halogen, —CX²⁴₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX²⁴₃, —OCHX²⁴₂, unsubstituted C₁-C₈ alkyl (e.g., C₁-C₆, C₁-C₄, or C₄-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or to 6 membered), unsubstituted phenyl, or unsubstituted 5 to 6 membered heteroaryl.

In embodiments, R^{2A} is independently hydrogen, —CX^{2A}₃, —CN, —COOH, —CONH₂, —CHX^{2A}₂, —CH₂X^{2A}, R^{23A}-substituted or unsubstituted C₁-C₈ alkyl (e.g., C₁-C₆, C₁-C₄, or C₁-C₂), R^{23A}-substituted or unsubstituted 2 to 8 membered heteroalkyl (e.g., 2 to 6 membered, 4 to 6 membered, or 4 to 5 membered), R^{23A}-substituted or unsubstituted C₃-C₈ cycloalkyl (e.g., C₃-C₆, C₄-C₆, or C₅-C₆), R^{23A}-substituted or unsubstituted 3 to 6 membered heterocycloalkyl (e.g., 4 to 6 membered, 4 to 5 membered, or 5 to 6 membered), R^{23A}-substituted or unsubstituted phenyl, or R^{23A}-substituted or unsubstituted 5 to 6 membered heteroaryl. X^{2A} is —F, —Cl, —Br, or —I. In embodiments, R^{2A} is independently hydrogen. In embodiments, R^{2A} is independently methyl. In embodiments, R^{2A} is independently ethyl. In embodiments, R^{2A} is independently hydrogen, —CX^{2A}₃, —CN, —COOH, —CONH₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{2A} and R^{2B} substituents bonded to the same nitrogen atom may optionally be joined to form a R^{23A}-substituted or unsubstituted 3 to 6 membered heterocycloalkyl (e.g., 4 to 6 membered, 4 to 5 membered, or 5 to 6 membered) or R^{23A}-substituted or unsubstituted 5 to 6 membered heteroaryl. In embodiments, R^{2A} and R^{2B} substituents bonded to the same nitrogen atom may optionally be joined to form an unsubstituted 3 to 6 membered heterocycloalkyl or unsubstituted 5 to 6 membered heteroaryl. In embodiments, R^{2A} and R^{2B} substituents bonded to the same nitrogen atom may optionally be joined to form a R^{23A}-substituted or unsubstituted piperazinyl. In embodiments, R^{2A} and R^{2B} substituents bonded to the same nitrogen atom may optionally be joined to form a R^{23A}-substituted or unsubstituted piperidinyl. In embodiments, R^{2A} and R^{2B} substituents bonded to the same nitrogen atom may optionally be joined to form a R^{23A}-substituted or unsubstituted pyrrolidinyl. In embodiments, R^{2A} and R^{2B} substituents bonded to the same nitrogen atom may optionally be joined to form a R^{23A}-substituted or unsubstituted azetidinyl. In embodiments, R^{2A} and R^{2B} substituents bonded to the same nitrogen atom may optionally be joined to form a R^{23A}-substituted or unsubstituted morpholinyl. In embodiments, R^{2A} and R^{2B} substituents bonded to the same nitrogen atom may optionally be joined to form a R^{23A}-substituted or unsubstituted aziridinyl.

R^{23A} is independently oxo, halogen, —CX^{23A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23A}₃, —OCHX^{23A}₂, R^{24A}-substituted or unsubstituted C₁-C₈ alkyl (e.g., C₁-C₆, C₁-C₄, or C₁-C₂), R^{24A}-substituted or unsubstituted 2 to 8 membered heteroalkyl (e.g., 2 to 6 membered, 4 to 6 membered, or 4 to 5 membered), R^{24A}-substituted or unsubstituted C₃-C₈ cycloalkyl (e.g., C₃-C₆, C₄-C₆, or C₅-C₆), R^{24A}-substituted or unsubstituted 3 to 6 membered heterocycloalkyl (e.g., 4 to 6 membered, 4 to 5 membered, or 5 to 6 membered), R^{24A}-substituted or unsubstituted phenyl, or R^{24A}-substituted or unsubstituted 5 to 6 membered heteroaryl. X^{23A} is —F, —Cl, —Br, or —I.

In embodiments, R^{23A} is independently oxo, halogen, —CX^{23A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23A}₃, —OCHX^{23A}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{24A} is independently oxo, halogen, —CX^{24A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24A}₃, —OCHX^{24A}₂, R^{25A}-substituted or unsubstituted C₁-C₈ alkyl (e.g., C₁-C₆, C₁-C₄, or C₁-C₂), R^{25A}-substituted or unsubstituted 2 to 8 membered heteroalkyl (e.g., 2 to 6 membered, 4 to 6 membered, or 4 to 5 membered), R^{25A}-substituted or unsubstituted C₃-C₈ cycloalkyl (e.g., C₃-C₆, C₄-C₆, or C₅-C₆), R^{25A}-substituted or unsubstituted 3 to 6 membered heterocycloalkyl (e.g., 4 to 6 membered, 4 to 5 membered, or 5 to 6 membered), R^{25A}-substituted or unsubstituted phenyl, or R^{25A}-substituted or unsubstituted 5 to 6 membered heteroaryl. X^{24A} is —F, —Cl, —Br, or —I. In embodiments, R^{24A} is independently oxo, halogen, —CX^{24A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24A}₃, —OCHX^{24A}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{2B} is independently hydrogen, —CX^{2B}₃, —CN, —COOH, —CONH₂, —CHX^{2B}₂, —CH₂X^{2B}, R^{23B}-substituted or unsubstituted C₁-C₈ alkyl (e.g., C₁-C₆, C₁-C₄, or C₁-C₂), R^{23B}-substituted or unsubstituted 2 to 8 membered heteroalkyl (e.g., 2 to 6 membered, 4 to 6 membered, or 4 to 5 membered), R^{23B}-substituted or unsubstituted C₃-C₈ cycloalkyl (e.g., C₃-C₆, C₄-C₆, or C₅-C₆), R^{23B}-substituted or unsubstituted 3 to 6 membered heterocycloalkyl (e.g., 4 to 6 membered, 4 to 5 membered, 23B 23B or 5 to 6 membered), R^{23B}-substituted or unsubstituted phenyl, or R^{23B}-substituted or unsubstituted 5 to 6 membered heteroaryl. X^{2B} is —F, —Cl, —Br, or —I. In embodiments, R^{2B} is independently hydrogen. In embodiments, R^{2B} is independently methyl. In embodiments, R^{2B} is independently ethyl. In embodiments, R^{2B} is independently hydrogen, —CX^{2B}₃, —CN, —COOH, —CONH₂, unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{2A} and R^{2B} substituents bonded to the same nitrogen atom may optionally be joined to form a R^{23B}-substituted or unsubstituted 3 to 6 membered heterocycloalkyl (e.g., 4 to 6 membered, 4 to 5 membered, or 5 to 6 membered) or R^{23B}-substituted or unsubstituted 5 to 6 membered heteroaryl. In embodiments, R^{2A} and R^{2B} substituents bonded to the same nitrogen atom may optionally be joined to form an unsubstituted 3 to 6 membered heterocycloalkyl or unsubstituted 5 to 6 membered heteroaryl. In embodiments, R^{2A} and R^{2B} substituents bonded to the same nitrogen atom may optionally be joined to form a R^{23B}-substituted or unsubstituted piperazinyl. In embodiments, R^{2A} and R^{2B} substituents bonded to the same nitrogen atom may optionally be joined to form a R^{23B}-substituted or unsubstituted piperidinyl. In embodiments, R^{2A} and R^{2B} substituents bonded to the same nitrogen atom may optionally be joined to form a R^{23B}-substituted or unsubstituted pyrrolidinyl. In embodiments, R^{2A} and R^{2B} substituents bonded to the same nitrogen atom may optionally be joined to form a R^{23B}-substituted or unsubstituted azetidinyl. In embodiments, R^{2A} and R^{2B} substituents bonded to the same nitrogen atom may optionally be joined to form a R^{23B}-substituted or unsubstituted morpholinyl. In embodiments, R^{2A} and R^{2B} substituents bonded to the same nitrogen atom may optionally be joined to form a R^{23B}-substituted or unsubstituted aziridinyl.

R^{23B} is independently oxo, halogen, —CX^{23B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23B}₃, —OCHX^{23B}₂, R^{24B}-substituted or unsubstituted C₁-C₈ alkyl (e.g., C₁-C₆, C₁-C₄, or C₁-C₂), R^{24B}-substituted or unsubstituted 2 to 8 membered heteroalkyl (e.g., 2 to 6 membered, 4 to 6 membered, or 4 to 5 membered), R^{24B}-substituted or unsubstituted C₃-C₈ cycloalkyl (e.g., C₃-C₆, C₄-C₆, or C₅-C₆), R^{24B}-substituted or unsubstituted 3 to 6 membered heterocycloalkyl (e.g., 4 to 6 membered, 4 to 5 membered, or 5 to 6 membered), R^{24B}-substituted or unsubstituted phenyl, or R^{24B}-substituted or unsubstituted 5 to 6 membered heteroaryl. X^{23B} 23B 23B is —F, —Cl, —Br, or —I. In embodiments, R^{23B} is independently oxo, halogen, —CX^{23B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23B}₃, —OCHX^{23B}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{24B} is independently oxo, halogen, —CX^{24B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24B}₃, —OCHX^{24B}₂, R^{25B}-substituted or unsubstituted C₁-C₈ alkyl (e.g., C₁-C₆, C₁-C₄, or C₁-C₂), R^{25B}-substituted or unsubstituted 2 to 8 membered heteroalkyl (e.g., 2 to 6 membered, 4 to 6 membered, or 4 to 5 membered), R^{25B}-substituted or unsubstituted C₃-C₈ cycloalkyl (e.g., C₃-C₆, C₄-C₆, or C₅-C₆), R^{25B}-substituted or unsubstituted 3 to 6 membered heterocycloalkyl (e.g., 4 to 6 membered, 4 to 5 membered, or 5 to 6 membered), R^{25B}-substituted or unsubstituted phenyl, or R^{25B}-substituted or unsubstituted 5 to 6 membered heteroaryl. X^{24B} is —F, —Cl, —Br, or —I. In embodiments, R^{25B} is independently oxo, halogen, —CX₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24B}₃, —OCHX^{24B}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{2C} is independently hydrogen, —CX^{2C}₃, —CN, —N₃, —COOH, —CONH₂, —CHX^{2C}₂, —CH₂X^{2C}, R^{23C}-substituted or unsubstituted C₁-C₈ alkyl (e.g., C₁-C₆, C₁-C₄, or C₁-C₂), R^{23C}-substituted or unsubstituted 2 to 8 membered heteroalkyl (e.g., 2 to 6 membered, 4 to 6 membered, or 4 to 5 membered), R^{23C}-substituted or unsubstituted C₃-C₈ cycloalkyl (e.g., C₃-C₆, C₄-C₆, or C₅-C₆), R^{23C}-substituted or unsubstituted 3 to 6 membered heterocycloalkyl (e.g., 4 to 6 membered, 4 to 5 membered, or 5 to 6 membered), R^{23C}-substituted or unsubstituted phenyl, or R^{23C}-substituted or unsubstituted 5 to 6 membered heteroaryl. X^{2C} is —F, —Cl, —Br, or —I. In embodiments, R^{2C} is independently hydrogen. In embodiments, R^{2C} is independently methyl. In embodiments, R^{2C} is independently ethyl. In embodiments, R^{2C} is independently hydrogen, —CX^{2C} 3, —CN, —COOH, —CONH₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{23C} is independently oxo, halogen, —CX^{23C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23C}₃, —OCHX^{23C}₂, R^{24C}-substituted or unsubstituted C₁-C₈ alkyl (e.g., C₁-C₆, C₁-C₄, or C₃-C₂), R^{24C}-substituted or unsubstituted 2 to 8 membered heteroalkyl (e.g., 2 to 6 membered, 4 to 6 membered, or 4 to 5 membered), R^{24C}-substituted or unsubstituted C₃-C₈ cycloalkyl (e.g., C₃-C₆, C₄-C₆, or C₅-C₆), R^{24C}-substituted or unsubstituted 3 to 6 membered heterocycloalkyl (e.g., 4 to 6 membered, 4 to 5 membered, or 5 to 6 membered), R^{24C}-substituted or unsubstituted phenyl, or R^{24C}-substituted or unsubstituted 5 to 6 membered heteroaryl. X^{23C} is —F, —Cl, —Br, or —I. In embodiments, R^{24C} is independently oxo, halogen, —CX^{23C}, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23C}₃, —OCHX^{23C}₂, unsubstituted C₁-C₈ alkyl (e.g., C₁-C₆, C₁-C₄, or C₃-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{24C} is independently oxo, halogen, —CX^{24C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24C}₃, —OCHX^{24C}₂, R^{25C}-substituted or unsubstituted C₁-C₈ alkyl (e.g., C₁-C₆, C₁-C₄, or C₁-C₂), R^{25C}-substituted or unsubstituted 2 to 8 membered heteroalkyl (e.g., 2 to 6 membered, 4 to 6 membered, or 4 to 5 membered), R^{25C}-substituted or unsubstituted C₃-C₈ cycloalkyl (e.g., C₃-C₆, C₄-C₆, or C₅-C₆), R^{25C}-substituted or unsubstituted 3 to 6 membered heterocycloalkyl (e.g., 4 to 6 membered, 4 to 5 membered, or 5 to 6 membered), R^{25C}-substituted or unsubstituted phenyl, or R^{25C}-substituted or unsubstituted 5 to 6 membered heteroaryl. X^{24C} is —F, —Cl, —Br, or —I. In embodiments, R^{24C} is independently oxo, halogen, —CX^{24C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24C}₃, —OCHX^{24C}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{2D} is independently hydrogen, —CX^{2D}₃, —CN, —COOH, —CONH₂, —CHX^{2D}₂, —CH₂X^{2D}, R^{23D}-substituted or unsubstituted C₁-C₈ alkyl (e.g., C₁-C₆, C₁-C₄, or C₁-C₂), R^{23D}-substituted or unsubstituted 2 to 8 membered heteroalkyl (e.g., 2 to 6 membered, 4 to 6 membered, or 4 to 5 membered), R^{23D}-substituted or unsubstituted C₃-C₈ cycloalkyl (e.g., C₃-C₆, C₄-C₆, or C₅-C₆), R^{23D}-substituted or unsubstituted 3 to 6 membered heterocycloalkyl (e.g., 4 to 6 membered, 4 to 5 membered, or 5 to 6 membered), R^{23D}-substituted or unsubstituted phenyl, or R^{23D}-substituted or unsubstituted 5 to 6 membered heteroaryl. X^{2D} is —F, —Cl, —Br, or —I. In embodiments, R^{2D} is independently hydrogen. In embodiments, R^{2D} is independently methyl. In embodiments, R is independently ethyl. In embodiments, R^{2D} is independently hydrogen, —CX^{2D}₃, —CN, —COOH, —CONH₂, unsubstituted C₁-C₈ alkyl (e.g., C₁-C₆, C₃-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{23D} is independently oxo, halogen, —CX^{23D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23D}₃, —OCHX^{23D}₂, R^{24D}-substituted or unsubstituted C₁-C₈ alkyl (e.g., C₁-C₆, C₁-C₄, or C₁-C₂), R^{24D}-substituted or unsubstituted 2 to 8 membered heteroalkyl (e.g., 2 to 6 membered, 4 to 6 membered, or 4 to 5 membered), R^{24D}-substituted or unsubstituted C₃-C₈ cycloalkyl (e.g., C₃-C₆, C₄-C₆, or C₅-C₆), R^{24D}-substituted or unsubstituted 3 to 6 membered heterocycloalkyl (e.g., 4 to 6 membered, 4 to 5 membered, or 5 to 6 membered), R^{24D}-substituted or unsubstituted phenyl, or R^{24D}-substituted or unsubstituted 5 to 6 membered heteroaryl. X^{23D} is F, —Cl, —Br, or —I. In embodiments, R^{23D} is independently oxo, halogen, —CX^{23D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{23D}₃, —OCHX^{23D}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{24D} is independently oxo, halogen, —CX^{24D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24D}₃, —OCHX^{24D}₂, R^{25D}-substituted or unsubstituted C₁-C₈ alkyl (e.g., C₁-C₆, C₁-C₄, or C₁-C₂), R^{25D}-substituted or unsubstituted 2 to 8 membered heteroalkyl (e.g., 2 to 6 membered, 4 to 6 membered, or 4 to 5 membered), R^{25D}-substituted or unsubstituted C₃-C₈ cycloalkyl (e.g., C₃-C₆, C₄-C₆, or C₅-C₆), R^{25D}-substituted or unsubstituted 3 to 6 membered heterocycloalkyl (e.g., 4 to 6 membered, 4 to 5 membered, or 5 to 6 membered), R^{25D}-substituted or unsubstituted phenyl, or R^{25D}-substituted or unsubstituted 5 to 6 membered heteroaryl. X^{24D} is —F, —Cl, —Br, or —I. In embodiments, R^{24D} is independently oxo, halogen, —CX^{24D}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{24D}₃, —OCHX^{24D}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R²⁵, R^{25A}, R^{25B}, R^{25C}, and R^{25D} are independently oxo, halogen, —CF₃, —CHF₂, —CH₂F, —OCF₃, —OCH₂F, —OCHF₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R² is independently halogen. In embodiments, R² is independently hydrogen.

In embodiments, L¹ is an R²⁶-substituted C₂-C₉ alkylene (e.g., C₂-C₈, C₄-C₆, or C₄-C₅), R²⁶-substituted or unsubstituted 2 to 9 membered heteroalkylene (e.g., 2 to 8 membered, 4 to 6 membered, or 4 to 5 membered), or R²⁶-substituted or unsubstituted C₃-C₈ cycloalkylene (e.g., C₃-C₆, C₄-C₆, or C₅-C₆). In embodiments, L¹ is an R²⁶-substituted C₂-C₉ alkylene (e.g., C₂-C₈, C₄-C₆, or C₄-C₅). In embodiments, L¹ is an R²⁶-substituted or unsubstituted 2 to 9 membered heteroalkylene (e.g., 2 to 8 membered, 4 to 6 membered, or 4 to 5 membered). In embodiments, L¹ is an R²⁶-substituted or unsubstituted C₃-C₈ cycloalkylene (e.g., C₃-C₆, C₄-C₆, or C₅-C₆). In embodiments, L¹ is an R²⁶-substituted C₂-C₉ alkylene, R²⁶-substituted or unsubstituted 2 to 9 membered heteroalkylene, or R²⁶-substituted or unsubstituted C₃-C₈ cycloalkylene.

R²⁶ is N₃, —COOR^{26C}, —CONR^{26A}R^{26B}, —NR^{26D}C(O)NR^{26A}R^{26B}, —NR^{26A}C(O)OR^{26C}, —C(O)R^{26C}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R²⁶ is R²⁷-substituted or unsubstituted alkyl, R²⁷-substituted or unsubstituted heteroalkyl, R²⁷-substituted or unsubstituted cycloalkyl, R²⁷-substituted or unsubstituted heterocycloalkyl, R²⁷-substituted or unsubstituted aryl, R²⁷-substituted or unsubstituted heteroaryl.

R²⁷ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁸-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R²⁸-substituted or unsubstituted 2 to 8 membered heteroalkyl (e.g., 2 to 6 membered, 4 to 6 membered, or 4 to 5 membered), R²⁸-substituted or unsubstituted C₃-C₈ cycloalkyl (e.g., C₃-C₆, C₄-C₆, or C₅-C₆), R²⁸-substituted or unsubstituted 3 to 6 membered heterocycloalkyl (e.g., 4 to 6 membered, 4 to 5 membered, or 5 to 6 membered), R²⁸-substituted or unsubstituted C₆-C₁₀ aryl (e.g., phenyl), or R²⁸-substituted or unsubstituted 5 to 10 membered heteroaryl (e.g., 5 to 9 membered or 5 to 6 membered).

In embodiments, R²⁷ is R²⁸-substituted or unsubstituted alkyl (e.g., C₁-C₈ alkyl, C₁-C₆ alkyl, or C₁-C₄ alkyl). In embodiments, R²⁷ is R²⁸-substituted alkyl (e.g., C₁-C₈ alkyl, C₁-C₆ alkyl, or C₁-C₄ alkyl). In embodiments, R²⁷ is an unsubstituted alkyl (e.g., C₁-C₈ alkyl, C₁-C₆ alkyl, or C₁-C₄ alkyl).

In embodiments, R²⁷ is R²⁸-substituted or unsubstituted heteroalkyl (e.g., 2 to 8 membered heteroalkyl, 2 to 6 membered heteroalkyl, or 2 to 4 membered heteroalkyl). In embodiments, R²⁷ is R²⁸-substituted heteroalkyl (e.g., 2 to 8 membered heteroalkyl, 2 to 6 membered heteroalkyl, or 2 to 4 membered heteroalkyl). In embodiments, R²⁷ is an unsubstituted heteroalkyl (e.g., 2 to 8 membered heteroalkyl, 2 to 6 membered heteroalkyl, or 2 to 4 membered heteroalkyl).

In embodiments, R²⁷ is R²⁸-substituted or unsubstituted cycloalkyl (e.g., C₃-C₈ cycloalkyl, C₃-C₆ cycloalkyl, or C₅-C₆ cycloalkyl). In embodiments, R²⁷ is R²⁸-substituted cycloalkyl (e.g., C₃-C₈ cycloalkyl, C₃-C₆ cycloalkyl, or C₅-C₆ cycloalkyl). In embodiments, R²⁷ is an unsubstituted cycloalkyl (e.g., C₃-C₈ cycloalkyl, C₃-C₆ cycloalkyl, or C₅-C₆ cycloalkyl).

In embodiments, R²⁷ is R²⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁷ is R²⁸-substituted heterocycloalkyl (e.g., 3 to 8 membered heterocycloalkyl, 3 to 6 membered heterocycloalkyl, or 5 to 6 membered heterocycloalkyl). In embodiments, R²⁷ is an unsubstituted heterocycloalkyl (e.g., 3 to 8 membered heterocycloalkyl, 3 to 6 membered heterocycloalkyl, or 5 to 6 membered heterocycloalkyl).

In embodiments, R²⁷ is R²⁸-substituted or unsubstituted aryl (e.g., C₆-C₁₀ aryl, C₁₀ aryl, or phenyl). In embodiments, R²⁷ is R²⁸-substituted aryl (e.g., C₆-C₁₀ aryl, C₁₀ aryl, or phenyl). In embodiments, R²⁷ is an unsubstituted aryl (e.g., C₆-C₁₀ aryl, C₁₀ aryl, or phenyl).

In embodiments, R²⁷ is R²⁸-substituted or unsubstituted heteroaryl (e.g., 5 to 10 membered heteroaryl, 5 to 9 membered heteroaryl, or 5 to 6 membered heteroaryl). In embodiments, R²⁷ is R²⁸-substituted heteroaryl (e.g., 5 to 10 membered heteroaryl, 5 to 9 membered heteroaryl, or 5 to 6 membered heteroaryl). In embodiments, R²⁷ is an unsubstituted heteroaryl (e.g., 5 to 10 membered heteroaryl, 5 to 9 membered heteroaryl, or 5 to 6 membered heteroaryl).

R²⁸ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁹-substituted or unsubstituted C₁-C₈ alkyl (e.g., C₁-C₆, C₃-C₄, C₃-C₂, C₂-C₆ alkenyl, C₂-C₄ alkenyl, C₂-C₆ alkynyl, or C₂-C₄ alkynyl), R²⁹-substituted or unsubstituted 2 to 8 membered heteroalkyl (e.g., 2 to 6 membered, 4 to 6 membered, or 4 to 5 membered), R²⁹-substituted or unsubstituted C₃-C₈ cycloalkyl (e.g., C₃-C₆, C₄-C₆, or C₅-C₆), R²⁹-substituted or unsubstituted 3 to 6 membered heterocycloalkyl (e.g., 4 to 6 membered, 4 to 5 membered, or 5 to 6 membered), R²⁹-substituted or unsubstituted C₆-C₁₀ aryl (e.g., phenyl), or R²⁹-substituted or unsubstituted 5 to 10 membered heteroaryl (e.g., 5 to 9 membered or 5 to 6 membered).

In embodiments, R²⁸ is R²⁹-substituted or unsubstituted alkyl (e.g., C₁-C₈ alkyl, C₃-C₆ alkyl, or C₁-C₄ alkyl). In embodiments, R²⁸ is R²⁹-substituted alkyl (e.g., C₁-C₈ alkyl, C₁-C₆ alkyl, or C₁-C₄ alkyl). In embodiments, R²⁸ is an unsubstituted alkyl (e.g., C₁-C₈ alkyl, C₁-C₆ alkyl, or C₁-C₄ alkyl).

In embodiments, R²⁸ is R²⁹-substituted or unsubstituted heteroalkyl (e.g., 2 to 8 membered heteroalkyl, 2 to 6 membered heteroalkyl, or 2 to 4 membered heteroalkyl). In embodiments, R²⁸ is R²⁹-substituted heteroalkyl (e.g., 2 to 8 membered heteroalkyl, 2 to 6 membered heteroalkyl, or 2 to 4 membered heteroalkyl). In embodiments, R²⁸ is an unsubstituted heteroalkyl (e.g., 2 to 8 membered heteroalkyl, 2 to 6 membered heteroalkyl, or 2 to 4 membered heteroalkyl).

In embodiments, R²⁸ is R²⁹-substituted or unsubstituted cycloalkyl (e.g., C₃-C₈ cycloalkyl, C₃-C₆ cycloalkyl, or C₅-C₆ cycloalkyl). In embodiments, R²⁸ is R²⁹-substituted cycloalkyl (e.g., C₃-C₈ cycloalkyl, C₃-C₆ cycloalkyl, or C₅-C₆ cycloalkyl). In embodiments, R²⁸ is an unsubstituted cycloalkyl (e.g., C₃-C₈ cycloalkyl, C₃-C₆ cycloalkyl, or C₅-C₆ cycloalkyl).

In embodiments, R²⁸ is R²⁹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁸ is R²⁹-substituted heterocycloalkyl (e.g., 3 to 8 membered heterocycloalkyl, 3 to 6 membered heterocycloalkyl, or 5 to 6 membered heterocycloalkyl). In embodiments, R²⁸ is an unsubstituted heterocycloalkyl (e.g., 3 to 8 membered heterocycloalkyl, 3 to 6 membered heterocycloalkyl, or 5 to 6 membered heterocycloalkyl).

In embodiments, R²⁸ is R²⁹-substituted or unsubstituted aryl (e.g., C₆-C₁₀ aryl, C₁₀ aryl, or phenyl). In embodiments, R²⁸ is R²⁹-substituted aryl (e.g., C₆-C₁₀ aryl, C₁₀ aryl, or phenyl). In embodiments, R²⁸ is an unsubstituted aryl (e.g., C₆-C₁₀ aryl, C₁₀ aryl, or phenyl).

In embodiments, R²⁸ is R²⁹-substituted or unsubstituted heteroaryl (e.g., 5 to 10 membered heteroaryl, 5 to 9 membered heteroaryl, or 5 to 6 membered heteroaryl). In embodiments, R²⁸ is R²⁹-substituted heteroaryl (e.g., 5 to 10 membered heteroaryl, 5 to 9 membered heteroaryl, or 5 to 6 membered heteroaryl). In embodiments, R²⁸ is an unsubstituted heteroaryl (e.g., 5 to 10 membered heteroaryl, 5 to 9 membered heteroaryl, or 5 to 6 membered heteroaryl).

In embodiments, R²⁶ is independently —N₃, —COOR^{26C}, —CONR^{26A}R^{26B}, —NR^{26D}C(O)NR^{26A}R^{26B}, —NR^{26A}C(O)OR^{26C}, or —C(O)R^{26C}. In embodiments, R²⁶ is independently —N₃. In embodiments, R²⁶ is independently —COOR^{26C}. In embodiments, R²⁶ is independently —CONR^{26A}R^{26B}. In embodiments, R²⁶ is independently —NR^{26D}C(O)NR^{26A}R^{26B}. In embodiments, R²⁶ is independently —NR^{26A}C(O)OR^{26C}. In embodiments, R²⁶ is independently —C(O)R^{26C}. In embodiments, R²⁶ is independently —N₃, —COOR^{26C}, —CONR^{26A}R^{26B}, —NR^{26D}C(O)NR^{26A}R^{26B}, —NR^{26A}C(O)OR^{26C}, or —C(O)R^{26C}. In embodiments, R²⁶ is independently —COOR^{26C}, —NR^{26A}C(O)OR^{26C}, or —C(O)R^{26C}. In embodiments, R²⁶ is independently —COOR^{26C}. In embodiments, R²⁶ is independently —NR^{26A}C(O)OR^{26C}. In embodiments, R²⁶ is independently —C(O)R^{26C}.

R^{26A} is independently hydrogen, —CX^{26A}₃, —CN, —COOH, —CONH₂, —CHX^{26A}₂, —CH₂X^{26A}, R^{27A}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{27A}-substituted or unsubstituted 2 to 8 membered heteroalkyl (e.g., 2 to 6 membered, 4 to 6 membered, or 4 to 5 membered), R^{27A}-substituted or unsubstituted C₃-C₈ cycloalkyl (e.g., C₃-C₆, C₄-C₆, or C₅-C₆), R^{27A}-substituted or unsubstituted 3 to 6 membered heterocycloalkyl (e.g., 4 to 6 membered, 4 to 5 membered, or to 6 membered), R^{27A}-substituted or unsubstituted C₆-C₁₀ aryl (e.g., phenyl), or R^{27A}-substituted or unsubstituted 5 to 10 membered heteroaryl (e.g., 5 to 9 membered or 5 to 6 membered). X^{26A} is —F, —Cl, —Br, or —I. In embodiments, R^{26A} is independently hydrogen. In embodiments, R^{26A} is independently methyl. In embodiments, R^{26A} is independently ethyl. In embodiments, R^{26A} is independently hydrogen, —CX^{26A}₃, —CN, —COOH, —CONH₂, R^{27A}-substituted or unsubstituted C₁-C₈ alkyl, R^{27A}-substituted or unsubstituted 2 to 8 membered heteroalkyl, R^{27A}-substituted or unsubstituted C₃-C₈ cycloalkyl, R^{27A}-substituted or unsubstituted 3 to 8 membered heterocycloalkyl, R^{27A}-substituted or unsubstituted C₆-C₁₀ aryl, or R^{27A}-substituted or unsubstituted 5 to 10 membered heteroaryl. In embodiments, R^{26A} is independently unsubstituted C₁-C₄ alkyl or R^{27A}-substituted or unsubstituted phenyl. In embodiments, R^{26A} is independently unsubstituted C₁-C₄ alkyl. In embodiments, R^{26A} is independently R^{27A}-substituted or unsubstituted phenyl. In embodiments, R^{26A} is independently hydroxyl-substituted phenyl. In embodiments, R^{26A} is independently unsubstituted phenyl. In embodiments, R^{26A} is independently oxo, halogen, —CX^{26A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{26A}₃, —OCHX^{26A}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{26A} and R^{26B} substituents bonded to the same nitrogen atom may optionally be joined to form a R^{27A}-substituted or unsubstituted 3 to 6 membered heterocycloalkyl (e.g., 4 to 6 membered, 4 to 5 membered, or 5 to 6 membered) or R^{27A}-substituted or unsubstituted 5 to 6 membered heteroaryl. In embodiments, R^{26A} and R^{26B} substituents bonded to the same nitrogen atom may optionally be joined to form an R^{27A}-substituted or unsubstituted 3 to 8 membered heterocycloalkyl or R^{27A}-substituted or unsubstituted 5 to 10 membered heteroaryl. In embodiments, R^{26A} and R^{26B} substituents bonded to the same nitrogen atom may optionally be joined to form an unsubstituted 3 to 8 membered heterocycloalkyl or unsubstituted 5 to 10 membered heteroaryl. In embodiments, R^{26A} and R^{26B} substituents bonded to the same nitrogen atom may optionally be joined to form a R^{27A}-substituted or unsubstituted piperazinyl. In embodiments, R^{26A} and R^{26B} substituents bonded to the same nitrogen atom may optionally be joined to form a R^{27A}-substituted or unsubstituted piperidinyl. In embodiments, R^{26A} and R^{26B} substituents bonded to the same nitrogen atom may optionally be joined to form a R^{27A}-substituted or unsubstituted pyrrolidinyl. In embodiments, R^{26A} and R^{26B} substituents bonded to the same nitrogen atom may optionally be joined to form a R^{27A}-substituted or unsubstituted azetidinyl. In embodiments, R^{26A} and R^{26B} substituents bonded to the same nitrogen atom may optionally be joined to form a R^{27A}-substituted or unsubstituted morpholinyl. In embodiments, R^{26A} and R^{26B} substituents bonded to the same nitrogen atom may optionally be joined to form a R^{27A}-substituted or unsubstituted aziridinyl.

R^{27A} is independently oxo, halogen, —CX^{27A}₃, —CHX^{27A}₂, —CH₂X^{27A}, —OCX^{27A}₃, —OCH₂X^{27A}, —OCHX^{27A}₂, —CN, —N₃, —SR^{27AD}, —ONR^{27AA}R^{27AB}, —NHC(O)NR^{27AA}R^{27AB}, —NR^{27AA}R^{27AB}, —C(O)R^{27AC}, —C(O)OR^{27AC}, —C(O)NR^{27AA}R^{27AB}, —OR^{27AD}, —NR^{27AA}C(O)R^{27AC}, —NR^{27AA}C(O)OR^{27AC}, —NR^{27AA}OR^{27AC}, —N₃, R^{28A}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28A}-substituted or unsubstituted 2 to 8 membered heteroalkyl (e.g., 2 to 6 membered, 4 to 6 membered, or 4 to 5 membered), R^{28A}-substituted or unsubstituted C₃-C₈ cycloalkyl (e.g., C₃-C₆, C₄-C₆, or C₅-C₆), R^{28A}-substituted or unsubstituted 3 to 6 membered heterocycloalkyl (e.g., 4 to 6 membered, 4 to 5 membered, or 5 to 6 membered), R^{28A}-substituted or unsubstituted C₆-C₁₀ aryl (e.g., phenyl), or R^{28A}-substituted or unsubstituted 5 to 10 membered heteroaryl (e.g., 5 to 9 membered or 5 to 6 membered). X^{27A} is —F, —Cl, —Br, or —I.

In embodiments, R^{27A} is independently oxo, halogen, —CX^{27A}₃, —CHX^{27A}₂, —CH₂X^{27A}, —OCX^{27A}₃, —OCH₂X^{27A}, —OCHX^{27A}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, R^{28A}-substituted or unsubstituted C₁-C₈ alkyl (e.g., C₁-C₆, C₁-C₄, C₃-C₂, C₂-C₆ alkenyl, C₂-C₄ alkenyl, C₂-C₆ alkynyl, or C₂-C₄ alkynyl), R^{28A}-substituted or unsubstituted 2 to 8 membered heteroalkyl (e.g., 2 to 6 membered, 4 to 6 membered, or 4 to 5 membered), R^{28A}-substituted or unsubstituted C₃-C₈ cycloalkyl (e.g., C₃-C₆, C₄-C₆, or C₅-C₆), R^{28A}-substituted or unsubstituted 3 to 6 membered heterocycloalkyl (e.g., 4 to 6 membered, 4 to 5 membered, or 5 to 6 membered), R^{28A}-substituted or unsubstituted C₆-C₁₀ aryl (e.g., phenyl), or R^{28A}-substituted or unsubstituted 5 to 10 membered heteroaryl (e.g., 5 to 9 membered or 5 to 6 membered). X^{27A} is —F, —Cl, —Br, or —I. In embodiments, R^{27A} is independently oxo, halogen, —CX^{27A}₃, —CHX^{27A}₂, —CH₂X^{27A}, —OCX^{27A}₃, —OCH₂X^{27A}, —OCHX^{27A}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{28A} is independently oxo, halogen, —CX^{28A}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28A}₃, —OCHX^{28A}₂, —N₃, R^{29A}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{29A}-substituted or unsubstituted 2 to 8 membered heteroalkyl (e.g., 2 to 6 membered, 4 to 6 membered, or 4 to 5 membered), R^{29A}-substituted or unsubstituted C₃-C₈ cycloalkyl (e.g., C₃-C₆, C₄-C₆, or C₅-C₆), R^{29A}-substituted or unsubstituted 3 to 6 membered heterocycloalkyl (e.g., 4 to 6 membered, 4 to 5 membered, or 5 to 6 membered), R^{29A}-substituted or unsubstituted phenyl, or R^{29A}-substituted or unsubstituted 5 to 6 membered heteroaryl. X^{28A} is —F, —Cl, —Br, or —I. In embodiments, R^{28A} is independently oxo, halogen, —CX₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28A}₃, —OCHX^{28A}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{26B} is independently hydrogen, —CX^{26B}₃, —CN, —N₃, —COOH, —CONH₂, —CHX^{26B}₂, —CH₂X^{26B}, R^{27B}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{27B}-substituted or unsubstituted 2 to 8 membered heteroalkyl (e.g., 2 to 6 membered, 4 to 6 membered, or 4 to 5 membered), R^{27B}-substituted or unsubstituted C₃-C₈ cycloalkyl (e.g., C₃-C₆, C₄-C₆, or C₅-C₆), R^{27B}-substituted or unsubstituted 3 to 6 membered heterocycloalkyl (e.g., 4 to 6 membered, 4 to 5 membered, or 5 to 6 membered), R^{27B}-substituted or unsubstituted C₆-C₁₀ aryl (e.g., phenyl), or R^{27B}-substituted or unsubstituted 5 to 10 membered heteroaryl (e.g., 5 to 9 membered or 5 to 6 membered). X^{26B} is —F, —Cl, —Br, or —I. In embodiments, R^{26B} is independently hydrogen. In embodiments, R^{26B} is independently 26B 26B methyl. In embodiments, R^{26B} is independently ethyl. In embodiments, R^{26B} is independently hydrogen, —CX₃, —CN, —COOH, —CONH₂, R^{27B}-substituted or unsubstituted C₁-C₈ alkyl, R^{27B}-substituted or unsubstituted 2 to 8 membered heteroalkyl, R^{27B}-substituted or unsubstituted C₃-C₈ cycloalkyl, R^{27B}-substituted or unsubstituted 3 to 8 membered heterocycloalkyl, R^{27B}-substituted or unsubstituted C₆-C₁₀ aryl, or R^{27B}-substituted or unsubstituted 5 to 10 membered heteroaryl. In embodiments, R^{26B} is independently unsubstituted C₁-C₄ alkyl or R^{27B}-substituted or unsubstituted phenyl. In embodiments, R^{26B} is independently unsubstituted C₁-C₄ alkyl. In embodiments, R^{26B} is independently R^{27B}-substituted or unsubstituted phenyl. In embodiments, R^{26B} is independently hydroxyl-substituted phenyl. In embodiments, R^{26B} is independently unsubstituted phenyl. In embodiments, R^{26B} is independently oxo, halogen, —CX^{26B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{26B}₃, —OCHX^{26B}₂, unsubstituted C₁-C₈ alkyl (e.g., C₁-C₆, C₁-C₄, or C₃-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{26A} and R^{26B} substituents bonded to the same nitrogen atom may optionally be joined to form a R^{27B}-substituted or unsubstituted 3 to 6 membered heterocycloalkyl (e.g., 4 to 6 membered, 4 to 5 membered, or 5 to 6 membered) or R^{27B}-substituted or unsubstituted 5 to 6 membered heteroaryl. In embodiments, R^{26A} and R^{26B} substituents bonded to the same nitrogen atom may optionally be joined to form an R^{27B}-substituted or unsubstituted 3 to 8 membered heterocycloalkyl or R^{27B}-substituted or unsubstituted 5 to 10 membered heteroaryl. In embodiments, R^{26A} and R^{26B} substituents bonded to the same nitrogen atom may optionally be joined to form an unsubstituted 3 to 8 membered heterocycloalkyl or unsubstituted 5 to 10 membered heteroaryl. In embodiments, R^{26A} and R^{26B} substituents bonded to the same nitrogen atom may optionally be joined to form a R^{27B}-substituted or unsubstituted piperazinyl. In embodiments, R^{26A} and R^{27B}-substituents bonded to the same nitrogen atom may optionally be joined to form a R^{27B}-substituted or unsubstituted piperidinyl. In embodiments, R^{26A} and R^{26B} substituents bonded to the same nitrogen atom may optionally be joined to form a R^{27B}-substituted or unsubstituted pyrrolidinyl. In embodiments, R^{26A} and R^{26B} substituents bonded to the same nitrogen atom may optionally be joined to form a R^{27B}-substituted or unsubstituted azetidinyl. In embodiments, R^{26A} and R^{26B} substituents bonded to the same nitrogen atom may optionally be joined to form a R^{27B}-substituted or unsubstituted morpholinyl. In embodiments, R^{26A} and R^{26B} substituents bonded to the same nitrogen atom may optionally be joined to form a R^{27B}-substituted or unsubstituted aziridinyl.

R^{27B} is independently oxo, halogen, —CX²⁷—CHX^{27B}₂, —CH₂X^{27B}, —OCX^{27B}₃, —OCH₂X^{27B}, —OCHX^{27B}₂, —CN, —N₃, —SR^{27BD}, —ONR^{27BA}R^{27BB}, —NHC(O)NR^{27BA}R^{27BB}, —NR^{27BA}R^{27BB}, —C(O)R^{27BC}, —C(O)OR^{27BC}, —C(O)NR^{27BA}R^{27BB}, —OR^{27BD}, —NR^{27BA}C(O)R^{27BC}, —NR^{27BA}C(O)OR^{27BC}, —NR^{27BA}OR^{27BC}, —N₃, R^{28B}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28B}-substituted or unsubstituted 2 to 8 membered heteroalkyl (e.g., 2 to 6 membered, 4 to 6 membered, or 4 to 5 membered), R^{28B}-substituted or unsubstituted C₃-C₈ cycloalkyl (e.g., C₃-C₆, C₄-C₆, or C₅-C₆), R^{28B}-substituted or unsubstituted 3 to 6 membered heterocycloalkyl (e.g., 4 to 6 membered, 4 to 5 membered, or 5 to 6 membered), R^{28B}-substituted or unsubstituted C₆-C₁₀ aryl (e.g., phenyl), or R^{28B}-substituted or unsubstituted 5 to 10 membered heteroaryl (e.g., 5 to 9 membered or 5 to 6 membered). X^{27B} is —F, —Cl, —Br, or —I. In embodiments, R^{27B} is independently oxo, halogen, —CX²⁷—CHX^{27B}₂, —CH₂X^{27B}, —OCX^{27B}₃, —OCH₂X^{27B}, —OCHX^{27B}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{27B} is independently oxo, halogen, —CX^{27B}₃, —CHX^{27B}₂, —CH₂X^{27B}, —OCX^{27B}₃, —OCH₂X^{27B}, —OCHX^{27B}2, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, R^{28B}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28B}-substituted or unsubstituted 2 to 8 membered heteroalkyl (e.g., 2 to 6 membered, 4 to 6 membered, or 4 to 5 membered), R^{28B}-substituted or unsubstituted C₃-C₈ cycloalkyl (e.g., C₃-C₆, C₄-C₆, or C₅-C₆), R^{28B}-substituted or unsubstituted 3 to 6 membered heterocycloalkyl (e.g., 4 to 6 membered, 4 to 5 membered, or 5 to 6 membered), R^{28B}-substituted or unsubstituted C₆-C₁₀ aryl (e.g., phenyl), or R^{28B}-substituted or unsubstituted 5 to 10 membered heteroaryl (e.g., 5 to 9 membered or 5 to 6 membered). X^{27B} is —F, —Cl, —Br, or —I. In embodiments, R^{27B} is independently oxo, halogen, —CX²⁷—CHX^{27B}₂, —CH₂X^{27B}, —OCX^{27B}₃, —OCH₂X^{27B}, —OCHX^{27B}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{28B} is independently oxo, halogen, —CX^{28B}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28B}₃, —OCHX^{28B}₂, —N₃, R^{29B}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{29B}-substituted or unsubstituted 2 to 8 membered heteroalkyl (e.g., 2 to 6 membered, 4 to 6 membered, or 4 to 5 membered), R^{29B}-substituted or unsubstituted C₃-C₈ cycloalkyl (e.g., C₃-C₆, C₄-C₆, or C₅-C₆), R^{29B}-substituted or unsubstituted 3 to 6 membered heterocycloalkyl (e.g., 4 to 6 membered, 4 to 5 membered, or 5 to 6 membered), R^{29B}-substituted or unsubstituted phenyl, or R^{29B}-substituted or unsubstituted 5 to 6 membered heteroaryl. X^{28B} is —F, —Cl, —Br, or —I. In embodiments, R^{28B} is independently oxo, halogen, —CX²⁸—CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28B}₃, —OCHX^{28B}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{26C} is independently hydrogen, —CX^{26C}₃, —CN, —N₃, —COOH, —CONH₂, —CHX^{26C}₂, —CH₂X^{26C}, R^{27C}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{27C}-substituted or unsubstituted 2 to 8 membered heteroalkyl (e.g., 2 to 6 membered, 4 to 6 membered, or 4 to 5 membered), R^{27C}-substituted or unsubstituted C₃-C₈ cycloalkyl (e.g., C₃-C₆, C₄-C₆, or C₅-C₆), R^{27C}-substituted or unsubstituted 3 to 6 membered heterocycloalkyl (e.g., 4 to 6 membered, 4 to 5 membered, or 5 to 6 membered), R^{27C}-substituted or unsubstituted C₆-C₁₀ aryl (e.g., phenyl), or R^{27C}-substituted or unsubstituted 5 to 10 membered heteroaryl (e.g., 5 to 9 membered or 5 to 6 membered). X^{26C} is —F, —Cl, —Br, or —I. In embodiments, R^{26C} is independently hydrogen. In embodiments, R^{26C} is R^{27C}-substituted or unsubstituted alkyl (e.g., C₁-C₈ alkyl, C₁-C₆ alkyl, or C₁-C₄ alkyl). In embodiments, R^{26C} is R^{27C}-substituted alkyl (e.g., C₁-C₈ alkyl, C₁-C₆ alkyl, or C₁-C₄ alkyl). In embodiments, R¹ is an unsubstituted alkyl (e.g., C₁-C₈ alkyl, C₁-C₆ alkyl, or C₁-C₄ alkyl). In embodiments, R^{26C} is independently methyl. In embodiments, R^{26C} is independently ethyl.

In embodiments, R^{26C} is independently hydrogen, —CX^{26C}₃, —CN, —N₃, —COOH, —CONH₂, R^{27C}-substituted or unsubstituted C₁-C₈ alkyl, R^{27C}-substituted or unsubstituted 2 to 8 membered heteroalkyl, R^{27C}-substituted or unsubstituted C₃-C₈ cycloalkyl, R^{27C}-substituted or unsubstituted 3 to 8 membered heterocycloalkyl, R^{27C}-substituted or unsubstituted C₆-C₁₀ aryl, or R^{27C}-substituted or unsubstituted 5 to 10 membered heteroaryl. In embodiments, R^{26C} is independently unsubstituted C₁-C₄ alkyl or R^{27C}-substituted or unsubstituted phenyl. In embodiments, R^{26C} is independently unsubstituted C₁-C₄ alkyl. In embodiments, R^{26C} is independently R^{27C}-substituted or unsubstituted phenyl. In embodiments, R^{26C} is independently hydroxyl-substituted phenyl. In embodiments, R^{26C} is independently unsubstituted phenyl. In embodiments, R^{26C} is independently oxo, halogen, —CX^{26C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{26C}₃, —OCHX^{26C}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{27C} is independently oxo, halogen, —CX^{27C}₃, —CHX^{27C}₂, —CH₂X^{27C}, —OCX^{27C}₃, —OCH₂X^{27C}, —OCHX^{27C}₂, —CN, —N₃, —SR^{27CD}, —ONR^{27CA}R^{27CB}, —NHC(O)NR^{27CA}R^{27CB}, —NR^{27CA}R^{27CB}, —C(O)R^{2?cc}, —C(O)OR^{27CC}, —C(O)NR^{27CA}R^{27CB}, —OR^{27CD}, —NR^{27CA}C(O)R^{27CC}, —NR^{27CA}C(O)OR^{27CC}, —NR^{27CA}OR^{27CC}, —N₃, R^{28C}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28C}-substituted or unsubstituted 2 to 8 membered heteroalkyl (e.g., 2 to 6 membered, 4 to 6 membered, or 4 to 5 membered), R^{28C}-substituted or unsubstituted C₃-C₈ cycloalkyl (e.g., C₃-C₆, C₄-C₆, or C₅-C₆), R^{28C}-substituted or unsubstituted 3 to 6 membered heterocycloalkyl (e.g., 4 to 6 membered, 4 to 5 membered, or 5 to 6 membered), R^{28C}-substituted or unsubstituted C₆-C₁₀ aryl (e.g., phenyl), or R^{28C}-substituted or unsubstituted 5 to 10 membered heteroaryl (e.g., 5 to 9 membered or 5 to 6 membered). X^{27C} is —F, —Cl, —Br, or —I. In embodiments, R^{27C} is independently oxo, halogen, —CX^{27C}₃, —CHX^{27C}₂, —CH₂X^{27C}, —OCX^{27C}₃, —OCH₂X^{27C}, —OCHX^{27C}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{27C} is independently oxo, halogen, —CX^{27C}₃, —CHX^{27C}₂, —CH₂X^{27C}, —OCX^{27C}₃, —OCH₂X^{27C}, —OCHX^{27C}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, R^{28C}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28C}-substituted or unsubstituted 2 to 8 membered heteroalkyl (e.g., 2 to 6 membered, 4 to 6 membered, or 4 to 5 membered), R^{28C}-substituted or unsubstituted C₃-C₈ cycloalkyl (e.g., C₃-C₆, C₄-C₆, or C₅-C₆), R^{28C}-substituted or unsubstituted 3 to 6 membered heterocycloalkyl (e.g., 4 to 6 membered, 4 to 5 membered, or 5 to 6 membered), R^{28C}-substituted or unsubstituted C₆-C₁₀ aryl (e.g., phenyl), or R^{28C}-substituted or unsubstituted 5 to 10 membered heteroaryl (e.g., 5 to 9 membered or 5 to 6 membered). X^{27C} is —F, —Cl, —Br, or —I. In embodiments, R^{27C} is independently oxo, halogen, —CX^{27C}₃, —CHX^{27C}₂, —CH₂X^{27C}, —OCX^{27C}₃, —OCH₂X^{27C}, —OCHX^{27C}₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{28C} is independently oxo, halogen, —CX^{28C}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28C}₃, —OCHX^{28C}2, R^{29C}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{29C}-substituted or unsubstituted 2 to 8 membered heteroalkyl (e.g., 2 to 6 membered, 4 to 6 membered, or 4 to 5 membered), R^{29C}-substituted or unsubstituted C₃-C₈ cycloalkyl (e.g., C₃-C₆, C₄-C₆, or C₅-C₆), R^{29C}-substituted or unsubstituted 3 to 6 membered heterocycloalkyl (e.g., 4 to 6 membered, 4 to 5 membered, or 5 to 6 membered), R^{29C}-substituted or unsubstituted phenyl, or R^{29C}-substituted or unsubstituted 5 to 6 membered heteroaryl. X^{28C} is —F, —Cl, —Br, or —I. In embodiments, R^{28C} is independently oxo, halogen, —CX₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28C}₃, —OCHX^{28C}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{26D} is independently hydrogen, —CX^{26D}₃, —CN, —N₃, —COOH, —CONH₂, —CHX^{26D}₂, —CH₂X^{26D}, R^{27D}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{27D}-substituted or unsubstituted 2 to 8 membered heteroalkyl (e.g., 2 to 6 membered, 4 to 6 membered, or 4 to 5 membered), R^{27D}-substituted or unsubstituted C₃-C₈ cycloalkyl (e.g., C₃-C₆, C₄-C₆, or C₅-C₆), R^{27D}-substituted or unsubstituted 3 to 6 membered heterocycloalkyl (e.g., 4 to 6 membered, 4 to 5 membered, or 5 to 6 membered), R^{27D}-substituted or unsubstituted C₆-C₁₀ aryl (e.g., phenyl), or R^{27D}-substituted or unsubstituted 5 to 10 membered heteroaryl (e.g., 5 to 9 membered or 5 to 6 membered). X^{26D} is —F, —Cl, —Br, or —I In embodiments, R^{26D} is independently hydrogen. In embodiments, R^{26D} is independently methyl. In embodiments, R^{26D} is independently ethyl. In embodiments, R^{26D} is independently hydrogen, —CX^{26D}₃, —CN, —N₃, —COOH, —CONH₂, R^{27D}-substituted or unsubstituted C₁-C₈ alkyl, R^{27D}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27D}-substituted or unsubstituted C₆-C₁₀ aryl, or R^{27D}-substituted or unsubstituted 5 to 10 membered heteroaryl. In embodiments, R^{26D} is independently unsubstituted C₁-C₄ alkyl or R^{27D}-substituted or unsubstituted phenyl. In embodiments, R^{26D} is independently unsubstituted C₁-C₄ alkyl. In embodiments, R^{26D} is independently R^{27D}-substituted or unsubstituted phenyl. In embodiments, R^{26D} is independently hydroxyl-substituted phenyl. In embodiments, R^{26D} is independently unsubstituted phenyl. In embodiments, R^{26D} is independently oxo, halogen, —CX^{26D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{26D}₃, —OCHX^{26D}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{27D} is independently oxo, halogen, —CX^{27D}₃, —CHX^{27D}₂, —CH₂X^{27D}, —OCX^{27D}₃, —OCH₂X^{27D}, —OCHX^{27D}₂, —CN, —SR^{27DD}, —ONR^{27DA}R^{27DB}, —NHC(O)NR^{27DA}R^{27DB}, —NR^{27DA}R^{27DB}, —C(O)R^{27DC}, —C(O)OR^{27DC}, —C(O)NR^{27DA}R^{27DB}, —OR^{27DD}, —NR^{27DA}C(O)R^{27DC}, —NR^{27DA}C(O)OR^{27DC}, —NR^{27DA}OR^{27DC}, —N₃, R^{28D}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28D}-substituted or unsubstituted 2 to 8 membered heteroalkyl (e.g., 2 to 6 membered, 4 to 6 membered, or 4 to 5 membered), R^{28D}-substituted or unsubstituted C₃-C₈ cycloalkyl (e.g., C₃-C₆, C₄-C₆, or C₅-C₆), R^{28D}-substituted or unsubstituted 3 to 6 membered heterocycloalkyl (e.g., 4 to 6 membered, 4 to 5 membered, or 5 to 6 membered), R^{28D}-substituted or unsubstituted C₆-C₁₀ aryl (e.g., phenyl), or R^{28D}-substituted or unsubstituted 5 to 10 membered heteroaryl (e.g., 5 to 9 membered or 5 to 6 membered). X^{27D} is —F, —Cl, —Br, or —I In embodiments, R^{27D} is independently oxo, halogen, —CX^{27D}₃, —CHX^{27D}₂, —CH₂X^{27D}, —OCX^{27D}₃, —OCH₂X^{27D}, —OCHX^{27D}₂, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₃-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

In embodiments, R^{27D} is independently oxo, halogen, —CX^{27D}₃, —CHX^{27D}₂, —CH₂X^{27D}, —OCX^{27D}₃, —OCH₂X^{27D}, —OCHX^{27D}₂, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, R^{28D}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{28D}-substituted or unsubstituted 2 to 8 membered heteroalkyl (e.g., 2 to 6 membered, 4 to 6 membered, or 4 to 5 membered), R^{28D}-substituted or unsubstituted C₃-C₈ cycloalkyl (e.g., C₃-C₆, C₄-C₆, or C₅-C₆), R^{28D}-substituted or unsubstituted 3 to 6 membered heterocycloalkyl (e.g., 4 to 6 membered, 4 to 5 membered, or 5 to 6 membered), R^{28D}-substituted or unsubstituted C₆-C₁₀ aryl (e.g., phenyl), or R^{28D}-substituted or unsubstituted 5 to 10 membered heteroaryl (e.g., 5 to 9 membered or 5 to 6 membered). X^{27D} is —F, —Cl, —Br, or —I. In embodiments, R^{27D} is independently oxo, halogen, —CX^{27D}₃, —CHX^{27D}₂, —CH₂X^{27D}, —OCX^{27D}₃, —OCH₂X^{27D}, —OCHX^{27D}₂, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{28D} is independently oxo, halogen, —CX^{28D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28D}₃, —OCHX^{28D}₂, R^{29D}-substituted or unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), R^{29D}-substituted or unsubstituted 2 to 8 membered heteroalkyl (e.g., 2 to 6 membered, 4 to 6 membered, or 4 to 5 membered), R^{29D}-substituted or unsubstituted C₃-C₈ cycloalkyl (e.g., C₃-C₆, C₄-C₆, or C₅-C₆), R^{29D}-substituted or unsubstituted 3 to 6 membered heterocycloalkyl (e.g., 4 to 6 membered, 4 to 5 membered, or 5 to 6 membered), R^{29D}-substituted or unsubstituted phenyl, or R^{29D}-substituted or unsubstituted 5 to 6 membered heteroaryl. X^{28D} is —F, —Cl, —Br, or —I. In embodiments, R^{28D} is independently oxo, halogen, —CX^{28D}₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCX^{28D}₃, —OCHX^{28D}₂, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R²⁹, R^{29A}, R^{29B}, R^{29C}, and R^{29D} are independently oxo, halogen, —CF₃, —CHF₂, —CH₂F, —OCF₃, —OCH₂F, —OCHF₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —N₃, unsubstituted C₁-C₈ alkyl (e.g., C₁-C₆, C₁-C₄, C₁-C₂, C₂-C₆ alkenyl, C₂-C₄ alkenyl, C₂-C₆ alkynyl, or C₂-C₄ alkynyl), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl.

R^{27AA}, R^{27AB}, R^{27AC}, R^{27AD}, R^{27BA}, R^{27BB}, R^{27BC}, R^{27BD}, R^{27CA}, R^{27CB}, R^{27CC}, R^{27CD}, R^{27DA}, R^{27DB}, R^{27DC}, and R^{27DD} are independently hydrogen, oxo, halogen, —CF₃, —CHF₂, —CH₂F, —OCF₃, —OCH₂F, —OCHF₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl. In embodiments, R^{27AA}, R^{27AB}, R^{27AC}, R^{27AD}, R^{27BA}, R^{27BB}, R^{27BC}, R^{27BD}, R^{27CA}, R^{27CB}, R^{27CC}, R^{27CD}, R^{27DA}, R^{27DB}, R^{27DC}, and R^{27DD} are independently oxo, halogen, —CF₃, —CHF₂, —CH₂F, —OCF₃, —OCH₂F, —OCHF₂, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, unsubstituted C₁-C₈ alkyl (e.g., C₁-C₆, C₁-C₄, or C₁-C₂), unsubstituted 2 to 8 membered heteroalkyl (e.g., 2 to 6 membered, 4 to 6 membered, or 4 to 5 membered), unsubstituted C₃-C₈ cycloalkyl (e.g., C₃-C₆, C₄-C₆, or C₅-C₆), unsubstituted 3 to 6 membered heterocycloalkyl (e.g., 4 to 6 membered, 4 to 5 membered, or 5 to 6 membered), unsubstituted phenyl, or unsubstituted 5 to 6 membered heteroaryl. In embodiments, R^{27AA} and R^{27AB} substituents bonded to the same nitrogen atom may optionally be joined to form an unsubstituted heterocycloalkyl (e.g., 3 to 8 membered, 4 to 6 membered, 4 to 5 membered, or 5 to 6 membered) or unsubstituted heteroaryl (e.g., 5 to membered, 5 to 9 membered, or 5 to 6 membered). In embodiments, R^{27AA} and R^{27AB} substituents bonded to the same nitrogen atom may optionally be joined to form an unsubstituted piperazinyl, unsubstituted piperidinyl, unsubstituted pyrrolidinyl, unsubstituted azetidinyl, unsubstituted morpholinyl, or unsubstituted aziridinyl. In embodiments, R^{27BA} and R^{27BB} substituents bonded to the same nitrogen atom may optionally be joined to form an unsubstituted heterocycloalkyl (e.g., 3 to 8 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{27BA} and R^{27BB} substituents bonded to the same nitrogen atom may optionally be joined to form an unsubstituted piperazinyl, unsubstituted piperidinyl, unsubstituted pyrrolidinyl, unsubstituted azetidinyl, unsubstituted morpholinyl, or unsubstituted aziridinyl. In embodiments, R^{27CA} and R^{27CB} substituents bonded to the same nitrogen atom may optionally be joined to form an unsubstituted heterocycloalkyl (e.g., 3 to 8 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R and R substituents bonded to the same nitrogen atom may optionally be joined to form an unsubstituted piperazinyl, unsubstituted piperidinyl, unsubstituted pyrrolidinyl, unsubstituted azetidinyl, unsubstituted morpholinyl, or unsubstituted aziridinyl. In embodiments, R^{27DA} and R^{27DB} substituents bonded to the same nitrogen atom may optionally be joined to form an unsubstituted heterocycloalkyl (e.g., 3 to 8 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{27DA} and R^{27DB} substituents bonded to the same nitrogen atom may optionally be joined to form an unsubstituted piperazinyl, unsubstituted piperidinyl, unsubstituted pyrrolidinyl, unsubstituted azetidinyl, unsubstituted morpholinyl, or unsubstituted aziridinyl.

In embodiments, the compound has the formula:

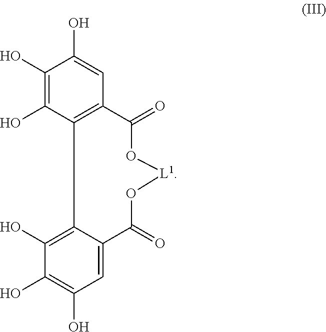

In embodiments, L¹ is unsubstituted butylene. In embodiments, L¹ is unsubstituted n-butylene. In embodiments, L¹ is substituted butylene. In embodiments, L¹ is R²⁶-substituted butylene. In embodiments, L¹ is —(CH₂)₀₋₄—O—(CH₂)₀₋₄—. In embodiments, L¹ is —(CH₂)₀₋₄—N(R²⁶)—(CH₂)₀₋₄—. In embodiments, L¹ is —(CH₂)₀₋₄—NH—(CH₂)₀₋₄—. In embodiments, L¹ is unsubstituted C₄-C₈ cyloalkylene.

In embodiments, the compound has the formula:

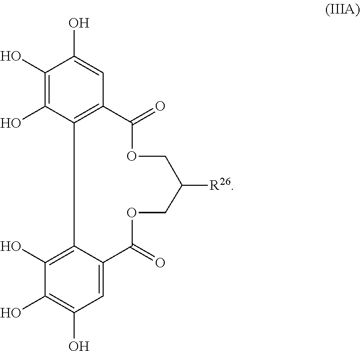

wherein R²⁶ is as described herein.

In embodiments, the compound has the formula:

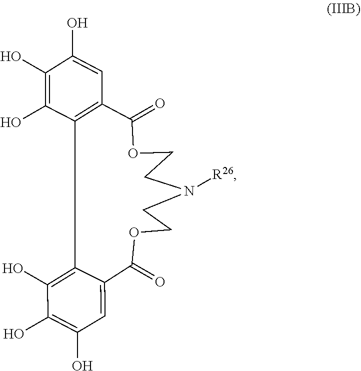

wherein R²⁶ is as described herein.

In embodiments, the compound has the formula:

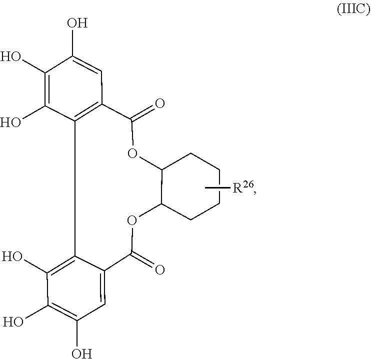

wherein R²⁶ is as described herein.

In embodiments, the compound has the formula:

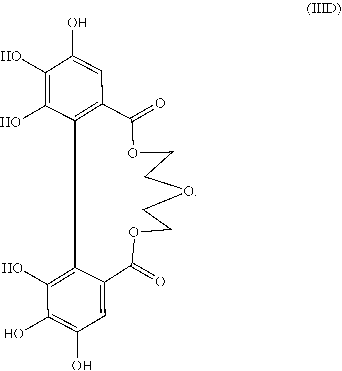 [SMILES: O=C1OCCOCCOC(=O)c2cc(O)c(O)c(O)c2-c2c1cc(O)c(O)c2O]

In embodiments, the compound has the formula:

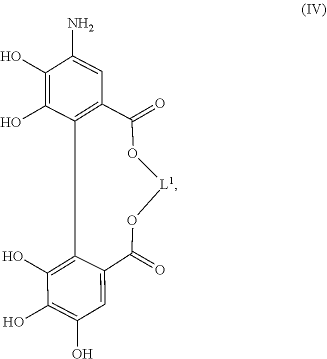

wherein L¹ is as described herein. In embodiments, L¹ is unsubstituted butylene. In embodiments, L¹ is unsubstituted n-butylene. In embodiments, L¹ is substituted butylene. In embodiments, L¹ is R²⁶-substituted butylene. In embodiments, L¹ is —(CH₂)₀₋₄—O—(CH₂)₀₋₄—. In embodiments, L¹ is —(CH₂)₀₋₄—N(R²⁶)—(CH₂)₀₋₄—. In embodiments, L¹ is (CH₂)₀₋₄—NH—(CH₂)₀₋₄—. In embodiments, L¹ is unsubstituted C₄-C₈ cyloalkylene.

In embodiments, the compound has the formula:

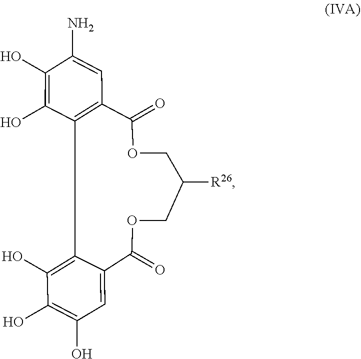

wherein R²⁶ is as described herein.

In embodiments, the compound has the formula:

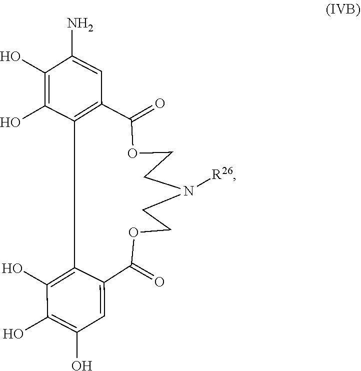

wherein R²⁶ is as described herein.

In embodiments, the compound has the formula:

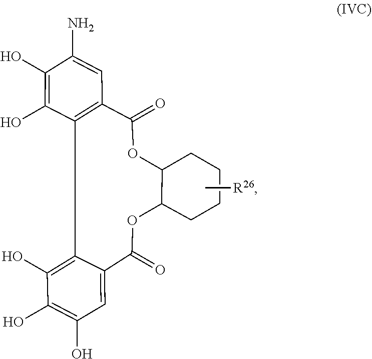

wherein R²⁶ is as described herein.

In embodiments, the compound has the formula:

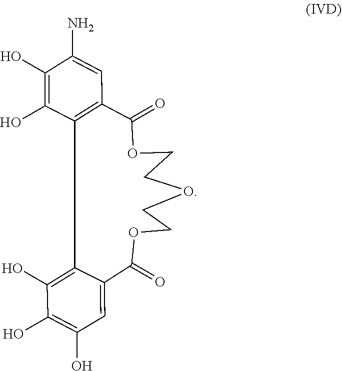 [SMILES: Nc1cc2c(c(O)c1O)-c1c(cc(O)c(O)c1O)C(=O)OCCOCCOC2=O]

In embodiments, the compound has the formula:

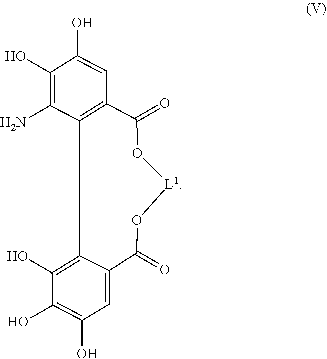

In embodiments, L¹ is unsubstituted butylene. In embodiments, L¹ is unsubstituted n-butylene. In embodiments, L¹ is substituted butylene. In embodiments, L¹ is R²⁶-substituted butylene. In embodiments, L¹ is —(CH₂)₀₋₄—O—(CH₂)₀₋₄—. In embodiments, L¹ is —(CH₂)₀₋₄—N(R²⁵)—(CH₂)₀₋₄—. In embodiments, L¹ is —(CH₂)₀₋₄—NH—(CH₂)₀₋₄—. In embodiments, L¹ is unsubstituted C₄-C₈ cyloalkylene.

In embodiments, the compound has the formula:

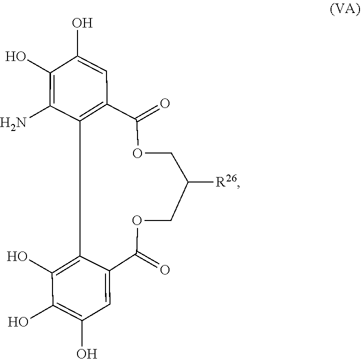

wherein R²⁶ is as described herein.

In embodiments, the compound has the formula:

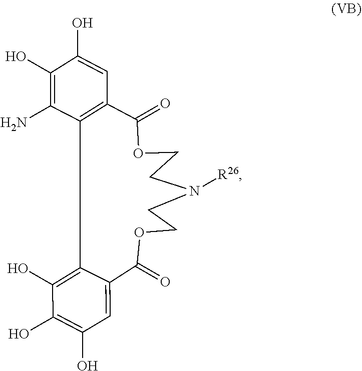

wherein R²⁶ is as described herein.

In embodiments, the compound has the formula:

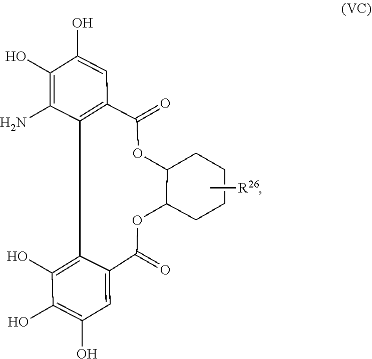

wherein R²⁶ is as described herein.

In embodiments, the compound has the formula:

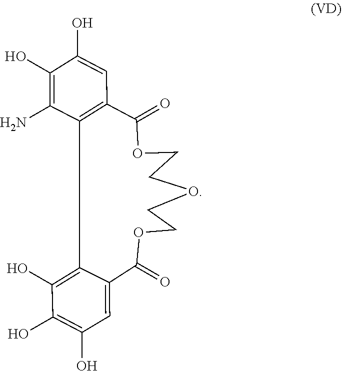 [SMILES: Nc1c(O)c(O)cc2c1-c1c(cc(O)c(O)c1O)C(=O)OCCOCCOC2=O]

In embodiments, R²⁶ is tert-butyloxycarbonyl. In embodiments, R²⁶ is —C(O)OC(CH₃)₃. In embodiments, R²⁶ is —NHC(O)OC(CH₃)₃. In embodiments, R²⁶ is —C(O)Ph. In embodiments, R²⁶ is

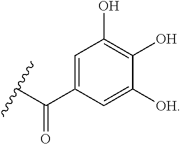 [SMILES: *C(=O)c1cc(O)c(O)c(O)c1]

In embodiments, R²⁶ is

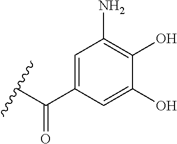 [SMILES: *C(=O)c1cc(N)c(O)c(O)c1]

In embodiments, R²⁶ is

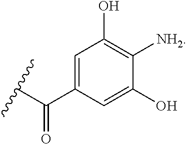 [SMILES: *C(=O)c1cc(O)c(N)c(O)c1]

In embodiments, R²⁶ is

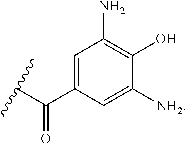 [SMILES: *C(=O)c1cc(N)c(O)c(N)c1]

In embodiments, R²⁶ is

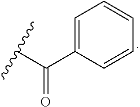 [SMILES: *C(=O)c1ccccc1]

In embodiments, R²⁶ is

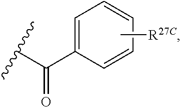

wherein R^{27C} is as described herein. In embodiments, R²⁶ is —C(O)R^{26C} and R^{26C} is R^{27C}-substituted or unsubstituted 5 to 6 membered heteroaryl. In embodiments, R²⁶ is —C(O)R^{26C} and R^{26C} is unsubstituted 5 to 6 membered heteroaryl. In embodiments, R²⁶ is —C(O)R^{26C} and R^{26C} is R^{27C}-substituted or unsubstituted pyridyl. In embodiments, R²⁶ is —C(O)R^{26C} and R^{26C} is R^{27C}-substituted or unsubstituted thienyl. In embodiments, R²⁶ is —C(O)R^{26C} and R^{26C} is R^{27C}-substituted or unsubstituted pyrimidinyl. In embodiments, R²⁶ is —C(O)R^{26C} and R^{26C} is R^{27C}-substituted or unsubstituted pyrazinyl. In embodiments, R²⁶ is —C(O)R^{26C} and R^{26C} is R^{27C}-substituted or unsubstituted pyridazinyl. In embodiments, R²⁶ is —C(O)R^{26C} and R^{26C} is R^{27C}-substituted or unsubstituted triazinyl. In embodiments, R²⁶ is —C(O)R^{26C} and R^{26C} is R^{27C}-substituted or unsubstituted pyrrolyl. In embodiments, R²⁶ is —C(O)R^{26C} and R^{26C} is R^{27C}-substituted or unsubstituted furanyl. In embodiments, R²⁶ is —C(O)R^{26C} and R^{26C} is R^{27C}-substituted or unsubstituted imidazolyl. In embodiments, R²⁶ is —C(O)R^{26C} and R^{26C} is R^{27C}-substituted or unsubstituted pyrazolyl. In embodiments, R²⁶ is —C(O)R^{26C} and R^{26C} is R^{27C}-substituted or unsubstituted oxazolyl. In embodiments, R²⁶ is —C(O)R^{26C} and R^{26C} is R^{27C}-substituted or unsubstituted isoxazolyl. In embodiments, R²⁶ is —C(O)R^{26C} and R^{26C} is R^{27C}-substituted or unsubstituted thiazolyl. In embodiments, R²⁶ is C(O)R^{26C} and R^{26C} is R^{27C}-substituted or unsubstituted isothiazolyl.

In embodiments, the compound is

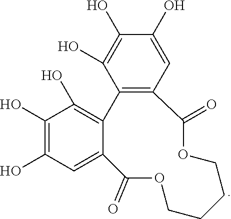 [SMILES: O=C1OCCCCOC(=O)c2cc(O)c(O)c(O)c2-c2c1cc(O)c(O)c2O]

In embodiments, the compound is

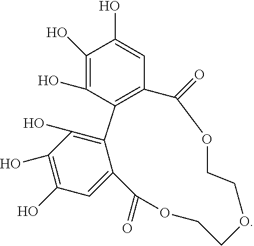 [SMILES: O=C1OCCOCCOC(=O)c2cc(O)c(O)c(O)c2-c2c1cc(O)c(O)c2O]

In embodiments, the compound is

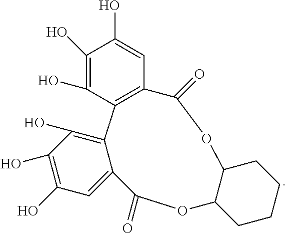 [SMILES: O=C1OC2CCCCC2OC(=O)c2cc(O)c(O)c(O)c2-c2c1cc(O)c(O)c2O]

In embodiments, the compound is

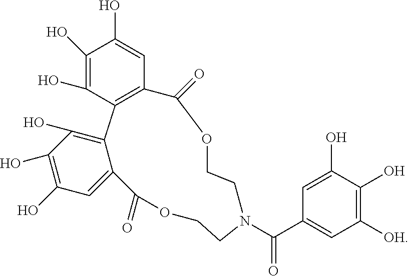 [SMILES: O=C1OCCN(C(=O)c2cc(O)c(O)c(O)c2)CCOC(=O)c2cc(O)c(O)c(O)c2-c2c1cc(O)c(O)c2O]

In embodiments, the compound is

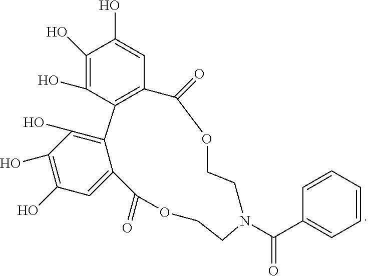 [SMILES: O=C1OCCN(C(=O)c2ccccc2)CCOC(=O)c2cc(O)c(O)c(O)c2-c2c1cc(O)c(O)c2O]

In embodiments, the compound is

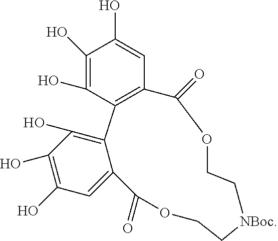 [SMILES: CC(C)(C)OC(=O)N1CCOC(=O)c2cc(O)c(O)c(O)c2-c2c(cc(O)c(O)c2O)C(=O)OCC1]

In embodiments, the compound is

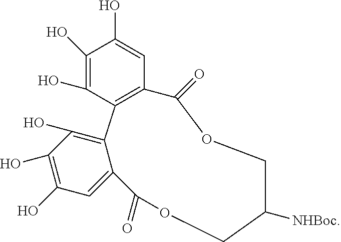 [SMILES: CC(C)(C)OC(=O)NC1COC(=O)c2cc(O)c(O)c(O)c2-c2c(cc(O)c(O)c2O)C(=O)OC1]

In embodiments, the compound is

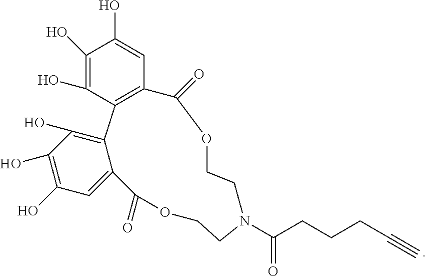 [SMILES: C#CCCCC(=O)N1CCOC(=O)c2cc(O)c(O)c(O)c2-c2c(cc(O)c(O)c2O)C(=O)OCC1]

In embodiments, the compound is

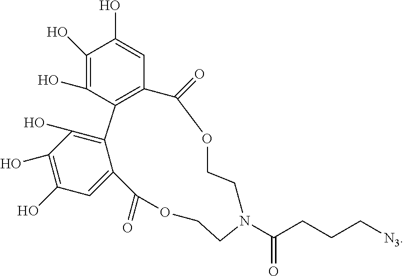 [SMILES: [N-]=[N+]=NCCCC(=O)N1CCOC(=O)c2cc(O)c(O)c(O)c2-c2c(cc(O)c(O)c2O)C(=O)OCC1]

In embodiments, the compound is

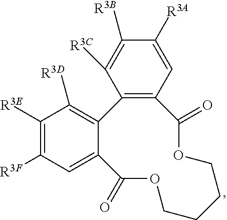

wherein R^{3A}, R^{3B}, R^{3C}, R^{3D}, and R^{3E} are as described herein. In embodiments, the compound is

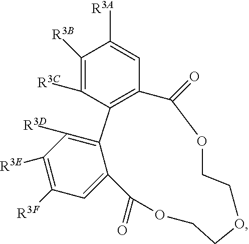

wherein R^{3A}, R^{3B}, R^{3C}, R^{3D}, and R^{3E} are as described herein. In embodiments, the compound is

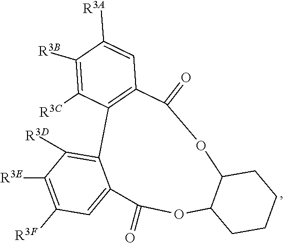

wherein R^{3A}, R^{3B}, R^{3C}, R^{3D}, and R^{3E} are as described herein. In embodiments, the compound is

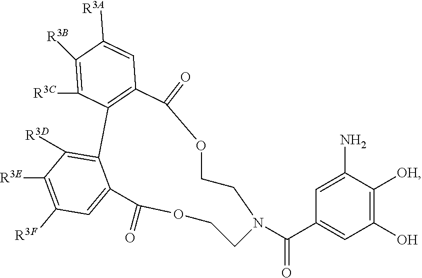

wherein R^{3A}, R^{3B}, R^{3C}, R^{3D}, and R^{3E} are as described herein. In embodiments, the compound is

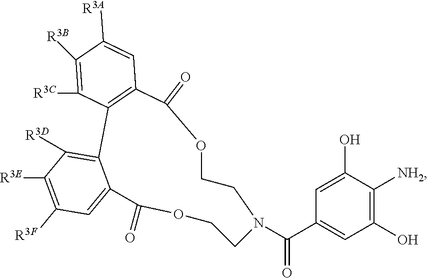

wherein R^{3A}, R^{3B}, R^{3C}, R^{3D}, and R^{3E} are as described herein. In embodiments, the compound is

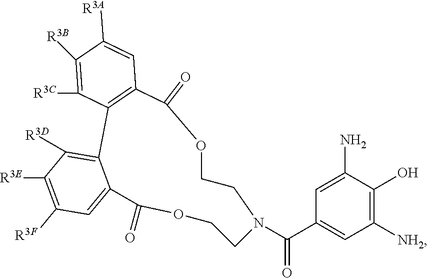

wherein R^{3A}, R^{3B}, R^{3C}, R^{3D}, and R^{3E} are as described herein. In embodiments, the compound is

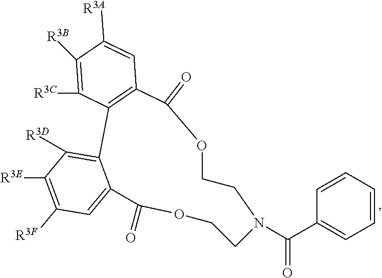

wherein R^{3A}, R^{3B}, R^{3C}, R^{3D}, and R^{3E} are as described herein. In embodiments, the compound

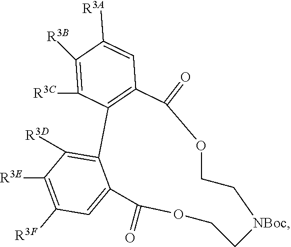

wherein R^{3A}, R^{3B}, R^{3C}, R^{3D}, and R^{3E} are as described herein.

In embodiments, the compound is

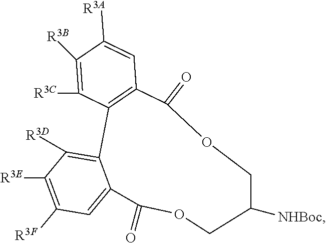

wherein R^{3A}, R^{3B}, R^{3C}, R^{3D}, R^{3E} are as described herein. In embodiments, the compound is

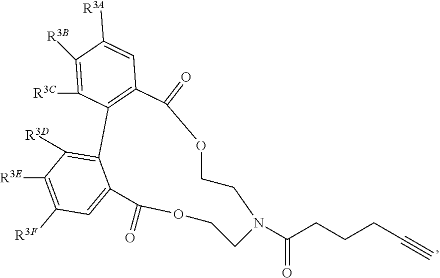

wherein R^{3A}, R^{3B}, R^{3C}, R^{3D}, and R^{3E} are as described herein. In embodiments, the compound is

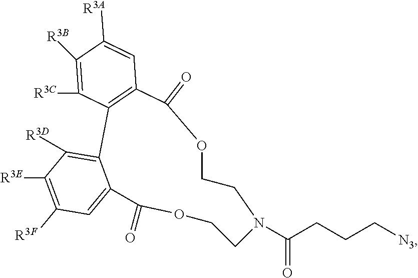

wherein R^{3A}, R^{3B}, R^{3C}, R^{3D}, and R^{3E} are as described herein.

In embodiments, R^{3A}, R^{3B}, R^{3C}, R^{3D}, and R^{3E} are —OH and R^{3F} is —NH₂. In embodiments, R^{3A}, R^{3B}, R^{3C}, R^{3D}, and R^{3F} are —OH and R^{3E} is —NH₂. In embodiments, R^{3A}, R^{3B}, R^{3C}, R^{3F}, and R^{3E} are —OH and R^{3D} is —NH₂. In embodiments, R^{3A}, R^{3B}, R^{3F}, R^{3D}, and R^{3E} are —OH and R^{3C} is —NH₂. In embodiments, R^{3A}, R^{3F}, R^{3C}, R^{3D}, and R^{3E} are —OH and R^{3B} is —NH₂. In embodiments, R^{3F}, R^{3B}, R^{3C}, R^{3D}, and R^{3E} are —OH and R^{3A} is —NH₂. In embodiments, R^{3B}, R^{3D}, R^{3E}, and R^{3F} are —OH; R^{3A} and R^{3C} are —NH₂. In embodiments, R^{3B}, R^{3C}, R^{3E}, and R^{3F} are —OH; R^{3A} and R^{3D} are —NH₂. In embodiments, R^{3B}, R^{3D}, R^{3E}, and R^{3C} are —OH; R^{3A} and R^{3F} are —NH₂. In embodiments, R^{3B}, R^{3A}, R^{3E}, and R^{3F} are —OH; R^{3D} and R^{3C} are —NH₂. In embodiments, R^{3B}, R^{3D}, R^{3E}, and R^{3A} are —OH; R^{3F} and R^{3C} are —NH₂. In embodiments, R^{3B}, R^{3A}, R^{3E}, and R^{3C} are —OH; R^{3D} and R^{3F} are —NH₂. In embodiments, R^{3A}, R^{3D}, R^{3C}, and R^{3F} are —OH; R^{3B} and R^{3E} are —NH₂. In embodiments, five of R^{3A}, R^{3B}, R^{3C}, R^{3D}, R^{3E}, and R^{3F} are —OH and one of R^{3A}, R^{3B}, R^{3C}, R^{3D}, R^{3E}, and R^{3F} is —NH₂. In embodiments, four of R^{3A}, R^{3B}, R^{3C}, R^{3D}, R^{3E}, and R^{3F} are —OH and two of R^{3A}, R^{3B}, R^{3C}, R^{3D}, R^{3E}, and R^{3F} are —NH₂. In embodiments, three of R^{3A}, R^{3B}, R^{3C}, R^{3D}, R^{3E}, and R^{3F} are —OH and three of R^{3A}, R^{3B}, R^{3C}, R^{3D}, R^{3E}, and R^{3F} are —NH₂. In embodiments, two of R^{3A}, R^{3B}, R^{3C}, R^{3D}, R^{3E}, and R^{3F} are —OH and four of R^{3A}, R^{3B}, R^{3C}, R^{3D}, R^{3E}, and R^{3F} are —NH₂. In embodiments, one of R^{3A}, R^{3B}, R^{3C}, R^{3D}, R^{3E}, and R^{3F} is OH and five of R^{3A}, R^{3B}, R^{3C}, R^{3D}, R^{3E}, and R^{3F} are —NH₂.

In embodiments, L¹ includes a bioconjugate reactive group (e.g., alkynyl, —N₃). In embodiments, R²⁶ includes a bioconjugate reactive group (e.g., alkynyl, —N₃). In embodiments R^{27A}, R^{27B}, R^{27C}, R^{27D}, R^{28A}, R^{28B}, R^{28C}, R^{28D}, R^{29A}, R^{29B}, R^{29C}, R^{29D}, R^{27AA}, R^{27AB}, R^{27AC}, R^{27AD}, R^{27BA}, R^{27BB}, R^{27BC}, R^{27BD}, R^{27CA}, R^{27CB}, R^{27CC}, R^{27CD}, R^{27DA}, R^{27DB}, R^{27DC}, and R^{27DD} independently comprise a bioconjugate reactive group (e.g., alkynyl, —N₃). In embodiments, R²⁶ is a bioconjugate reactive group (e.g., alkynyl, —N₃). In embodiments R^{27A}, R^{27B}, R^{27C}, R^{27D}, R^{28A}, R^{28B}, R^{28C}, R^{28D}, R^{29A}, R^{29B}, R^{29C}, R^{29D}, R^{27AA}, R^{27AB}, R^{27AC}, R^{27AD}, R^{27BA}, R^{27BB}, R^{27BC}, R^{27BD}, R^{27CA}, R^{27CB}, R^{27CC}, R^{27CD}, R^{27DA}, R^{27DB}, R^{27DC}, and R^{27DD} independently are a bioconjugate reactive group (e.g., alkynyl, —N₃).

In embodiments, a linker (e.g., bioconjugate linker) is formed by a conjugation or bioconjugation reaction combining a first reactant (e.g., bioconjugate reactant) moiety covalently bonded to L¹ and a second reactant (e.g., bioconjugate reactant) moiety. In embodiments, a linker (e.g., bioconjugate linker) is formed by a conjugation or bioconjugation reaction combining a first reactant (e.g., bioconjugate reactant) moiety covalently bonded to R²⁶ and a second reactant (e.g., bioconjugate reactant) moiety. In embodiments, a linker (e.g., bioconjugate linker) is formed by a conjugation or bioconjugation reaction combining a first reactant (e.g., bioconjugate reactant) moiety covalently bonded to R^{27A}, R^{27B}, R^{27C}, R^{27D}, R^{28A}, R^{28B}, R^{28C}, R^{28D}, R^{29A}, R^{29B}, R^{29C}, R^{29D}, R^{27AA}, R^{27AB}, R^{27AC}, R^{27AD}, R^{27BA}, R^{27BB}, R^{27BC}, R^{27BD}, R^{27CA}, R^{27CB}, R^{27CC}, R^{27CD}, R^{27DA}, R^{27DB}, R^{27DC}, or R^{27DD} and a second reactant (e.g., bioconjugate reactant) moiety.

In some embodiments, a compound as described herein may include multiple instances of R²⁶ and/or other variables. In such embodiments, each variable may optional be different and be appropriately labeled to distinguish each group for greater clarity. For example, where each R²⁶ is different, they may be referred to, for example, as R^{26.1}, R^{26.2}, R^{26.3}, R^{26.4}, R^{26.5}, respectively, wherein the definition of R²⁶ is assumed by R^{26.1}, R^{26.2}, R^{26.3}, R^{26.4}, R^{26.5}. The variables used within a definition of R²⁶ and/or other variables that appear at multiple instances and are different may similarly be appropriately labeled to distinguish each group for greater clarity. In some embodiments, the compound is a compound described herein (e.g., in an aspect, embodiment, example, claim, table, scheme, drawing, or figure).

In embodiments, unless otherwise indicated, a compound described herein is a racemic mixture of all stereoisomers. In embodiments, unless otherwise indicated, a compound described herein is a racemic mixture of all enantiomers. In embodiments, unless otherwise indicated, a compound described herein is a racemic mixture of two opposite stereoisomers. In embodiments, unless otherwise indicated, a compound described herein is a racemic mixture of two opposite enantiomers. In embodiments, unless otherwise indicated, a compound described herein is a single stereoisomer. In embodiments, unless otherwise indicated, a compound described herein is a single enantiomer. In embodiments, the compound is a compound described herein (e.g., in an aspect, embodiment, example, figure, table, scheme, or claim).

### III. Pharmaceutical Compositions

In an aspect is provided a pharmaceutical composition including a compound described herein, or pharmaceutically acceptable salt thereof, and a pharmaceutically acceptable excipient.

In embodiments of the pharmaceutical compositions, the compound, or pharmaceutically acceptable salt thereof, is included in a therapeutically effective amount.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating cancer. In embodiments, the second agent is an anti-cancer agent. In embodiments, the second agent is a chemotherapeutic. In embodiments, the second agent is an anti-fibrotic agent.

### IV. Methods of Treatment

In an aspect is provided a method of treating cancer including administering to a subject in need thereof an effective amount of a compound described herein. In embodiments, the cancer is breast cancer. In embodiments, the cancer is pancreatic cancer. In embodiments, the cancer is liver cancer. In embodiments, the cancer is metastatic cancer. In embodiments, the cancer is lung cancer. In embodiments, the cancer is non-small cell lung cancer. In embodiments, the cancer is metastatic lung cancer.

In an aspect is provided a method of treating a fibrotic pulmonary disease including administering to a subject in need thereof an effective amount of a compound described herein.

In an aspect is provided a method of treating acute lung injury including administering to a subject in need thereof an effective amount of a compound described herein.

In an aspect is provided a method of treating a pulmonary fibrotic condition including administering to a subject in need thereof an effective amount of a compound described herein.

In an aspect is provided a method of treating a pulmonary disease including administering to a subject in need thereof an effective amount of a compound described herein.

In an aspect is provided a method of treating a fibrotic disease (e.g., fibrosis, cirrhosis) including administering to a subject in need thereof an effective amount of a compound described herein. In embodiments, the method includes treating cirrhosis.

In an aspect is provided a method of treating fibrosis including administering to a subject in need thereof an effective amount of a compound described herein. In embodiments, the disease is pulmonary fibrosis. In embodiments, the disease is idiopathic pulmonary fibrosis. In embodiments, the method includes reducing the level of collagen crosslinking. In embodiments, the method includes reducing the level of collagen. In embodiments, the method includes reducing the level of elastin crosslinking. In embodiments, the method includes reducing the level of elastin.

In an aspect is provided a method of treating a disease associated with Lysyl oxidase homolog 2 (LOXL2) protein activity including administering to a subject in need thereof an effective amount of a compound described herein. In embodiments, the method includes reducing the level of collagen crosslinking. In embodiments, the method includes reducing the level of snail 1 protein. In embodiments, the method includes reducing the level of snail 1 activity. In embodiments, the method includes reducing the level of elastin crosslinking.

In an aspect is provided a method of treating a disease associated with TGF-β1 protein activity including administering to a subject in need thereof an effective amount of a compound described herein. In embodiments, the method includes reducing the level of collagen crosslinking. In embodiments, the method includes reducing the level of TGF-β1 protein. In embodiments, the method includes reducing the level of TGF-β1 activity. In embodiments, the method includes reducing the level of elastin crosslinking.

In an aspect is provided a method of treating a disease associated with TGFβRI protein activity including administering to a subject in need thereof an effective amount of a compound described herein. In embodiments, the method includes reducing the level of collagen crosslinking. In embodiments, the method includes reducing the level of TGFβRI protein. In embodiments, the method includes reducing the level of TGFβRI activity. In embodiments, the method includes reducing the level of elastin crosslinking.

In an aspect is provided a method of treating a disease associated with SNAIL1 protein activity including administering to a subject in need thereof an effective amount of a compound described herein.

In an aspect is provided a method of treating fibrosis, the method including administering to a subject in need thereof an effective amount of a compound described herein. In an aspect is provided a method of treating pulmonary fibrosis, the method including administering to a subject in need thereof an effective amount of a compound described herein. In an aspect is provided a method of treating idiopathic pulmonary fibrosis, the method including administering to a subject in need thereof an effective amount of a compound described herein. In an aspect is provided a method of treating cancer, the method including administering to a subject in need thereof an effective amount of a compound described herein. In an aspect is provided a method of treating cancer metastasis, the method including administering to a subject in need thereof an effective amount of a compound described herein. In embodiments, the method includes reducing the level of collagen (e.g., lung collagen, tumor collagen, collagen near a tumor, collagen associated with a tumor). In embodiments, the method includes reducing the level of elastin (e.g., lung elastin, tumor elastin, elastin near a tumor, elastin associated with a tumor).

In an aspect is provided a method of treating cancer including administering to a subject in need thereof an effective amount of a TGFβRI inhibitor, TGFβRI inhibitor compound, LOXL2 generated TGFβRI inhibitor (e.g., LOXL2 generated TGFβRI inhibitor compound), LOXL2 generated TGFβRI inhibitor precursor (e.g., LOXL2 generated TGFβRI inhibitor compound precursor), or compound described herein. In embodiments, the cancer is breast cancer. In embodiments, the cancer is pancreatic cancer. In embodiments, the cancer is liver cancer. In embodiments, the cancer is metastatic cancer. In embodiments, the cancer is lung cancer. In embodiments, the cancer is non-small cell lung cancer. In embodiments, the cancer is metastatic lung cancer.

In an aspect is provided a method of treating a fibrotic pulmonary disease including administering to a subject in need thereof an effective amount of a TGFβRI inhibitor, TGFβRI inhibitor compound, LOXL2 generated TGFβRI inhibitor (e.g., LOXL2 generated TGFβRI inhibitor compound), LOXL2 generated TGFβRI inhibitor precursor (e.g., LOXL2 generated TGFβRI inhibitor compound precursor), or compound described herein.

In an aspect is provided a method of treating acute lung injury including administering to a subject in need thereof an effective amount of a TGFβRI inhibitor, TGFβRI inhibitor compound, LOXL2 generated TGFβRI inhibitor (e.g., LOXL2 generated TGFβRI inhibitor compound), LOXL2 generated TGFβRI inhibitor precursor (e.g., LOXL2 generated TGFβRI inhibitor compound precursor), or compound described herein.

In an aspect is provided a method of treating a pulmonary fibrotic condition including administering to a subject in need thereof an effective amount of a TGFβRI inhibitor, TGFβRI inhibitor compound, LOXL2 generated TGFβRI inhibitor (e.g., LOXL2 generated TGFβRI inhibitor compound), LOXL2 generated TGFβRI inhibitor precursor (e.g., LOXL2 generated TGFβRI inhibitor compound precursor), or compound described herein.

In an aspect is provided a method of treating a pulmonary disease including administering to a subject in need thereof an effective amount of a TGFβRI inhibitor, TGFβRI inhibitor compound, LOXL2 generated TGFβRI inhibitor (e.g., LOXL2 generated TGFβRI inhibitor compound), LOXL2 generated TGFβRI inhibitor precursor (e.g., LOXL2 generated TGFβRI inhibitor compound precursor), or compound described herein.

In an aspect is provided a method of treating fibrosis including administering to a subject in need thereof an effective amount of a TGFβRI inhibitor, TGFβRI inhibitor compound, LOXL2 generated TGFβRI inhibitor (e.g., LOXL2 generated TGFβRI inhibitor compound), LOXL2 generated TGFβRI inhibitor precursor (e.g., LOXL2 generated TGFβRI inhibitor compound precursor), or compound described herein. In embodiments, the disease is pulmonary fibrosis. In embodiments, the disease is idiopathic pulmonary fibrosis. In embodiments, the method includes reducing the level of collagen crosslinking. In embodiments, the method includes reducing the level of collagen. In embodiments, the method includes reducing the level of elastin crosslinking. In embodiments, the method includes reducing the level of elastin.

In embodiments, the method includes administering a second agent (e.g. therapeutic agent). In embodiments, the method includes administering a second agent (e.g. therapeutic agent) in a therapeutically effective amount. In embodiments, the second agent is an agent for treating cancer. In embodiments, the second agent is an anti-cancer agent. In embodiments, the second agent is a chemotherapeutic. In embodiments, the second agent is an anti-fibrosis agent.

In embodiments, the method does not include reducing bone density in the subject compared to absence of the compound. In embodiments, the method does not include reducing systemic collagen (e.g., aortic collagen) compared to absence of the compound. In embodiments, the method of treatment includes reducing the level of LOXL2 protein (e.g., as described herein below, compared to control, compared to absence of the compound). In embodiments, the method of treatment includes reducing the level of LOXL2 activity (e.g., as described herein below, compared to control, compared to absence of the compound). In embodiments, the method of treatment includes reducing the level of TGF-β1 protein (e.g., as described herein below, compared to control, compared to absence of the compound). In embodiments, the method of treatment includes reducing the level of TGF-β1 activity (e.g., as described herein below, compared to control, compared to absence of the compound). In embodiments, the method of treatment includes reducing the level of Transforming growth factor beta 1 (TGF-β1) signal transduction pathway activity (e.g., as described herein below, compared to control, compared to absence of the compound). In embodiments, the method of treatment includes reducing the level of snail 1 protein (e.g., as described herein below, compared to control, compared to absence of the compound). In embodiments, the method of treatment includes reducing the level of snail 1 activity (e.g., as described herein below, compared to control, compared to absence of the compound). In embodiments, the method of treatment includes reducing the level of TGFβRI protein (e.g., as described herein below, compared to control, compared to absence of the compound). In embodiments, the method of treatment includes reducing the level of TGFβRI activity (e.g., as described herein below, compared to control, compared to absence of the compound). In embodiments, the method of treatment includes reducing the level of Transforming growth factor beta Receptor 1 (TGFβRI) signal transduction pathway activity (e.g., as described herein below, compared to control, compared to absence of the compound).

In embodiments, the method includes inhibiting (e.g., reducing compared to control, reducing compared to absence of the compound) LOXL2 protein activity and Transforming growth factor beta Receptor 1 (TGFβRI) protein activity in a cell, including contacting the LOXL2 protein with a LOXL2 generated TGFβRI inhibitor precursor (e.g., LOXL2 generated TGFβRI inhibitor compound precursor), wherein the LOXL2 protein modifies the LOXL2 generated TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound); and wherein the LOXL2 generated TGFβRI inhibitor (e.g., LOXL2 generated TGFβRI inhibitor compound) contacts a TGFβRI protein. In embodiments, the LOXL2 protein is inhibited by the LOXL2 generated TGFβRI inhibitor precursor (e.g., LOXL2 generated TGFβRI inhibitor compound precursor). In embodiments, the LOXL2 protein is inhibited by the LOXL2 reaction converting the LOXL2 generated TGFβRI inhibitor precursor (e.g., LOXL2 generated TGFβRI inhibitor compound precursor) to a TGFβRI inhibitor (e.g., LOXL2 generated TGFβRI inhibitor compound). In embodiments, the inhibition is in a cell expressing LOXL2 and not outside of the cell.

### V. Methods of Inhibition

In an aspect is provided a method of inhibiting (e.g., reducing compared to control, reducing compared to absence of the compound) Lysyl oxidase homolog 2 (LOXL2) protein activity including contacting the LOXL2 protein with a compound described herein. In embodiments, the LOXL2 protein is a human LOXL2 protein. In embodiments, the method includes reducing the level of collagen crosslinking (e.g., reducing compared to control, reducing compared to absence of the compound). In embodiments, the method includes reducing the level of snail 1 protein (e.g., reducing compared to control, reducing compared to absence of the compound). In embodiments, the method includes reducing the level of snail 1 activity (e.g., reducing compared to control, reducing compared to absence of the compound). In embodiments, the LOXL2 is intracellular. In embodiments, the LOXL2 is extracellular. In embodiments, the inhibition is in a cell expressing LOXL2. In embodiments, the inhibition is in a cell expressing LOXL2 and not outside of the cell. In embodiments, the method includes reducing the level of elastin crosslinking (e.g., reducing compared to control, reducing compared to absence of the compound). In embodiments, the method includes modulating the amino acid in LOXL2 corresponding to K731 in human LOXL2. In embodiments, the method includes changing the amino acid corresponding to K731 in human LOXL2 to an allysine residue (e.g., converting the terminal sidechain —CH₂NH₂ of the amino acid residue to —CH(O)).

In an aspect is provided a method of inhibiting (e.g., reducing compared to control, reducing compared to absence of the compound) Lysyl oxidase homolog 2 (LOXL2) protein activity including contacting the LOXL2 protein with a LOXL2 generated TGFβRI inhibitor precursor (e.g., LOXL2 generated TGFβRI inhibitor compound precursor, compound described herein). In embodiments, the LOXL2 protein is a human LOXL2 protein. In embodiments, the method includes reducing the level of collagen crosslinking (e.g., reducing compared to control, reducing compared to absence of the compound). In embodiments, the method includes reducing the level of snail 1 protein (e.g., reducing compared to control, reducing compared to absence of the compound). In embodiments, the method includes reducing the level of snail 1 activity (e.g., reducing compared to control, reducing compared to absence of the compound). In embodiments, the LOXL2 is intracellular. In embodiments, the LOXL2 is extracellular. In embodiments, the inhibition is in a cell expressing LOXL2. In embodiments, the inhibition is in a cell expressing LOXL2 and not outside of the cell. In embodiments, the method includes reducing the level of elastin crosslinking (e.g., reducing compared to control, reducing compared to absence of the compound). In embodiments, the method includes modulating the amino acid in LOXL2 corresponding to K731 in human LOXL2. In embodiments, the method includes changing the amino acid corresponding to K731 in human LOXL2 to an allysine residue (e.g., converting the terminal sidechain —CH₂NH₂ of the amino acid residue to —CH(O)). In embodiments, LOXL2 modifies the LOXL2 generated TGFβRI inhibitor precursor (e.g., LOXL2 generated TGFβRI inhibitor compound precursor, compound described herein) to make a LOXL2 generated TGFβRI inhibitor (e.g., LOXL2 generated TGFβRI inhibitor compound, compound described herein). In embodiments, the method includes inhibition of TGFβRI with the LOXL2 generated TGFβRI inhibitor (e.g., LOXL2 generated TGFβRI inhibitor compound, compound described herein) made by LOXL2 from the LOXL2 generated TGFβRI inhibitor precursor (e.g., LOXL2 generated TGFβRI inhibitor compound precursor, compound described herein).

In an aspect is provided a method of inhibiting (e.g., reducing compared to control, reducing compared to absence of the compound) SNAIL1 protein activity including contacting the SNAIL1 protein with a compound described herein. In embodiments, the SNAIL1 protein is a human SNAIL1 protein. In embodiments, the inhibition is in a cell expressing LOXL2. In embodiments, the level of inhibition is proportional to the level of LOXL2 protein in a cell. In embodiments, the level of inhibition is proportional to the level of LOXL2 activity in a cell. In embodiments, the inhibition of SNAIL1 protein is in a cell expressing LOXL2 and not outside of the cell. In embodiments, the inhibition of SNAIL1 accumulation is in a cell expressing LOXL2 and not outside of the cell. In embodiments, the inhibition of SNAIL1 activity is in a cell expressing LOXL2 and not outside of the cell.

In an aspect is provided a method of reducing the level of activity of zinc finger protein Snail1 in a subject (e.g., reducing compared to control, reducing compared to absence of the compound), the method including administering an effective amount of a compound described herein to the subject. In an aspect is provided a method of reducing the level of activity of Lysyl oxidase homolog 2 in a subject (e.g., reducing compared to control, reducing compared to absence of the compound), the method including administering an effective amount of a compound described herein to the subject. In an aspect is provided a method of inhibiting collagen cross-linking in a subject (e.g., reducing compared to control, reducing compared to absence of the compound), the method including administering an effective amount of a compound described herein to the subject. In an aspect is provided a method of inhibiting elastin cross-linking in a subject (e.g., reducing compared to control, reducing compared to absence of the compound), the method including administering an effective amount of a compound described herein to the subject. In embodiments, the inhibition is in a cell expressing LOXL2 and not outside of the cell.

In an aspect is provided a method of inhibiting (e.g., reducing compared to control, reducing compared to absence of the compound) Transforming growth factor beta 1 (TGF-β1) protein activity including contacting the TGF-β1 protein with a compound described herein. In embodiments, the TGF-β1 protein is a human TGF-β1 protein. In embodiments, the inhibition is in a cell expressing LOXL2 and not outside of the cell. In embodiments the compound inhibits LOXL2 activity and TGF-β1 activity in the same cell (e.g., reducing compared to control, reducing compared to absence of the compound, in a fibroblast cell).

In an aspect is provided a method of inhibiting (e.g., reducing compared to control, reducing compared to absence of the compound) Transforming growth factor beta 1 (TGF-β1) signal transduction pathway activity of a cell including contacting the cell with a compound described herein. In embodiments, the TGF-β1 is a human TGF-β1. In embodiments, the cell is a fibroblast. In embodiments, the method includes reducing the level of fibronectin activity (e.g., reducing compared to control, reducing compared to absence of the compound). In embodiments, the method includes reducing the level of fibronectin protein (e.g., reducing compared to control, reducing compared to absence of the compound). In embodiments, the method includes increasing the level of E-cadherin protein (e.g., increasing compared to control, increasing compared to absence of the compound). In embodiments, the method includes increasing the level of E-cadherin activity (e.g., increasing compared to control, increasing compared to absence of the compound). In embodiments, the method includes reducing the level of collagen protein (e.g., reducing compared to control, reducing compared to absence of the compound). In embodiments, the method includes reducing the level of Snail1 activity (e.g., reducing compared to control, reducing compared to absence of the compound). In embodiments, the method includes reducing the level of Snail1 protein (e.g., reducing compared to control, reducing compared to absence of the compound). In embodiments, the method includes reducing the level of p-smad3 activity (e.g., reducing compared to control, reducing compared to absence of the compound). In embodiments, the method includes reducing the level of p-smad3 protein (e.g., reducing compared to control, reducing compared to absence of the compound). In embodiments, the method includes reducing the level of one or more epithelial-mesenchymal transition (EMT) associated markers (e.g., proteins) (e.g., reducing compared to control, reducing compared to absence of the compound). In embodiments, the method includes reducing the level of collagen crosslinking (e.g., reducing compared to control, reducing compared to absence of the compound). In embodiments, the inhibition of TGF-β1 is in a cell expressing LOXL2 and not outside of the cell. In embodiments, the method includes reducing the level of elastin crosslinking (e.g., reducing compared to control, reducing compared to absence of the compound). In embodiments, the method includes reducing the level of elastin protein (e.g., reducing compared to control, reducing compared to absence of the compound). In embodiments the compound inhibits LOXL2 activity and TGF-β1 signal transduction pathway activity in the same cell (e.g., reducing compared to control, reducing compared to absence of the compound, in a fibroblast cell). In embodiments the TGF-31 signal transduction pathway activity is snail 1 activity. In embodiments the TGF-β1 signal transduction pathway activity is Akt activity. In embodiments the TGF-β1 signal transduction pathway activity is PI3K activity.

In an aspect is provided a method of inhibiting (e.g., reducing compared to control, reducing compared to absence of the compound) Transforming growth factor beta Receptor 1 (TGFβRI) protein activity including contacting the TGFβRI protein with a compound described herein. In embodiments, the TGFβRI protein is a human TGFβRI protein. In embodiments, the inhibition is in a cell expressing LOXL2 and not outside of the cell. In embodiments the compound inhibits LOXL2 activity and TGFβRI activity in the same cell (e.g., reducing compared to control, reducing compared to absence of the compound, in a fibroblast cell).

In an aspect is provided a method of inhibiting (e.g., reducing compared to control, reducing compared to absence of the compound) Transforming growth factor beta Receptor 1 (TGFβRI) protein activity including contacting the TGFβRI protein with a TGFβRI inhibitor (e.g., TGFβRI inhibitor compound, compound described herein). In embodiments, the TGFβRI protein is a human TGFβRI protein. In embodiments, the inhibition is in a cell expressing LOXL2 and not outside of the cell. In embodiments the method includes inhibiting LOXL2 activity and TGFβRI activity in the same cell (e.g., reducing compared to control, reducing compared to absence of the compound, in a fibroblast cell) (e.g., a LOXL2 generated TGFβRI inhibitor precursor contacts LOXL2 protein; LOXL2 protein converts TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound) and LOXL2 protein activity is inhibited due to the conversion; the LOXL2 generated TGFβRI inhibitor then contacts TGFβRI protein and inhibits TGFβRI protein). In embodiments, the TGFβRI inhibitor is not a protein or peptide. In embodiments, the TGFβRI inhibitor is a TGFβRI inhibitor compound (e.g., a small molecule, less than 1 kDa, less than 500 Da, less than 250 Da). In embodiments, the TGFβRI inhibitor is a LOXL2 generated transforming growth factor beta Receptor 1 inhibitor (e.g., LOXL2 generated TGFβRI inhibitor compound). In embodiments, the TGFβRI inhibitor is a LOXL2 generated TGFβRI inhibitor precursor (e.g., LOXL2 generated TGFβRI inhibitor compound precursor). In embodiments, the LOXL2 generated TGFβRI inhibitor precursor (e.g., LOXL2 generated TGFβRI inhibitor compound precursor) includes a trihydroxyphenol moiety. In embodiments, the LOXL2 generated TGFβRI inhibitor (e.g., LOXL2 generated TGFβRI inhibitor compound) includes a 1-amino-2,3-dihydroxyphenol moiety.

In an aspect is provided a method of inhibiting (e.g., reducing compared to control, reducing compared to absence of the compound) Transforming growth factor beta Receptor 1 (TGFβRI) protein activity including contacting the TGFβRI protein with a compound described herein. In embodiments, the TGFβRI protein is a human TGFβRI protein. In embodiments, the inhibition is in a cell expressing LOXL2 and not outside of the cell. In embodiments the compound inhibits LOXL2 activity and TGFβRI activity in the same cell (e.g., reducing compared to control, reducing compared to absence of the compound, in a fibroblast cell).

In an aspect is provided a method of inhibiting (e.g., reducing compared to control, reducing compared to absence of the compound) Transforming growth factor beta Receptor 1 (TGFβRI) signal transduction pathway activity of a cell including contacting the cell with a TGFβRI inhibitor (e.g., TGFβRI inhibitor compound, compound described herein). In embodiments, the TGFβRI inhibitor is a TGFβRI inhibitor compound (e.g., a small molecule, less than 1 kDa, less than 500 Da, less than 250 Da). In embodiments, the TGFβRI is a human TGFβRI.

In an aspect is provided a method of inhibiting (e.g., reducing compared to control, reducing compared to absence of the compound) Transforming growth factor beta Receptor 1 (TGFβRI) signal transduction pathway activity of a cell including contacting the cell with a LOXL2 generated TGFβRI inhibitor precursor (e.g., LOXL2 generated TGFβRI inhibitor compound precursor). In embodiments, the LOXL2 generated TGFβRI inhibitor precursor is a LOXL2 generated TGFβRI inhibitor compound precursor (e.g., a small molecule, less than 1 kDa, less than 500 Da, less than 250 Da). In embodiments, the TGFβRI is a human TGFβRI.

In embodiments, the cell is a fibroblast. In embodiments, the method includes reducing the level of fibronectin activity (e.g., reducing compared to control, reducing compared to absence of the compound). In embodiments, the method includes reducing the level of fibronectin protein (e.g., reducing compared to control, reducing compared to absence of the compound). In embodiments, the method includes increasing the level of E-cadherin protein (e.g., increasing compared to control, increasing compared to absence of the compound). In embodiments, the method includes increasing the level of E-cadherin activity (e.g., increasing compared to control, increasing compared to absence of the compound). In embodiments, the method includes reducing the level of collagen protein (e.g., reducing compared to control, reducing compared to absence of the compound). In embodiments, the method includes reducing the level of Snail1 activity (e.g., reducing compared to control, reducing compared to absence of the compound). In embodiments, the method includes reducing the level of Snail1 protein (e.g., reducing compared to control, reducing compared to absence of the compound). In embodiments, the method includes reducing the level of p-smad3 activity (e.g., reducing compared to control, reducing compared to absence of the compound). In embodiments, the method includes reducing the level of p-smad3 protein (e.g., reducing compared to control, reducing compared to absence of the compound). In embodiments, the method includes reducing the level of one or more epithelial-mesenchymal transition (EMT) associated markers (e.g., proteins) (e.g., reducing compared to control, reducing compared to absence of the compound). In embodiments, the method includes reducing the level of collagen crosslinking (e.g., reducing compared to control, reducing compared to absence of the compound). In embodiments, the inhibition of TGFβRI is in a cell expressing LOXL2 and not outside of the cell. In embodiments, the method includes reducing the level of elastin crosslinking (e.g., reducing compared to control, reducing compared to absence of the compound). In embodiments, the method includes reducing the level of elastin protein (e.g., reducing compared to control, reducing compared to absence of the compound). In embodiments the method includes inhibition of LOXL2 activity and TGFβRI signal transduction pathway activity in the same cell (e.g., reducing compared to control, reducing compared to absence of the compound, in a fibroblast cell) (e.g., reducing compared to control, reducing compared to absence of the compound, in a fibroblast cell) (e.g., the TGFβRI inhibitor is a LOXL2 generated TGFβRI inhibitor made by LOXL2 from a LOXL2 generated TGFβRI inhibitor precursor; a LOXL2 generated TGFβRI inhibitor precursor (e.g., LOXL2 generated TGFβRI inhibitor compound precursor) contacts LOXL2 protein; LOXL2 protein converts TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound) and LOXL2 protein activity is inhibited due to the conversion; the LOXL2 generated TGFβRI inhibitor then contacts TGFβRI protein and inhibits TGFβRI protein). In embodiments the TGFβRI signal transduction pathway activity is snail 1 activity. In embodiments the TGFβRI signal transduction pathway activity is Akt activity. In embodiments the TGFβRI signal transduction pathway activity is PI3K activity. In embodiments the TGFβRI signal transduction pathway activity is the TGFβRI kinase activity. In embodiments, the TGFβRI inhibitor is not a protein or peptide. In embodiments, the TGFβRI inhibitor is a LOXL2 generated transforming growth factor beta Receptor 1 inhibitor (e.g., LOXL2 generated TGFβRI inhibitor compound). In embodiments, the TGFβRI inhibitor is a LOXL2 generated TGFβRI inhibitor precursor (e.g., LOXL2 generated TGFβRI inhibitor compound precursor). In embodiments, the LOXL2 generated TGFβRI inhibitor precursor (e.g., LOXL2 generated TGFβRI inhibitor compound precursor) includes a trihydroxyphenol moiety. In embodiments, the LOXL2 generated TGFβRI inhibitor (e.g., LOXL2 generated TGFβRI inhibitor compound) includes a 1-amino-2,3-dihydroxyphenol moiety.

In an aspect is provided a method of inhibiting (e.g., reducing compared to control, reducing compared to absence of the compound) Transforming growth factor beta Receptor 1 (TGFβRI) signal transduction pathway activity of a cell including contacting the cell with a compound described herein. In embodiments, the TGFβRI is a human TGFβRI. In embodiments, the cell is a fibroblast. In embodiments, the method includes reducing the level of fibronectin activity (e.g., reducing compared to control, reducing compared to absence of the compound). In embodiments, the method includes reducing the level of fibronectin protein (e.g., reducing compared to control, reducing compared to absence of the compound). In embodiments, the method includes increasing the level of E-cadherin protein (e.g., increasing compared to control, increasing compared to absence of the compound). In embodiments, the method includes increasing the level of E-cadherin activity (e.g., increasing compared to control, increasing compared to absence of the compound). In embodiments, the method includes reducing the level of collagen protein (e.g., reducing compared to control, reducing compared to absence of the compound). In embodiments, the method includes reducing the level of Snail1 activity (e.g., reducing compared to control, reducing compared to absence of the compound). In embodiments, the method includes reducing the level of Snail1 protein (e.g., reducing compared to control, reducing compared to absence of the compound). In embodiments, the method includes reducing the level of p-smad3 activity (e.g., reducing compared to control, reducing compared to absence of the compound). In embodiments, the method includes reducing the level of p-smad3 protein (e.g., reducing compared to control, reducing compared to absence of the compound). In embodiments, the method includes reducing the level of one or more epithelial-mesenchymal transition (EMT) associated markers (e.g., proteins) (e.g., reducing compared to control, reducing compared to absence of the compound). In embodiments, the method includes reducing the level of collagen crosslinking (e.g., reducing compared to control, reducing compared to absence of the compound). In embodiments, the inhibition of TGFβRI is in a cell expressing LOXL2 and not outside of the cell. In embodiments, the method includes reducing the level of elastin crosslinking (e.g., reducing compared to control, reducing compared to absence of the compound). In embodiments, the method includes reducing the level of elastin protein (e.g., reducing compared to control, reducing compared to absence of the compound). In embodiments the compound inhibits LOXL2 activity and TGFβRI signal transduction pathway activity in the same cell (e.g., reducing compared to control, reducing compared to absence of the compound, in a fibroblast cell). In embodiments the TGFβRI signal transduction pathway activity is snail 1 activity. In embodiments the TGFβRI signal transduction pathway activity is Akt activity. In embodiments the TGFβRI signal transduction pathway activity is PI3K activity. In embodiments the TGFβRI signal transduction pathway activity is the TGFβRI kinase activity.

In an aspect is provided a method of inhibiting (e.g., reducing compared to control, reducing compared to absence of the compound) LOXL2 protein activity and Transforming growth factor beta Receptor 1 (TGFβRI) protein activity in a cell, including contacting the LOXL2 protein with a LOXL2 generated TGFβRI inhibitor precursor (e.g., LOXL2 generated TGFβRI inhibitor compound precursor), wherein the LOXL2 protein modifies the LOXL2 generated TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound); and wherein the LOXL2 generated TGFβRI inhibitor (e.g., LOXL2 generated TGFβRI inhibitor compound) contacts a TGFβRI protein. In embodiments, the LOXL2 protein is inhibited by the LOXL2 generated TGFβRI inhibitor precursor (e.g., LOXL2 generated TGFβRI inhibitor compound precursor). In embodiments, the LOXL2 protein is inhibited by the LOXL2 reaction converting the LOXL2 generated TGFβRI inhibitor precursor (e.g., LOXL2 generated TGFβRI inhibitor compound precursor) to a TGFβRI inhibitor (e.g., LOXL2 generated TGFβRI inhibitor compound). In embodiments, the inhibition is in a cell expressing LOXL2 and not outside of the cell.

In an aspect is provided a method of inhibiting Lysyl oxidase homolog 2 (LOXL2) protein activity and Transforming growth factor beta Receptor 1 (TGFβRI) protein activity in a cell, including contacting the LOXL2 protein with a LOXL2 generated TGFβRI inhibitor precursor; wherein the LOXL2 generated TGFβRI inhibitor precursor has the formula:

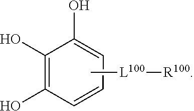

L¹⁰⁰ is a substituted or unsubstituted alkylene, substituted or unsubstituted heteroalkyl ene, substituted or unsubstituted cycloalkylene, substituted or unsubstituted heterocycloalkyl ene, substituted or unsubstituted aryl ene, or substituted or unsubstituted heteroarylene. R¹⁰⁰ is independently halogen, —CX¹⁰⁰₃, —CHX¹⁰⁰₂, —CH₂X¹⁰⁰, —OCH₂X¹⁰⁰, —OCX¹⁰⁰₃, —OCHX¹⁰⁰₂, —N₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol X¹⁰⁰ is —F, —Cl, —Br, or —I In embodiments, the method includes contacting the LOXL2 protein with a LOXL2 generated TGFβRI inhibitor precursor (e.g., LOXL2 generated TGFβRI inhibitor compound precursor), wherein the LOXL2 protein modifies the LOXL2 generated TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound); and wherein the LOXL2 generated TGFβRI inhibitor (e.g., LOXL2 generated TGFβRI inhibitor compound) contacts a TGFβRI protein. In embodiments, the LOXL2 protein is inhibited by the LOXL2 generated TGFβRI inhibitor precursor (e.g., LOXL2 generated TGFβRI inhibitor compound precursor). In embodiments, the LOXL2 protein is inhibited by the LOXL2 reaction converting the LOXL2 generated TGFβRI inhibitor precursor (e.g., LOXL2 generated TGFβRI inhibitor compound precursor) to a TGFβRI inhibitor (e.g., LOXL2 generated TGFβRI inhibitor compound). In embodiments, the inhibition is in a cell expressing LOXL2 and not outside of the cell.

### VI. Methods of Detection

In an aspect is provided a method of detecting inhibition of fibrosis, the method including 1) administering a compound described herein to a subject having fibrosis; 2) measuring the level of pyridinoline (PYD) and/or deoxypyridinoline in a biological sample (e.g., blood or urine of the subject); and detecting the presence of inhibition of fibrosis by detecting a reduction in the level of pyridinoline (PYD) and/or deoxypyridinoline in the biological sample (e.g., blood or urine of the subject).

In an aspect is provided a method of identifying a LOXL2 generated TGFβRI inhibitor precursor (e.g., LOXL2 generated TGFβRI inhibitor compound precursor):

1) capable of contacting LOXL2 protein;
2) capable of being transformed by the LOXL2 protein from a LOXL2 generated TGFβRI inhibitor precursor (e.g., LOXL2 generated TGFβRI inhibitor compound precursor) to a LOXL2 generated TGFβRI inhibitor (e.g., LOXL2 generated TGFβRI inhibitor compound) and wherein the transformation inhibits the LOXL2 protein activity;
3) wherein the LOXL2 generated TGFβRI inhibitor (e.g., LOXL2 generated TGFβRI inhibitor compound) is capable of contacting a TGFβRI protein; and
4) wherein the LOXL2 generated TGFβRI inhibitor (e.g., LOXL2 generated TGFβRI inhibitor compound) inhibits the TGFβRI protein activity.

In embodiments, the LOXL2 protein and TGFβRI protein are in one cell. In embodiments, the LOXL2 generated TGFβRI inhibitor precursor (e.g., LOXL2 generated TGFβRI inhibitor compound precursor) includes a trihydroxyphenol moiety. In embodiments, the LOXL2 generated TGFβRI inhibitor (e.g., LOXL2 generated TGFβRI inhibitor compound) includes a 1-amino-2,3-dihydroxyphenol moiety. In embodiments, the TGFβRI protein activity is kinase activity. In embodiments, the TGFβRI protein activity is cell survival. In embodiments, the TGFβRI protein activity cell proliferation. In embodiments, the TGFβRI protein activity is cell growth. In embodiments, the method includes measuring the level of a detectable agent made by LOXL2 protein activity. In embodiments, the method includes measuring the level of a detectable agent made by TGFβRI protein activity. In embodiments, the method includes measuring the level of kinase activity of TGFβRI protein. In embodiments, the LOXL2 protein and TGFβRI protein are in one vessel. In embodiments, the LOXL2 protein and TGFβRI protein are in one cell. In embodiments, a LOXL2 generated TGFβRI inhibitor precursor (e.g., LOXL2 generated TGFβRI inhibitor compound precursor) is identified from a plurality of test compounds (e.g., a mixture of compounds, one or more of which are LOXL2 generated TGFβRI inhibitor precursors (e.g., LOXL2 generated TGFβRI inhibitor compound precursors) and one or more of which are not LOXL2 generated TGFβRI inhibitor precursors (e.g., LOXL2 generated TGFβRI inhibitor compound precursors) when both LOXL2 protein activity and TGFβRI protein activity are reduced following addition of the LOXL2 generated TGFβRI inhibitor precursor (e.g., LOXL2 generated TGFβRI inhibitor compound precursor) to the vessel or cell.

### EMBODIMENTS

Embodiment P1. A compound having the formula:

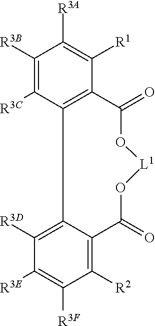

wherein, R¹ is independently hydrogen, halogen, —CX¹₃, —CHX¹₂, —CH₂X¹, —OCX¹₃, —OCH₂X¹, —OCHX¹₂. —CN, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, R²⁰-substituted or unsubstituted C₃-C₄ alkyl, R²⁰-substituted or unsubstituted 2 to 4 membered heteroalkyl; R²⁰ is independently oxo, halogen, —CX²⁰₃, —CHX²⁰₂, —CH₂X²⁰, —OCX²⁰₃, —OCH₂X²⁰, —OCHX²⁰₂, —CN, —SR^{20H}, —ONR^{20E}R^{20F}, —NHC(O)NR^{20E}R^{20F}, —NR^{20E}R^{20F}, —C(O)R^{20G}, —C(O)OR^{20G}, —C(O)NR^{20E}R^{20F}, —OR^{20H}, —NR^{20E}C(O)R^{20G}, —NR^{20E}C(O)OR^{20G}, —NR^{20E}OR^{20G}, unsubstituted C₁-C₆ alkyl, or unsubstituted 2 to 6 membered heteroalkyl; R² is independently hydrogen, halogen, —CX²₃, —CHX²₂, —CH₂X², —OCX²₃, —OCH₂X², —OCHX²₂, —CN, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, R²³-substituted or unsubstituted C₃-C₄ alkyl, or R²³-substituted or unsubstituted 2 to 4 membered heteroalkyl; R²³ is independently oxo, halogen, —CX²³₃, —CHX²³₂, —CH₂X²³, —OCX²³₃, —OCH₂X²³, —OCHX²³₂, —CN, —SR^{23H}, —ONR^{23E}R^{23F}, —NHC(O)NR^{23E}R^{23F}, —NR^{23E}R^{23F}, —C(O)R^{23G}, —C(O)OR^{23G}, —C(O)NR^{23E}R^{23F}, —OR^{23H}, —NR^{23E}C(O)R^{23G}, —NR^{23E}C(O)OR^{23G}, —NR^{23E}OR^{23G}, unsubstituted C₁-C₆ alkyl, or unsubstituted 2 to 6 membered heteroalkyl; R^{3A}, R^{3B}, R^{3C}, R^{3D}, R^{3E}, and R^{3F} are independently hydrogen, —CX³₃, —CN, —OH, —COOH, —CONH₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; L¹ is a R²⁶-substituted C₂-C₉ alkylene, R²⁶-substituted or unsubstituted 2 to 9 membered heteroalkylene, or R²⁶-substituted or unsubstituted C₄-C₆ cycloalkylene; R²⁶ is independently —N₃, —COOR^{26C}, —CONR^{26A}R^{26B}, —NR^{26D}C(O)NR^{26A}R^{26B}, —NR^{26A}C(O)OR^{26C}, or —C(O)R^{26C}; R^{1A}, R^{1B}, R^{1C}, R^{1D}, R^{2A}, R^{2B}, R^{2C}, R^{2D}, R^{20E}, R^{20F}, R^{20G}, R^{20H}, R^{23E}, R^{23F}, R^{23G}, and R^{23H} are independently hydrogen, —CX₃, —CN, —COOH, —CONH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl; R^{1A} and R^{1B} substituents bonded to the same nitrogen atom may optionally be joined to form an unsubstituted 3 to 6 membered heterocycloalkyl or unsubstituted 5 to 6 membered heteroaryl; R^{2A} and R^{2B} substituents bonded to the same nitrogen atom may optionally be joined to form an unsubstituted 3 to 6 membered heterocycloalkyl or unsubstituted 5 to 6 membered heteroaryl; R^{20E} and R^{20F} substituents bonded to the same nitrogen atom may optionally be joined to form an unsubstituted 3 to 6 membered heterocycloalkyl or unsubstituted 5 to 6 membered heteroaryl; R^{23E} and R^{23F} substituents bonded to the same nitrogen atom may optionally be joined to form an unsubstituted 3 to 6 membered heterocycloalkyl or unsubstituted 5 to 6 membered heteroaryl; R^{26A} is independently hydrogen, —CF₃, —CN, —COOH, —CONH₂, R^{27A}-substituted or unsubstituted C₁-C₈ alkyl, R^{27A}-substituted or unsubstituted 2 to 8 membered heteroalkyl, R^{27A}-substituted or unsubstituted C₃-C₈ cycloalkyl, R^{27A}-substituted or unsubstituted 3 to 8 membered heterocycloalkyl, R^{27A}-substituted or unsubstituted C₆-C₁₀ aryl, or R^{27A}-substituted or unsubstituted 5 to 10 membered heteroaryl; R^{26B} is independently hydrogen, —CF₃, —CN, —COOH, —CONH₂, R^{27B}-substituted or unsubstituted C₁-C₈ alkyl, R^{27B}-substituted or unsubstituted 2 to 8 membered heteroalkyl, R^{27B}-substituted or unsubstituted C₃-C₈ cycloalkyl, R^{27B}-substituted or unsubstituted 3 to 8 membered heterocycloalkyl, R^{27B}-substituted or unsubstituted C₆-C₁₀ aryl, or R^{27B}-substituted or unsubstituted 5 to 10 membered heteroaryl; R^{26C} is independently hydrogen, oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, R^{27C}-substituted or unsubstituted C₁-C₈ alkyl, R^{27C}-substituted or unsubstituted 2 to 8 membered heteroalkyl, R^{27C}-substituted or unsubstituted C₃-C₈ cycloalkyl, R^{27C}-substituted or unsubstituted 3 to 8 membered heterocycloalkyl, R^{27C}-substituted or unsubstituted C₆-C₁₀ aryl, or R^{27C}-substituted or unsubstituted 5 to 10 membered heteroaryl; R^{26D} is independently hydrogen, —CF₃, —CN, —COOH, —CONH₂, R^{27D}-substituted or unsubstituted C₁-C₈ alkyl, R^{27D}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27D}-substituted or unsubstituted C₆-C₁₀ aryl, or R^{27D}-substituted or unsubstituted 5 to 10 membered heteroaryl; R^{26A} and R^{26B} substituents bonded to the same nitrogen atom may optionally be joined to form an R^{27A}-substituted or unsubstituted 3 to 8 membered heterocycloalkyl or R^{27A}-substituted or unsubstituted 5 to 10 membered heteroaryl; R^{27A}, R^{27B}, R^{27C}, and R^{27D} are independently oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, —N₃, unsubstituted C₁-C₈ alkyl, unsubstituted 2 to 8 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl; and each X¹, X², X³, X²⁰, and X²³ is independently —F, —Cl, —Br, or —I.

Embodiment P2. The compound of embodiment P1, having the formula:

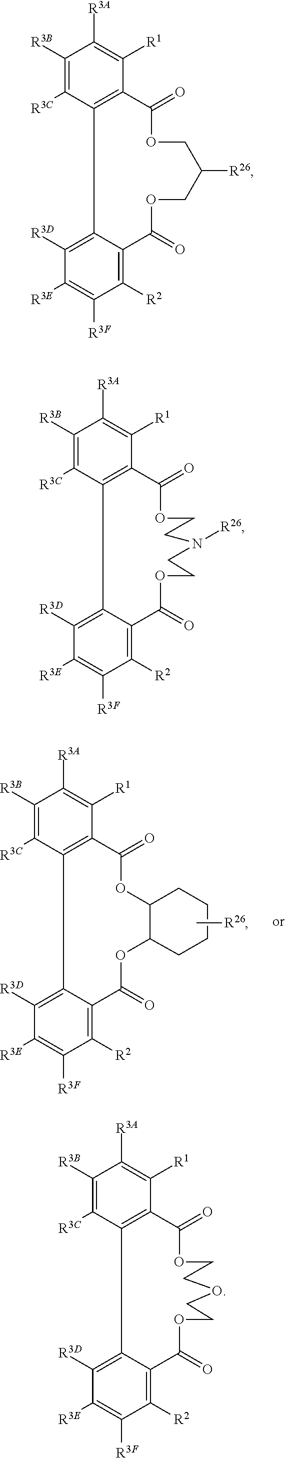

Embodiment P3 The compound of one of embodiments P1 to P2, wherein R^{3A} is —NH₂.

Embodiment P4 The compound of one of embodiments P1 to P2, wherein R^{3A} is —OH.

Embodiment P5. The compound of one of embodiments P1 to P4, wherein R^{3B} is —NH₂.

Embodiment P6. The compound of one of embodiments P1 to P4, wherein R^{3B} is —OH.

Embodiment P7. The compound of one of embodiments P1 to P6, wherein R^{3C} is —NH₂.

Embodiment P8. The compound of one of embodiments P1 to P6, wherein R^{3C} is —OH.

Embodiment P9. The compound of one of embodiments P1 to P8, wherein R^{3D} is —NH₂.

Embodiment P10. The compound of one of embodiments P1 to P8, wherein R^{3D} is —OH.

Embodiment P11. The compound of one of embodiments P1 to P10, wherein R^{3E} is —NH₂.

Embodiment P12. The compound of one of embodiments P1 to P10, wherein R^{3E} is —OH.

Embodiment P13. The compound of one of embodiments P1 to P12, wherein R^{3F} is —NH₂.

Embodiment P14. The compound of one of embodiments P1 to P12, wherein R^{3F} is —OH.

Embodiment P15. The compound of embodiment P1 having the formula:

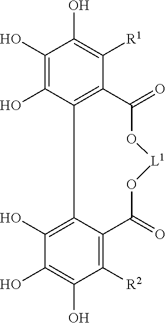

wherein, R¹ is independently hydrogen, halogen, —CX¹₃, —CHX¹₂, —CH₂X¹, —OCX¹₃, —OCH₂X¹, —OCHX¹₂, —CN, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, R²⁰-substituted or unsubstituted C₁-C₄ alkyl, R²⁰-substituted or unsubstituted 2 to 4 membered heteroalkyl; R²⁰ is independently oxo, halogen, —CX²⁰₃, —CHX²⁰₂, —CH₂X²⁰, —OCX²⁰₃, —OCH₂X²⁰, —OCHX²⁰₂, —CN, —SR^{20H}, —ONR^{20E}R^{20F}, —NHC(O)NR^{20E}R^{20F}, —NR^{20E}R^{20F}, —C(O)R^{20G}, —C(O)OR^{20G}, —C(O)NR^{20E}R^{20F}, —OR^{20H}, —NR^{20E}C(O)R^{20G}, —NR^{20E}C(O)OR^{20G}, —NR^{20E}OR^{20G}, unsubstituted C₁-C₆ alkyl, or unsubstituted 2 to 6 membered heteroalkyl; R² is independently hydrogen, halogen, —CX²₃, —CHX²₂, —CH₂X², —OCX²₃, —OCH₂X², —OCHX²₂, —CN, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, R²³-substituted or unsubstituted C₃-C₄ alkyl, or R²³-substituted or unsubstituted 2 to 4 membered heteroalkyl; R²³ is independently oxo, halogen, —CX²³₃, —CHX²³₂, —CH₂X²³, —OCX²³₃, —OCH₂X²³, —OCHX²³₂, —CN, —SR^{23H}, —ONR^{23E}R^{23F}, —NHC(O)NR^{23E}R^{23F}, —NR^{23E}R^{23F}, —C(O)R^{23G}, —C(O)OR^{23G}, —C(O)NR^{23E}R^{23F}, —OR^{23H}, —NR^{23E}C(O)R^{23G}, —NR^{23E}C(O)OR^{23G}, —NR^{23E}OR^{23G}, unsubstituted C₁-C₆ alkyl, or unsubstituted 2 to 6 membered heteroalkyl; L¹ is a R²⁶-substituted C₂-C₉ alkylene, R²⁶-substituted or unsubstituted 2 to 9 membered heteroalkylene, or R²⁶-substituted or unsubstituted C₄-C₆ cycloalkylene; R²⁶ is independently —N₃, —COOR^{26C}, —CONR^{26A}R^{26B}, —NR^{26D}C(O)NR^{26A}R^{26B}, —NR^{26A}C(O)OR^{26C}, or —C(O)R^{26C}; R^{1A}, R^{1B}, R^{1C}, R^{1D}, R^{2A}, R^{2B}, R^{2C}, R^{2D}, R^{20E}, R^{20F}, R^{20G}, R^{20H}, R^{23E}, R^{23F}, R^{23G}, and R^{23H} are independently hydrogen, —CX₃, —CN, —COOH, —CONH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl; R^{1A} and R^{1B} substituents bonded to the same nitrogen atom may optionally be joined to form an unsubstituted 3 to 6 membered heterocycloalkyl or unsubstituted 5 to 6 membered heteroaryl; R^{2A} and R^{2B} substituents bonded to the same nitrogen atom may optionally be joined to form an unsubstituted 3 to 6 membered heterocycloalkyl or unsubstituted 5 to 6 membered heteroaryl; R^{20E} and R^{20F} substituents bonded to the same nitrogen atom may optionally be joined to form an unsubstituted 3 to 6 membered heterocycloalkyl or unsubstituted 5 to 6 membered heteroaryl; R^{23E} and R^{23F} substituents bonded to the same nitrogen atom may optionally be joined to form an unsubstituted 3 to 6 membered heterocycloalkyl or unsubstituted 5 to 6 membered heteroaryl; R^{26A} is independently hydrogen, —CF₃, —CN, —COOH, —CONH₂, R^{27A}-substituted or unsubstituted C₁-C₈ alkyl, R^{27A}-substituted or unsubstituted 2 to 8 membered heteroalkyl, R^{27A}-substituted or unsubstituted C₃-C₈ cycloalkyl, R^{27A}-substituted or unsubstituted 3 to 8 membered heterocycloalkyl, R^{27A}-substituted or unsubstituted C₆-C₁₀ aryl, or R^{27A}-substituted or unsubstituted 5 to 10 membered heteroaryl; R^{26B} is independently hydrogen, —CF₃, —CN, —COOH, —CONH₂, R^{27B}-substituted or unsubstituted C₁-C₈ alkyl, R^{27B}-substituted or unsubstituted 2 to 8 membered heteroalkyl, R^{27B}-substituted or unsubstituted C₃-C₈ cycloalkyl, R^{27B}-substituted or unsubstituted 3 to 8 membered heterocycloalkyl, R^{27B}-substituted or unsubstituted C₆-C₁₀ aryl, or R^{27B}-substituted or unsubstituted 5 to 10 membered heteroaryl; R^{26C} is independently hydrogen, oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, R^{27C}-substituted or unsubstituted C₁-C₈ alkyl, R^{27C}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27C}-substituted or unsubstituted C₆-C₁₀ aryl, or R^{27C}-substituted or unsubstituted 5 to 10 membered heteroaryl; R^{26D} is independently hydrogen, —CF₃, —CN, —COOH, —CONH₂, R^{27D}-substituted or unsubstituted C₁-C₈ alkyl, R^{27D}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27D}-substituted or unsubstituted C₆-C₁₀ aryl, or R^{27D}-substituted or unsubstituted 5 to 10 membered heteroaryl; R^{26A} and R^{26B} substituents bonded to the same nitrogen atom may optionally be joined to form an R^{27A}-substituted or unsubstituted 3 to 8 membered heterocycloalkyl or R^{27A}-substituted or unsubstituted 5 to 10 membered heteroaryl; R^{27A}, R^{27B}, R^{27C}, and R^{27D} are independently oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, —N₃, unsubstituted C₁-C₈ alkyl, unsubstituted 2 to 8 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl; and each X¹, X², X²⁰, and X²³ is independently-F, —Cl, —Br, or —I.

Embodiment P16. The compound of embodiment P15 having the formula:

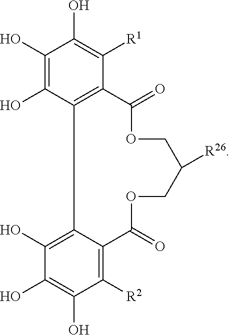

Embodiment P17. The compound of embodiment P15 having the formula:

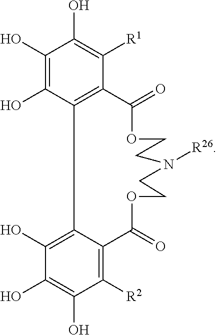

Embodiment P18. The compound of embodiment P15 having the formula:

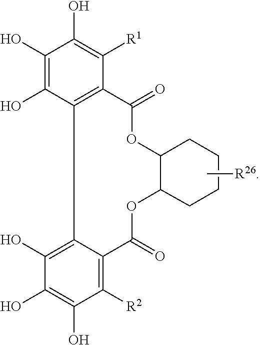

Embodiment P19. The compound of embodiment P15 having the formula:

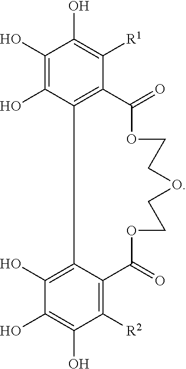

Embodiment P20. The compound of one of embodiments P1 to P18, wherein R²⁶ is independently —COOR^{26C}, —NR^{26A}C(O)OR^{26C}, or —C(O)R^{26C}.

Embodiment P21. The compound of one of embodiments P1 to P18, wherein R²⁶ is independently —COOR^{26C}.

Embodiment P22. The compound of one of embodiments P1 to P18, wherein R²⁶ is independently —NR^{26A}C(O)OR^{26C}.

Embodiment P23. The compound of one of embodiments P1 to P18, wherein R²⁶ is independently —C(O)R^{26C}.

Embodiment P24. The compound of one of embodiments P1 to P23, wherein R^{26A} is independently unsubstituted C₁-C₄ alkyl or R^{27A}-substituted or unsubstituted phenyl; R^{26B} is independently unsubstituted C₁-C₄ alkyl or R^{27B}-substituted or unsubstituted phenyl; R^{26C} is independently unsubstituted C₁-C₄ alkyl or R^{27C}-substituted or unsubstituted phenyl; and R^{26D} is independently unsubstituted C₁-C₄ alkyl or R^{27D}-substituted or unsubstituted phenyl.

Embodiment P25. The compound of one of embodiments P1 to P23, wherein R^{26A}, R^{26B}, R^{26C}, and R^{26D} are independently unsubstituted C₁-C₄ alkyl.

Embodiment P26. The compound of one of embodiments P1 to P23, wherein
R^{26A} is independently R^{27A}-substituted or unsubstituted phenyl;
R^{26B} is independently R^{27B}-substituted or unsubstituted phenyl;
R^{26C} is independently R^{27C}-substituted or unsubstituted phenyl; and
R^{26D} is independently R^{27D}-substituted or unsubstituted phenyl.

Embodiment P27. The compound of one of embodiments P1 to P23, wherein R^{26A}, R^{26B}, R^{26C}, and R^{26D} are independently hydroxyl-substituted phenyl.

Embodiment P28. The compound of one of embodiments P1 to P23, wherein R^{26A}, R^{26B}, R^{26C}, and R^{26D} are independently unsubstituted phenyl.

Embodiment P29. The compound of one of embodiments P1 to P28, wherein R¹ is independently hydrogen.

Embodiment P30. The compound of one of embodiments P1 to P28, wherein R² is independently hydrogen.

Embodiment P31. A pharmaceutical composition comprising the compound of any one of embodiments P1 to P30 and a pharmaceutically acceptable excipient.

Embodiment P32. A method of reducing the level of activity of zinc finger protein Snail1 in a subject, said method comprising administering an effective amount of a compound of one of embodiments P1 to P30 to the subject.

Embodiment P33. A method of reducing the level of activity of Lysyl oxidase homolog 2 in a subject, said method comprising administering an effective amount of a compound of one of embodiments P1 to P30 to the subject.

Embodiment P34. A method of inhibiting collagen cross-linking in a subject, said method comprising administering an effective amount of a compound of one of embodiments P1 to P30 to the subject.

Embodiment P35. A method of treating fibrosis, said method comprising administering to a subject in need thereof an effective amount of a compound of one of embodiments P15 to P30.

Embodiment P36. A method of treating pulmonary fibrosis, said method comprising administering to a subject in need thereof an effective amount of a compound of one of embodiments P1 to P30.

Embodiment P37. A method of treating idiopathic pulmonary fibrosis, said method comprising administering to a subject in need thereof an effective amount of a compound of one of embodiments P1 to P30.

Embodiment P38. A method of treating cancer, said method comprising administering to a subject in need thereof an effective amount of a compound of one of embodiments P1 to P30.

Embodiment P39. A method of treating cancer metastasis, said method comprising administering to a subject in need thereof an effective amount of a compound of one of embodiments P1 to P30.

### Additional Embodiments

Embodiment 1. A compound having the formula:

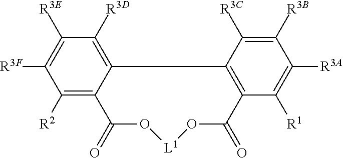

wherein,

R¹ is independently hydrogen, halogen, —CX¹₃, —CHX¹₂, —CH₂X¹, —OCX¹₃, —OCH₂X¹, —OCHX¹₂, —CN, —N₃, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, R²⁰-substituted or unsubstituted C₁-C₄ alkyl, R²⁰-substituted or unsubstituted 2 to 4 membered heteroalkyl; R²⁰ is independently oxo, halogen, —CX²⁰₃, —CHX²⁰₂, —CH₂X²⁰, —OCX²⁰₃, —OCH₂X²⁰, —OCHX²⁰₂, —CN, —N₃, —SR^{20H}, —ONR^{20E}R^{20F}, —NHC(O)NR^{20E}R^{20F}, —NR^{20E}R^{20F}, —C(O)R^{20G}, —C(O)OR^{20G}, —C(O)NR^{20E}R^{20F}, —OR^{20H}, —NR^{20E}C(O)R^{20G}, —NR^{20E}C(O)OR^{20G}, —NR^{20E}OR^{20G}, unsubstituted C₁-C₆ alkyl, or unsubstituted 2 to 6 membered heteroalkyl; R² is independently hydrogen, halogen, —CX²₃, —CHX²₂, —CH₂X², —OCX²₃, —OCH₂X², —OCHX²₂, —CN, —N₃, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, R²³-substituted or unsubstituted C₃-C₄ alkyl, or R²³-substituted or unsubstituted 2 to 4 membered heteroalkyl; R²³ is independently oxo, halogen, —CX²³₃, —CHX²³₂, —CH₂X²³, —OCX²³₃, —OCH₂X²³, —OCHX²³₂, —CN, —N₃, —SR^{23H}, —ONR^{23B}R^{23F}, —NHC(O)NR^{23E}R^{23F}, —NR^{23E}R^{23F}, —C(O)R^{23G}, —C(O)OR^{23G}, —C(O)NR^{23E}R^{23F}, —OR^{23H}, —NR^{23E}C(O)R^{23G}, —NR^{23E}C(O)OR^{23G}, —NR^{23E}OR^{23G}, unsubstituted C₁-C₆ alkyl, or unsubstituted 2 to 6 membered heteroalkyl; R^{3A}, R^{3B}, R^{3C}, R^{3D}, R^{3E}, and R^{3F} are independently —OH, —NH₂, hydrogen, —CX³₃, —CN, —N₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; L¹ is a R²⁶-substituted C₂-C₁₀ alkylene, R²⁶-substituted or unsubstituted 2 to 9 membered heteroalkylene, or R²⁶-substituted or unsubstituted C₄-C₆ cycloalkylene; R²⁶ is independently —N₃, —COOR^{26C}, —CONR^{26A}R^{26B}, —NR^{26D}C(O)NR^{26A}R^{26B}, —NR^{26A}C(O)OR^{26C}, or —C(O)R^{26C}; R^{1A}, R^{1B}, R^{1C}, R^{1D}, R^{2A}, R^{2B}, R^{2C}, R^{2D}, R^{20E}, R^{20F}, R^{20G}, R^{20H}, R^{23E}, R^{23F}, R^{23G} and R^{23H} are independently hydrogen, —CX₃, —CN, —COOH, —CONH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl; R^{1A} and R^{1B} substituents bonded to the same nitrogen atom may optionally be joined to form an unsubstituted 3 to 6 membered heterocycloalkyl or unsubstituted 5 to 6 membered heteroaryl; R^{2A} and R^{2B} substituents bonded to the same nitrogen atom may optionally be joined to form an unsubstituted 3 to 6 membered heterocycloalkyl or unsubstituted 5 to 6 membered heteroaryl; R^{20E} and R^{20F} substituents bonded to the same nitrogen atom may optionally be joined to form an unsubstituted 3 to 6 membered heterocycloalkyl or unsubstituted 5 to 6 membered heteroaryl; R^{23E} and R^{23F} substituents bonded to the same nitrogen atom may optionally be joined to form an unsubstituted 3 to 6 membered heterocycloalkyl or unsubstituted 5 to 6 membered heteroaryl; R^{26A} is independently hydrogen, —CF₃, —CN, —COOH, —CONH₂, R^{27A}-substituted or unsubstituted C₁-C₈ alkyl, R^{27A}-substituted or unsubstituted 2 to 8 membered heteroalkyl, R^{27A}-substituted or unsubstituted C₃-C₈ cycloalkyl, R^{27A}-substituted or unsubstituted 3 to 8 membered heterocycloalkyl, R^{27A}-substituted or unsubstituted C₆-C₁₀ aryl, or R^{27A}-substituted or unsubstituted 5 to 10 membered heteroaryl; R^{26B} is independently hydrogen, —CF₃, —CN, —COOH, —CONH₂, R^{27B}-substituted or unsubstituted C₁-C₈ alkyl, R^{27B}-substituted or unsubstituted 2 to 8 membered heteroalkyl, R^{27B}-substituted or unsubstituted C₃-C₈ cycloalkyl, R^{27B}-substituted or unsubstituted 3 to 8 membered heterocycloalkyl, R^{27B}-substituted or unsubstituted C₆-C₁₀ aryl, or R^{27B}-substituted or unsubstituted 5 to 10 membered heteroaryl; R^{26C} is independently hydrogen, oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, R^{27C}-substituted or unsubstituted C₁-C₈ alkyl, R^{27C}-substituted or unsubstituted 2 to 8 membered heteroalkyl, R^{27C}-substituted or unsubstituted C₃-C₈ cycloalkyl, R^{27C}-substituted or unsubstituted 3 to 8 membered heterocycloalkyl, R^{27C}-substituted or unsubstituted C₆-C₁₀ aryl, or R^{27C}-substituted or unsubstituted 5 to 10 membered heteroaryl; R^{26D} is independently hydrogen, —CF₃, —CN, —COOH, —CONH₂, R^{27D}-substituted or unsubstituted C₃-C₈ alkyl, R^{27D}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27D}-substituted or unsubstituted C₆-C₁₀ aryl, or R^{27D}-substituted or unsubstituted 5 to 10 membered heteroaryl; R^{26A} and R^{26B} substituents bonded to the same nitrogen atom may optionally be joined to form an R^{27A}-substituted or unsubstituted 3 to 8 membered heterocycloalkyl or R^{27A}-substituted or unsubstituted 5 to 10 membered heteroaryl; R^{27A}, R^{27B}, R^{27C}, and R^{27D} are independently oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, —N₃, unsubstituted C₁-C₈ alkyl, unsubstituted 2 to 8 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl; wherein at least one of the R^{3A}, R^{3B}, R^{3C}, R^{3D}, R^{3E}, or R^{3F} substituents are independently —OH; and each X¹, X², X³, X²⁰, and X²³ is independently —F, —Cl, —Br, or —I.

Embodiment 2. The compound of embodiment 1, having the formula:

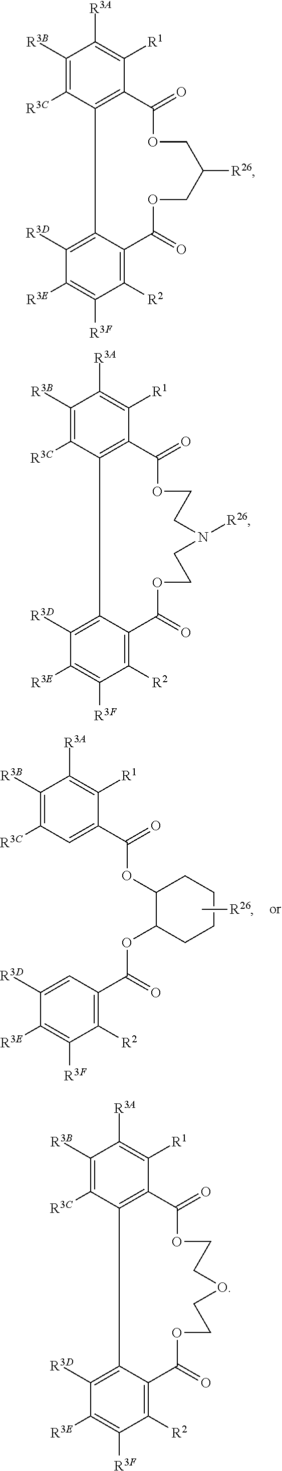

Embodiment 3. The compound of one of embodiments 1 to 2, wherein R^{3A} is —NH₂.

Embodiment 4. The compound of one of embodiments 1 to 2, wherein R^{3A} is —OH.

Embodiment 5. The compound of one of embodiments 1 to 4, wherein R^{3B} is —NH₂.

Embodiment 6. The compound of one of embodiments 1 to 4, wherein R^{3B} is —OH.

Embodiment 7. The compound of one of embodiments 1 to 6, wherein R^{3C} is —NH₂.

Embodiment 8. The compound of one of embodiments 1 to 6, wherein R^{3C} is —OH.

Embodiment 9. The compound of one of embodiments 1 to 8, wherein R^{3D} is —NH₂.

Embodiment 10. The compound of one of embodiments 1 to 8, wherein R^{3D} is —OH.

Embodiment 11. The compound of one of embodiments 1 to 10, wherein R^{3E} is —NH₂.

Embodiment 12. The compound of one of embodiments 1 to 10, wherein R^{3E} is —OH.

Embodiment 13. The compound of one of embodiments 1 to 12, wherein R^{3F} is —NH₂.

Embodiment 14. The compound of one of embodiments 1 to 12, wherein R^{3F} is —OH.

Embodiment 15. The compound of embodiment 1 having the formula:

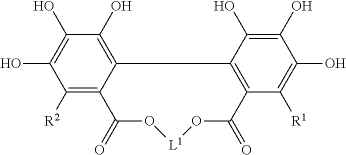

wherein, R¹ is independently hydrogen, halogen, —CX¹₃, —CHX¹₂, —CH₂X¹, —OCX¹₃, —OCH₂X¹, —OCHX¹₂, —CN, —N₃, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, R²⁰-substituted or unsubstituted C₃-C₄ alkyl, R²⁰-substituted or unsubstituted 2 to 4 membered heteroalkyl; R²⁰ is independently oxo, halogen, —CX²⁰₃, —CHX²⁰₂, —CH₂X²⁰, —OCX²⁰₃, —OCH₂X²⁰, —OCHX²⁰₂, —CN, —N₃, —SR^{20H}, —ONR^{20E}R^{20F}, —NHC(O)NR^{20E}R^{20F}, —NR^{20E}R^{20F}, —C(O)R^{20G}, —C(O)OR^{20G}, —C(O)NR^{20E}R^{20F}, —OR^{20H}, —NR^{20E}C(O)R^{20G}, —NR^{20E}C(O)OR^{20G}, —NR^{20E}OR^{20G}, unsubstituted C₁-C₆ alkyl, or unsubstituted 2 to 6 membered heteroalkyl; R² is independently hydrogen, halogen, —CX²₃, —CHX²₂, —CH₂X², —OCX²₃, —OCH₂X², —OCHX²₂, —CN, —N₃, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, R²³-substituted or unsubstituted C₃-C₄ alkyl, or R²³-substituted or unsubstituted 2 to 4 membered heteroalkyl; R²³ is independently oxo, halogen, —CX²³₃, —CHX²³₂, —CH₂X²³, —OCX²³₃, —OCH₂X²³, —OCHX²³₂, —CN, —N₃, —SR^{23H}, —ONR^{23B}R^{23F}, —NHC(O)NR^{23E}R^{23F}, —NR^{23E}R^{23F}, —C(O)R^{23G}, —C(O)OR^{23G}, —C(O)NR^{23E}R^{23F}, —OR^{23H}, —NR^{23E}C(O)R^{23G}, —NR^{23E}C(O)OR^{23G}, —NR^{23E}OR^{23G}, unsubstituted C₁-C₆ alkyl, or unsubstituted 2 to 6 membered heteroalkyl; Lisa R²⁶-substituted C₂-C₉ alkylene, R²⁶-substituted or unsubstituted 2 to 9 membered heteroalkylene, or R²⁶-substituted or unsubstituted C₄-C₆ cycloalkylene; R²⁶ is independently —N₃, —COOR^{26C}, —CONR^{26A}R^{26B}, —NR^{26D}C(O)NR^{26A}R^{26B}, —NR^{26A}C(O)OR^{26C}, or —C(O)R^{26C}; R^{1A}, R^{1B}, R^{1C}, R^{1D}, R^{2A}, R^{2B}, R^{2C}, R^{2D}, R^{20E}, R^{20F}, R^{20G}, R^{20H}, R^{23E}, R^{23F}, R^{23G}, and R^{23H} are independently hydrogen, —CX₃, —CN, —COOH, —CONH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl; R^{1A} and R^{1B} substituents bonded to the same nitrogen atom may optionally be joined to form an unsubstituted 3 to 6 membered heterocycloalkyl or unsubstituted 5 to 6 membered heteroaryl; R^{2A} and R^{2B} substituents bonded to the same nitrogen atom may optionally be joined to form an unsubstituted 3 to 6 membered heterocycloalkyl or unsubstituted 5 to 6 membered heteroaryl; R^{20E} and R^{20F} substituents bonded to the same nitrogen atom may optionally be joined to form an unsubstituted 3 to 6 membered heterocycloalkyl or unsubstituted 5 to 6 membered heteroaryl; R^{23E} and R^{23F} substituents bonded to the same nitrogen atom may optionally be joined to form an unsubstituted 3 to 6 membered heterocycloalkyl or unsubstituted 5 to 6 membered heteroaryl; R^{26A} is independently hydrogen, —CF₃, —CN, —COOH, —CONH₂, R^{27A}-substituted or unsubstituted C₁-C₈ alkyl, R^{27A}-substituted or unsubstituted 2 to 8 membered heteroalkyl, R^{27A}-substituted or unsubstituted C₃-C₈ cycloalkyl, R^{27A}-substituted or unsubstituted 3 to 8 membered heterocycloalkyl, R^{27A}-substituted or unsubstituted C₆-C₁₀ aryl, or R^{27A}-substituted or unsubstituted 5 to 10 membered heteroaryl; R^{26B} is independently hydrogen, —CF₃, —CN, —COOH, —CONH₂, R^{27B}-substituted or unsubstituted C₁-C₈ alkyl, R^{27B}-substituted or unsubstituted 2 to 8 membered heteroalkyl, R^{27B}-substituted or unsubstituted C₃-C₈ cycloalkyl, R^{27B}-substituted or unsubstituted 3 to 8 membered heterocycloalkyl, R^{27B}-substituted or unsubstituted C₅-C₁₀ aryl, or R^{27B}-substituted or unsubstituted 5 to 10 membered heteroaryl; R^{26C} is independently hydrogen, oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, R^{27C}-substituted or unsubstituted C₁-C₈ alkyl, R^{27C}-substituted or unsubstituted 2 to 8 membered heteroalkyl, R^{27C}-substituted or unsubstituted C₃-C₈ cycloalkyl, R^{27C}-substituted or unsubstituted 3 to 8 membered heterocycloalkyl, R^{27C}-substituted or unsubstituted C₆-C₁₀ aryl, or R^{27C}-substituted or unsubstituted 5 to 10 membered heteroaryl; R^{26D} is independently hydrogen, —CF₃, —CN, —COOH, —CONH₂, R^{27D}-substituted or unsubstituted C₁-C₈ alkyl, R^{27D}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27D}-substituted or unsubstituted C₆-C₁₀ aryl, or R^{27D}-substituted or unsubstituted 5 to 10 membered heteroaryl; R^{26A} and R^{26B} substituents bonded to the same nitrogen atom may optionally be joined to form an R^{27A}-substituted or unsubstituted 3 to 8 membered heterocycloalkyl or R^{27A}-substituted or unsubstituted 5 to 10 membered heteroaryl; R^{27A}, R^{27B}, R^{27C}, and R^{27D} are independently oxo, halogen, —CF₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, —N₃, unsubstituted C₁-C₈ alkyl, unsubstituted 2 to 8 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl; and each X¹, X², X²⁰, and X²³ is independently —F, —Cl, —Br, or —I.

Embodiment 16. The compound of embodiment 15 having the formula:

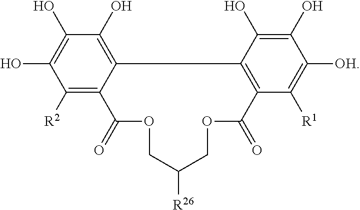

Embodiment 17. The compound of embodiment 15 having the formula:

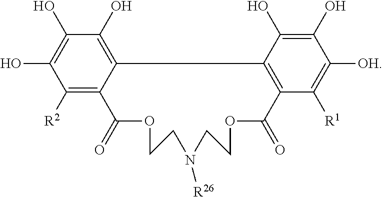

Embodiment 18. The compound of embodiment 15 having the formula:

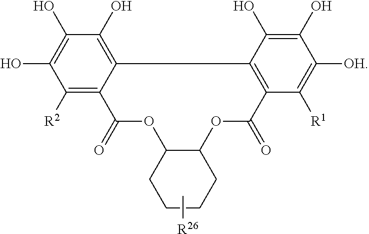

Embodiment 19. The compound of embodiment 15 having the formula:

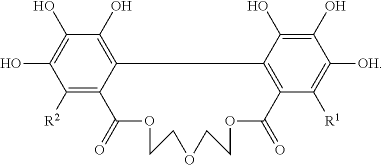

Embodiment 20. The compound of one of embodiments 1 to 18, wherein R²⁶ is independently —COOR^{26C}, —NR^{26A}C(O)OR^{26C}, or —C(O)R^{26C}.

Embodiment 21. The compound of one of embodiments 1 to 18, wherein R²⁶ is independently —COOR^{26C}.

Embodiment 22. The compound of one of embodiments 1 to 18, wherein R²⁶ is independently —NR^{26A}C(O)OR^{26C}.

Embodiment 23. The compound of one of embodiments 1 to 18, wherein R²⁶ is independently —C(O)R^{26C}.

Embodiment 24. The compound of one of embodiments 1 to 23, wherein R^{26A} is independently unsubstituted C₁-C₄ alkyl or R^{27A}-substituted or unsubstituted phenyl; R^{26B} is independently unsubstituted C₁-C₄ alkyl or R^{27B}-substituted or unsubstituted phenyl; R^{26B} is independently unsubstituted C₁-C₄ alkyl or R^{27C}-substituted or unsubstituted phenyl; and R^{26D} is independently unsubstituted C₁-C₄ alkyl or R^{27D}-substituted or unsubstituted phenyl.

Embodiment 25. The compound of one of embodiments 1 to 23, wherein R^{26A}, R^{26B}, R^{26C}, and R^{26D} are independently unsubstituted C₁-C₄ alkyl.

Embodiment 26. The compound of one of embodiments 1 to 23, wherein R^{26A} is independently R^{27A}-substituted or unsubstituted phenyl; R^{26B} is independently R^{27B}-substituted or unsubstituted phenyl; R^{26C} is independently R^{27C}-substituted or unsubstituted phenyl; and R^{26D} is independently R^{27D}-substituted or unsubstituted phenyl.

Embodiment 27. The compound of one of embodiments 1 to 23, wherein R^{26A}, R^{26B}, R^{26C}, and R^{26D} are independently hydroxyl-substituted phenyl.

Embodiment 28. The compound of one of embodiments 1 to 23, wherein R^{26A}, R^{26B}, R^{26C}, and R^{26D} are independently unsubstituted phenyl.

Embodiment 29. The compound of one of embodiments 1 to 28, wherein R¹ is independently hydrogen.

Embodiment 30. The compound of one of embodiments 1 to 28, wherein R² is independently hydrogen.

Embodiment 31. A pharmaceutical composition comprising the compound of any one of embodiments 1 to 30 and a pharmaceutically acceptable excipient.

Embodiment 32. A method of reducing the level of activity of zinc finger protein Snail1 in a subject, said method comprising administering an effective amount of a compound of one of embodiments 1 to 30 to the subject.

Embodiment 33. A method of reducing the level of activity of Lysyl oxidase homolog 2 (LOXL2) in a subject, said method comprising administering an effective amount of a compound of one of embodiments 1 to 30 to the subject.

Embodiment 34. A method of inhibiting collagen cross-linking in a subject, said method comprising administering an effective amount of a compound of one of embodiments 1 to 30 to the subject.

Embodiment 35. A method of treating fibrosis, said method comprising administering to a subject in need thereof an effective amount of a compound of one of embodiments 15 to 30.

Embodiment 36. A method of treating pulmonary fibrosis, said method comprising administering to a subject in need thereof an effective amount of a compound of one of embodiments 1 to 30.

Embodiment 37. A method of treating idiopathic pulmonary fibrosis, said method comprising administering to a subject in need thereof an effective amount of a compound of one of embodiments 1 to 30.

Embodiment 38. A method of treating cancer, said method comprising administering to a subject in need thereof an effective amount of a compound of one of embodiments 1 to 30.

Embodiment 39. A method of treating cancer metastasis, said method comprising administering to a subject in need thereof an effective amount of a compound of one of embodiments 1 to 30.

Embodiment 40. A method of inhibiting Lysyl oxidase homolog 2 (LOXL2) protein activity and Transforming growth factor beta Receptor 1 (TGFβRI) protein activity in a cell, comprising contacting the LOXL2 protein with a LOXL2 generated TGFβRI inhibitor precursor; wherein the LOXL2 generated TGFβRI inhibitor precursor has the formula:

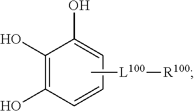

wherein L¹⁰⁰ is a substituted or unsubstituted alkylene, substituted or unsubstituted heteroalkylene, substituted or unsubstituted cycloalkylene, substituted or unsubstituted heterocycloalkyl ene, substituted or unsubstituted aryl ene, or substituted or unsubstituted heteroarylene; R¹⁰⁰ is independently halogen, —CX¹⁰⁰₃, —CHX¹⁰⁰₂, —CH₂X¹⁰⁰, —OCH₂X¹⁰⁰, —OCX¹⁰⁰₃, —OCHX¹⁰⁰₂, —N₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and X¹⁰⁰ is —F, —Cl, —Br, or —I.

### VII. Examples

TGFβ 1 signaling is a key driver of collagen accumulation and fibrotic disease but also an important regulator of inflammation and epithelial proliferation^{1,2}. Trihydroxyphenolic motifs, found in ellagitannins such as corilagin, are have been identified as potent (IC50˜50 nM) blockers of TGFβ1 signaling, Snail1 expression, and collagen accumulation. Compounds effective in vivo in models of pulmonary fibrosis and collagen-dependent lung cancer metastasis have been identified. Remarkably, the functional effects of trihydroxyphenolic compounds is related to the presence of active lysyl oxidase-like 2 (LOXL2). Effects of such compounds are most evident in fibroblasts or transformed cells, the major LOXL2 producers^{3,4}. Oxidation of trihydroxyphenolic motifs by LOXL2 to a 3-aminobenzene-1,2-diol (3Abd)-like metabolite by the LOXL2/3 specific lysine 731 as the N donor and resulting in an inactivating internal allysine was observed. 3Abd was found to directly inhibit TGFβR1 kinase activity. Combined inhibition of LOXL2 and TGFβR1 kinase resulted in potent in vivo anti-fibrotic activity that could be tracked by urinary levels of collagen crosslinks. We have thus identified a new anti-fibrotic approach to inhibit TGFβ1 signaling restricted to fibroblast-like cells. Moreover, the findings uncover a biological pathway for disease prevention by polyphenols such as epigallocatechin-3-gallate, the major polyphenol in green tea, that operates at plasma levels achievable by ingestion⁵. This pathway may bridge the longstanding discrepancy between disease-attenuating activity of polyphenols implied by population studies and the levels of polyphenols previously required for in vitro bioactivity largely unachievable in humans ingesting these agents^{6,7}. The biological pathway identified here employs trihydroxyphenolic polyphenols at concentrations achievable by dietary ingestion. This contrasts with the large body of prior studies that have employed μM levels of polyphenols to achieve in vitro “anti-oxidant” or signaling inhibition in multiple cell systems²⁶ even though blood levels above ˜150 nM have not been documented for dietary polyphenols consumed by humans^{5,7,27-29}. The pathway we describe may be the first example of a relevant protective pathway activated by nM levels of the trihydroxyphenol subclass, possibly contributing to the observed beneficial effects of green tea and other trihydroxyphenolic-rich diets in numerous population studies^{6,30,31}.

### Example 1. Small Molecule LOXL2 Inhibitors to Block Pulmonary Fibrosis and Cancer Progression

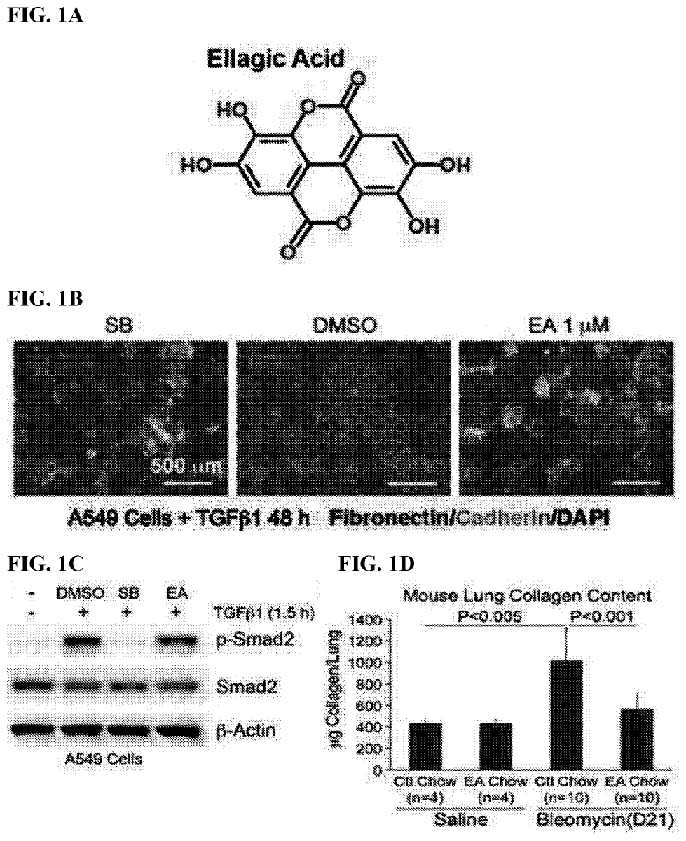

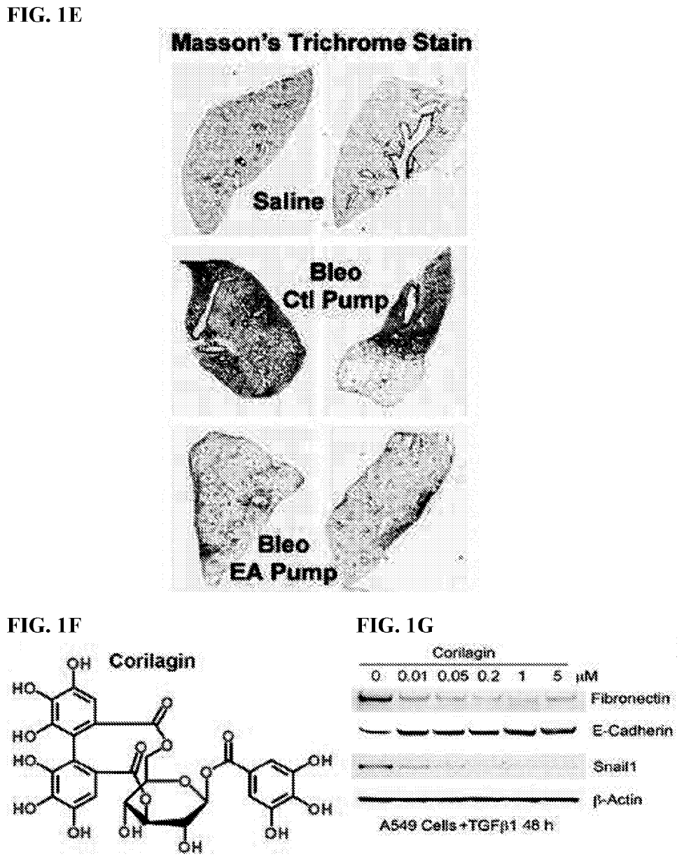

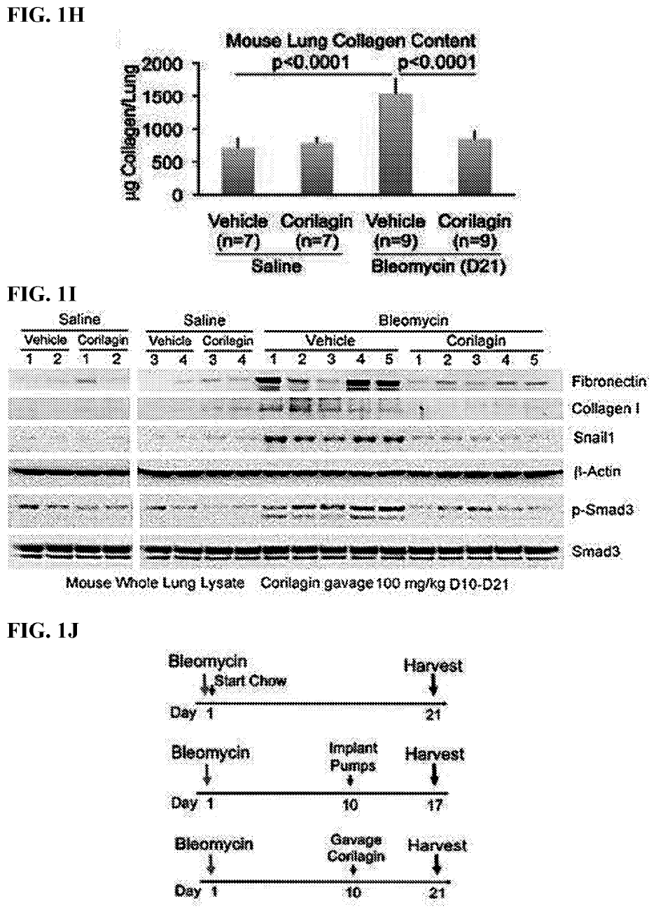

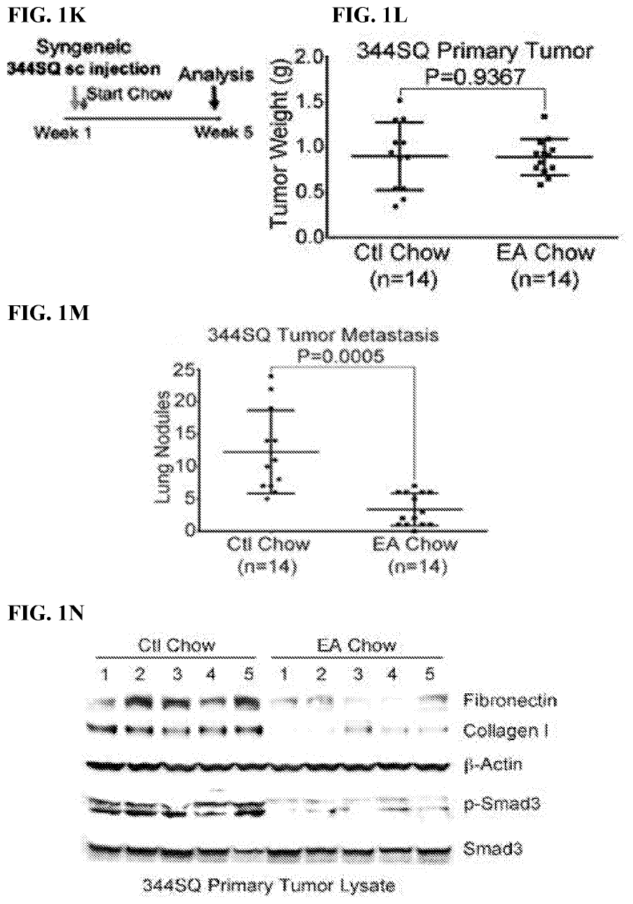

IPF is driven by TGFβ1-driven disposition of extracellular matrix proteins from myofibroblasts. The investigators performed a high-throughput screen and identified compounds as potent inhibitors of Snail1 expression, a marker of TGBβ1 signaling. These compounds inhibit TGFβ signaling in lung fibroblasts and lung cancer cells but not in lung epithelial cells, indicating promise for less toxicity as compared to other available TGFβ inhibitors. Compounds as described herein thus have potential to be oral anti-fibrotic drugs, for example the compounds depicted in FIGS. 1A-1I.

Subsequent mechanistic studies have elucidated the mechanism by which induced TGFβ1 inhibition reduces fibrosis: in the presence of tested compounds, TGFβ1 no longer stimulates lysyl oxidase-like 2 (LOXL2). LOXL2 stabilizes intracellular collagen transcription and extracellular collagen cross-linking. Inhibiting collagen through both intracellular and extracellular mechanisms may by superior to other LOXL2 inhibitors. Compounds described herein are being tested for TGFβ1 inhibition and reduction of fibrosis.

Compounds as described herein are developed to block collagen accumulation in IPF and other fibrotic disorders (e.g. cirrhosis) as well as in tumors characterized by excessive stromal accumulation.

### Example 2. Identification of Compounds Capable of Reducing Collagen Accumulation

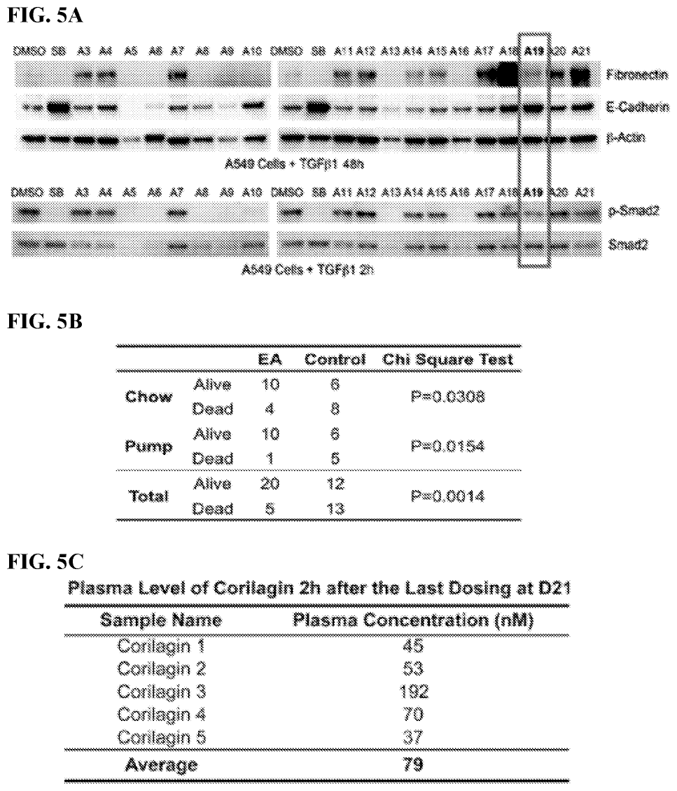

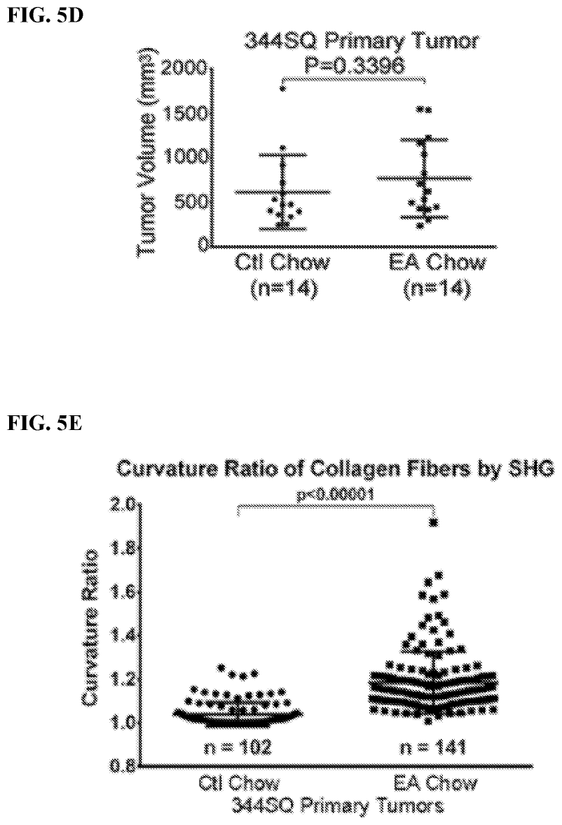

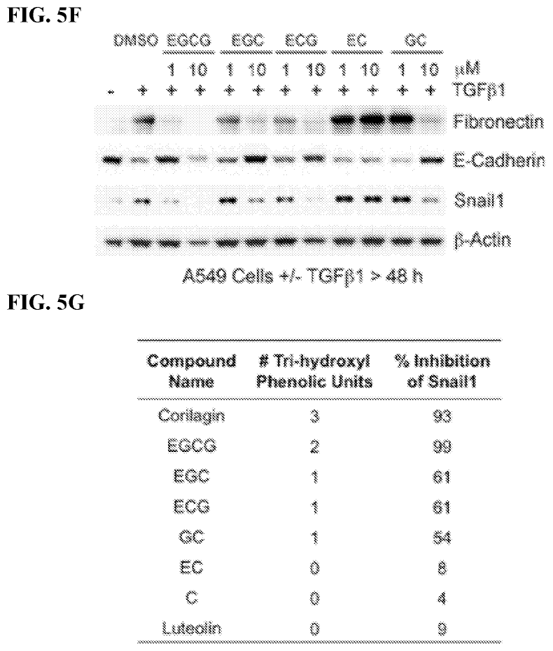

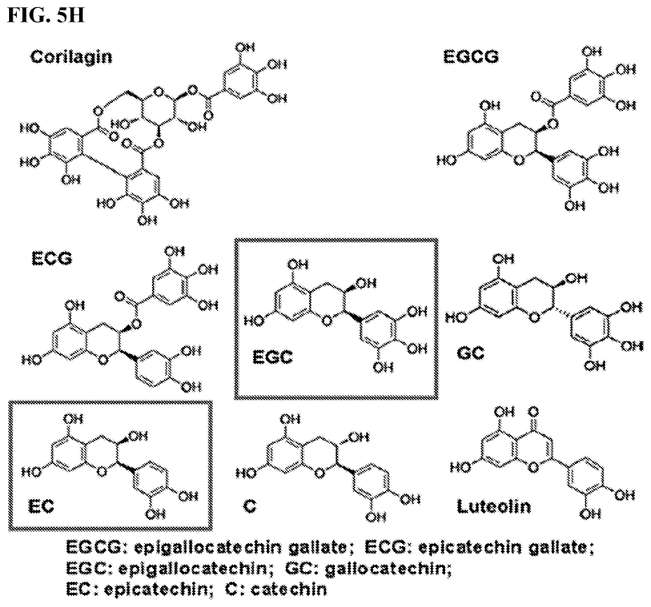

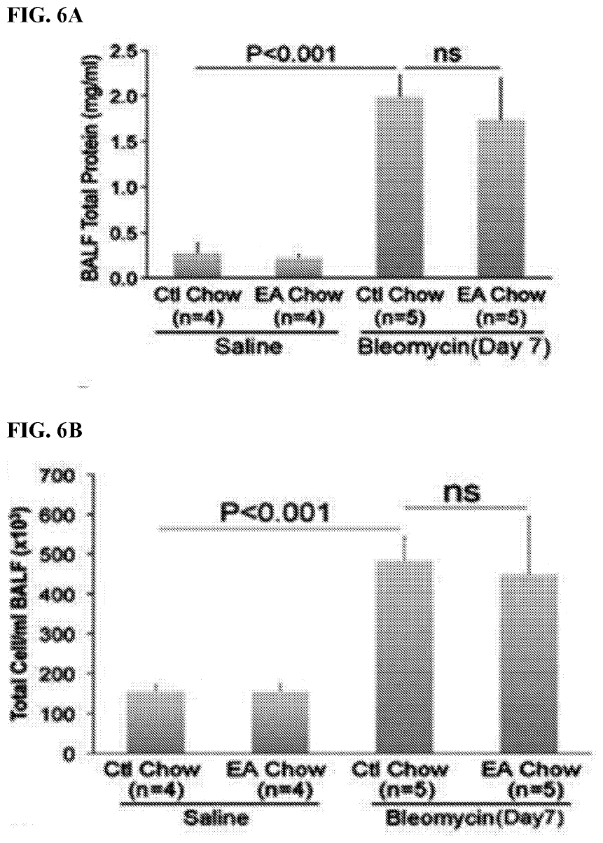

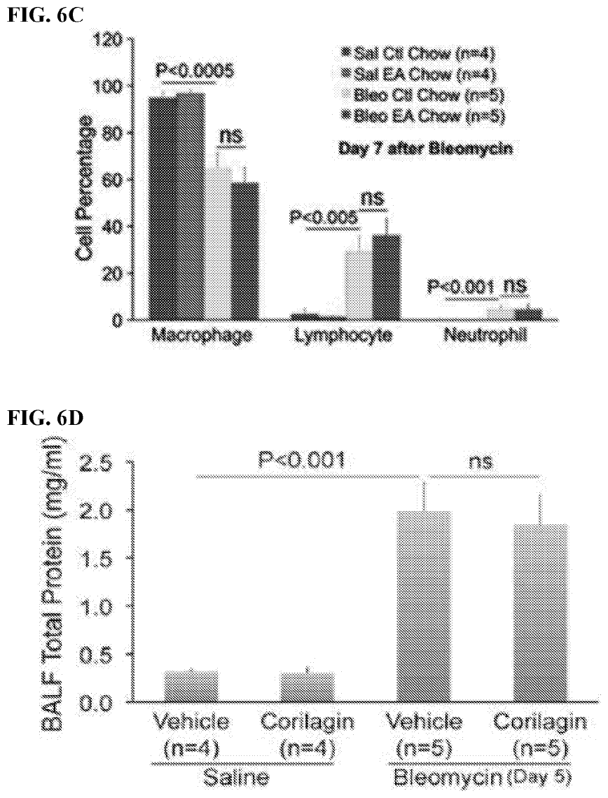

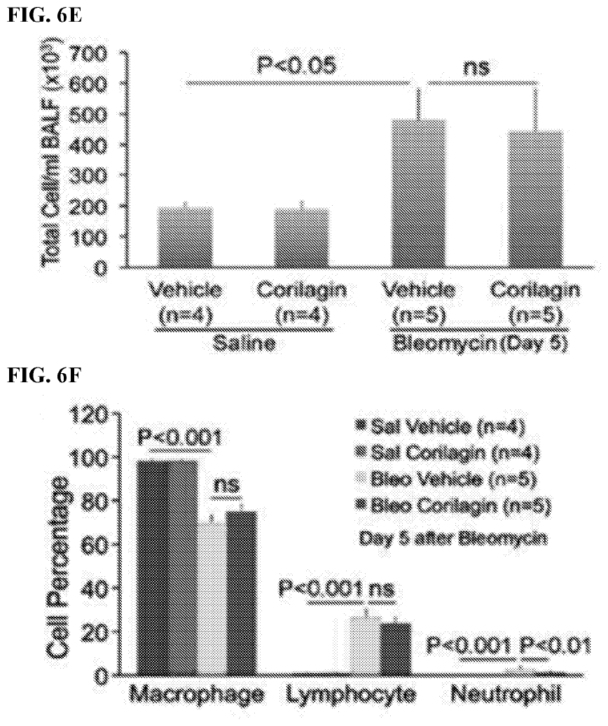

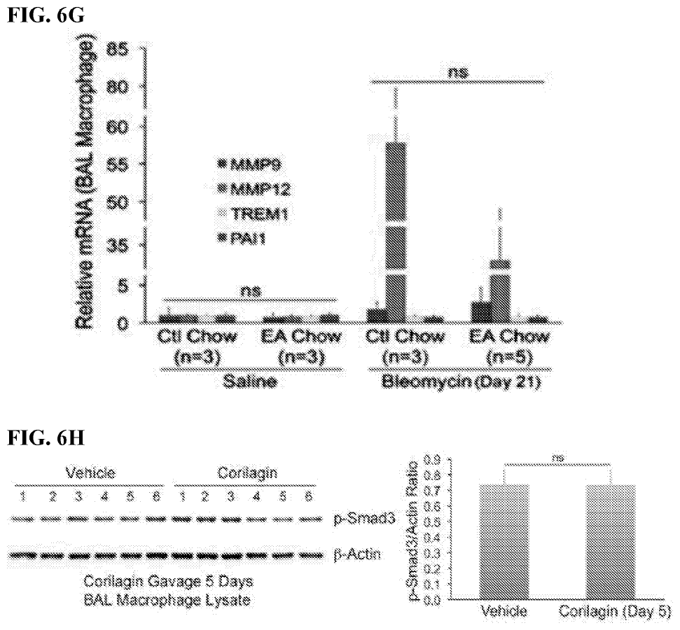

In an attempt to develop a more circumscribed inhibitor of TGFβ1 signaling centered on suppression of collagen accumulation a high-throughput, image-based phenotypic screen of small molecules that could block TGFβ1-induced epithelial-mesenchymal transition (EMT) in vitro but not immediately inhibit TGFβ1 receptor kinase itself was undertaken. We took advantage of the dramatic phenotypic switch in A549 adenocarcinoma cells upon TGFβ1 stimulation resulting in loss of E-cadherin expression and induction of fibronectin (FIG. 1A-1C)¹⁶. Several small molecule libraries totaling 40,000 compounds comprised of both diverse and bioactive compounds were screened. We identified ellagic acid (EA) as one compound meeting our criteria (FIG. 5A). EA was tested in vivo by ad libitum feeding of chow comprised of 2% w/w raspberry extract rich in EA and EA precursors given to mice after intratracheal bleomycin (FIG. 1J) and found to substantially block collagen accumulation (FIG. 1D, 1E) and improve survival (FIG. 5B). Because EA is poorly soluble, a more soluble trihydroxyphenolic compound, corilagin with IC50 for EMT 50 nM (FIG. 1F-1GF), was given daily by gavage beginning 10 days after intratracheal bleomycin (FIG. 1J). At day 21 these mice exhibited marked attenuation of bleomycin-induced total lung collagen, fibronectin, snail 1, and p-smad3 (FIG. 1H-1I). The average circulating level of corilagin 2 hrs after the last dose was 80 nM (FIG. 1C). EA-rich chow and corilagin had no effect on immune cell numbers or markers of injury (FIG. 6A-6H). To test the efficacy of trihydroxyphenolics in a second in vivo model¹⁷, we examined the occurrence of metastatic lung nodules in mice injected subcutaneously 5 weeks earlier with syngeneic Kras/p53 mutant lung tumor cells (344SQ) known to metastasize as a function of cross-linked fibrillar collagen content (FIG. 1K)¹⁷. Consumption of EA-rich chow following tumor implantation did not change primary tumor weight or size (FIG. 1L and FIG. 1D) but led to markedly reduced metastatic lung nodules (FIG. 1M). Accordingly we observed the primary tumor fibrillar collagen content to be reduced (FIG. 5E) and immunoblotting extracts of these tumors revealed attenuated total fibronectin, collagen I, and p-smad3 (FIG. 1N).

### Example 3. Identification of Trihydroxyphenolic Motif

We next examined the determinants of TGFβ1-induced EMT suppression in A549 cells by other polyphenol family members and found polyphenols with at least one trihydroxyphenolic motif in their primary structure inhibited snail 1 expression and the potency of inhibition correlated with the number of trihydroxyphenolic units (FIG. 5F-5H). This point is best illustrated by a comparison of epicatechin (EC) and epigallocatechin (EGC) which are structurally identical aside from a dihydroxy- rather then trihydroxy-phenolic motif in EC (FIG. 5H). EC had no activity in our in vitro assays whereas EGC was a potent inhibitor of snail 1 and fibronectin in TGFβ1 stimulated A549 cells. Collectively, these findings demonstrate that trihydroxyphenolic compounds attenuate TGFβ1-induced snail 1 and EMT markers in vitro as well as collagen accumulation in vivo and do so at low nM levels. We next turned to underlying mechanisms that could account for the activity and potency we observed.

### Example 4. LOXL2 Target Identification

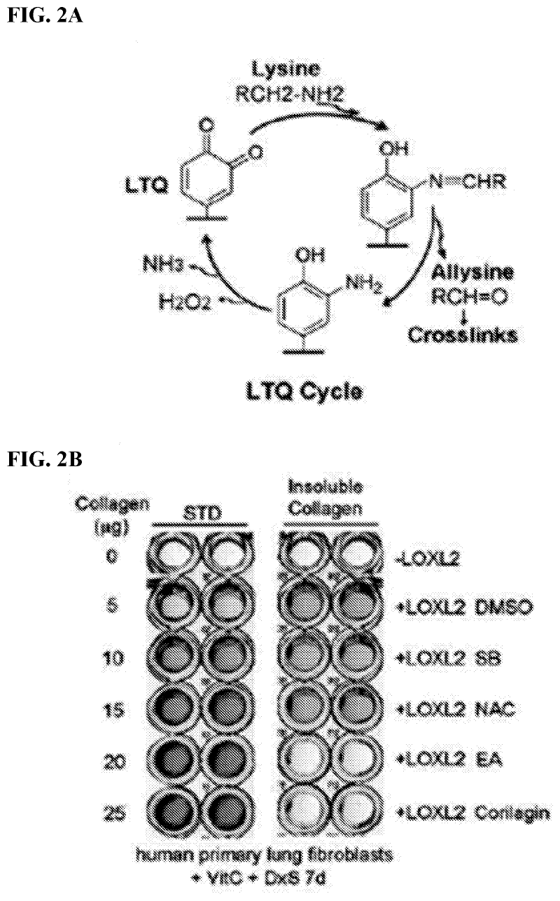

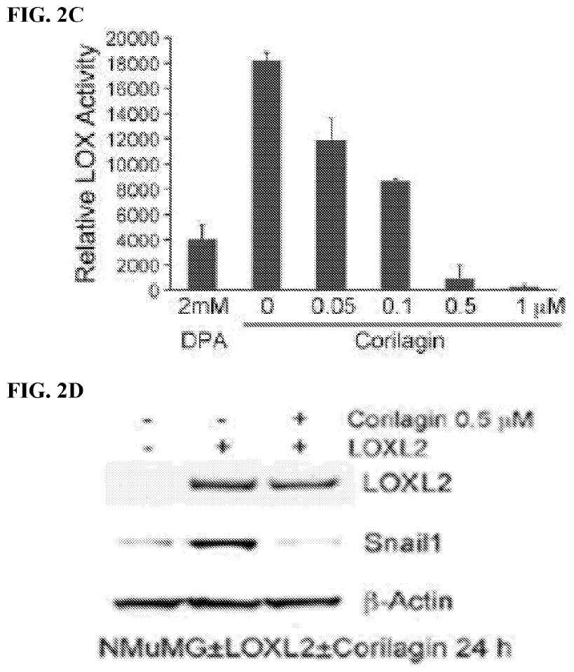

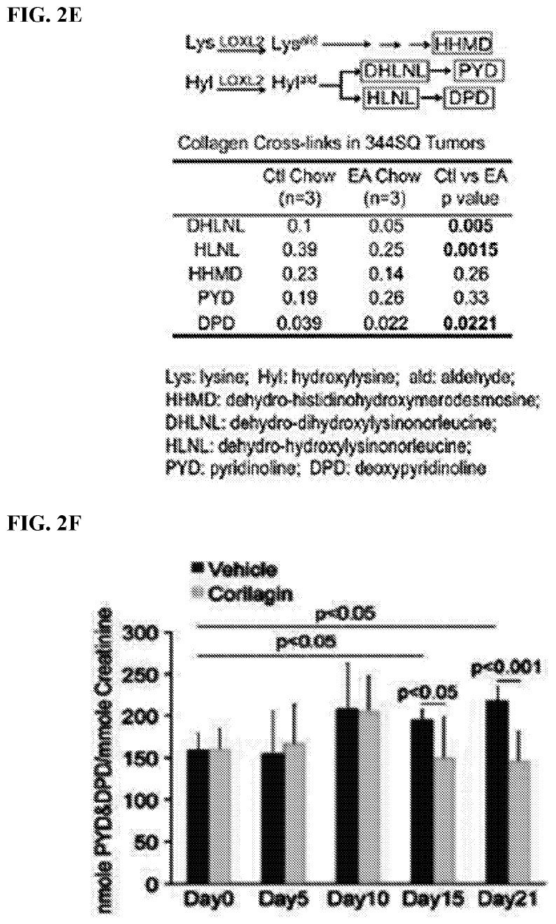

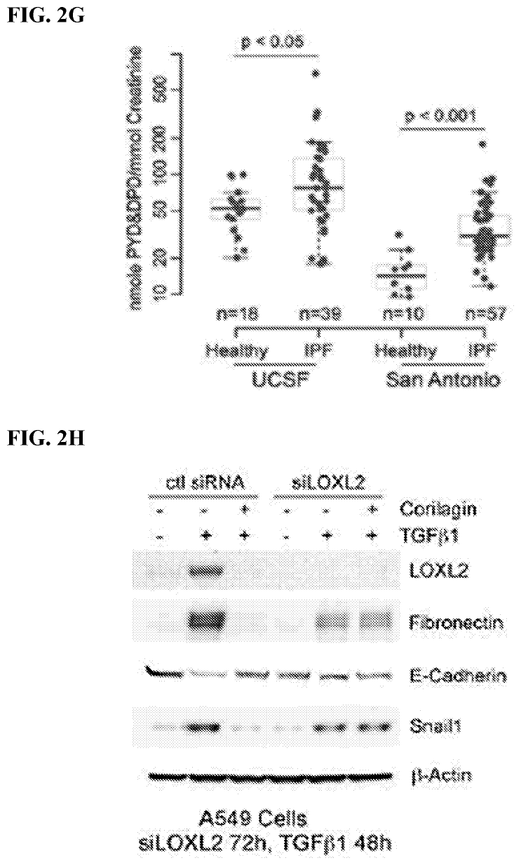

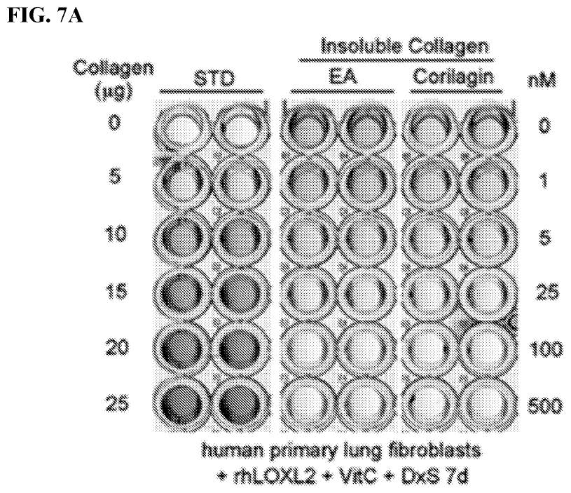

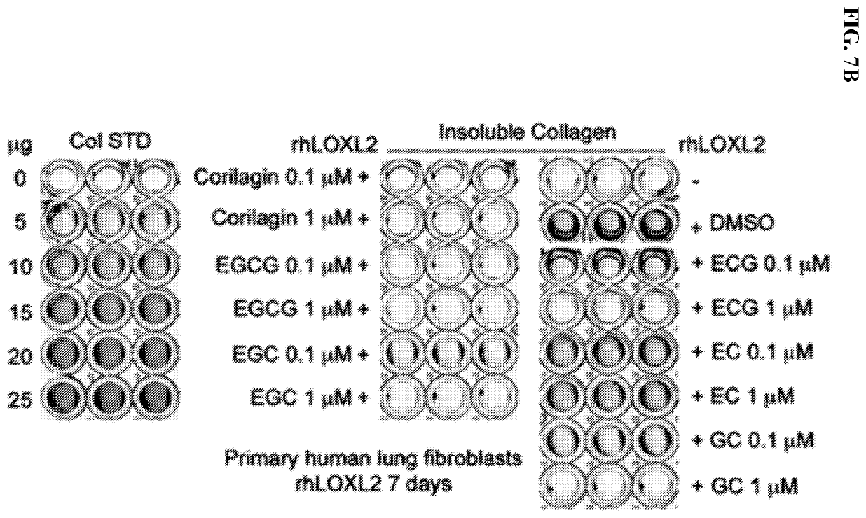

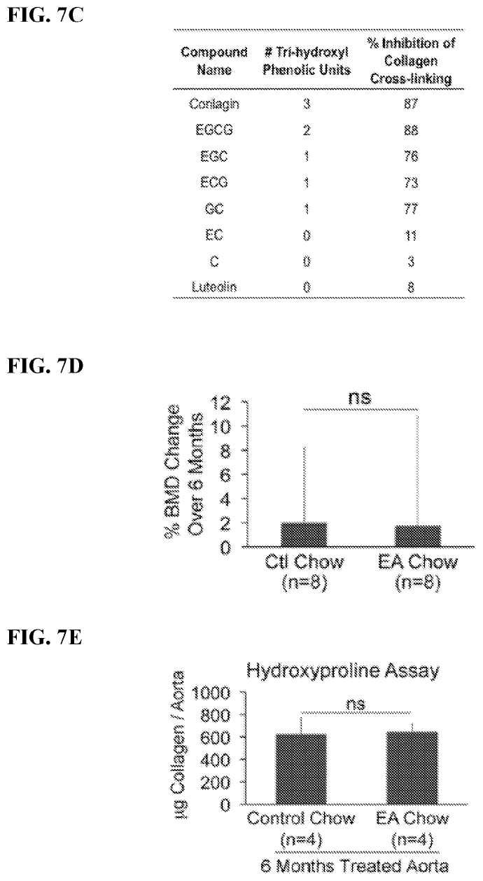

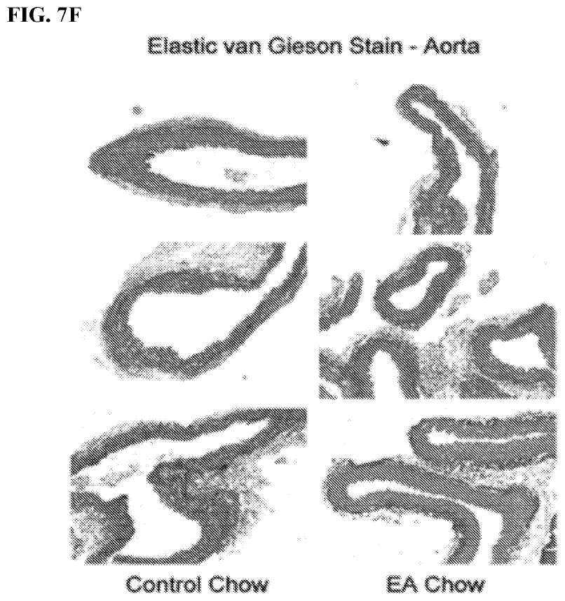

Because of the striking inhibition of snail 1 expression by several trihydroxyphenolic compounds, as well as the altered collagen crosslinking structure in primary 344SQ tumors (FIG. 5E), we explored the hypothesis that LOXL2 was a target of EA and corilagin. LOXL2 has previously been linked to snail 1 accumulation in tumor cells¹⁸ and is potently induced by both hypoxia and TGFβ1^{19,20}. LOXL2, like all mammalian copper-dependent LOX enzymes, utilize an intrinsically generated quinone termed LTQ to mediate oxidation of lysine primary amines^{3,4} (FIG. 2A). The catalytic site quinone oxidizes the primary amine releasing hydroxylysine (Hyl^{ald}) or lysine (Lys^{ald}) aldehyde, creating an intermediate aminophenol followed by release of H₂O₂ and NH₃ completing the LTQ cycle. Lys^{ald}/Hyl^{Ald} in monomeric collagens then undergo a series of spontaneous interactions that result in covalent crosslinks. We first established an assay of collagen crosslinking induced by recombinant LOXL2^{21,22} and found corilagin and all other trihydroxyphenolics tested to block crosslinking (IC50=10 nM, FIG. 7A-7C) whereas an inhibitor of TGFβ1 signaling (SB, SB431542) and an antioxidant (NAC, N-acetylcysteine) had no effect (FIG. 2B). LOXL2 enzymatic activity toward a model substrate was assessed by H₂O₂ release. Corilagin blocked activity with an IC50 of 50 nM (FIG. 2E). LOXL2-induced stabilization of snail 1 protein was also blocked by corilagin (FIG. 2D).

### Example 5. In Vivo Crosslinking Inhibition

To assess inhibition of LOXL2 activity in vivo we performed biochemical analyses of collagens extracted from primary 344SQ tumors of mice treated with EA or control chow^{23,24} (FIG. 1K). Accumulation of Hyl^{ald} derivatives including dehydro-hydroxylysineonorleucine (HLNL), dehydro-dihydroylysinonorleucine (DHLNL), and deoxypyridinoline (DPD) were all significantly decreased in primary tumors of mice fed EA chow, consistent with the altered collagen organization demonstrated by quantitative analysis of second harmonic generation from the primary tumors (FIG. 2E and FIG. 5E), and confirming inhibition of crosslinking activity by trihydroxyphenolics in vivo. Consistent with these findings urinary DPD/PYD levels were increased days 10-21 post bleomycin injection and this increase was suppressed by treatment of the mice with corilagin (FIG. 2F). Long-term EA chow treatment (6 months) did not affect mouse total bone mineral density or collagen content in the aorta (FIG. 7D-7F). Further, increased mean levels of PYD/DPD were observed in two cohorts of patients with Idiopathic Pulmonary Fibrosis (FIG. 2G), suggesting a signal from fibrotic lungs that could be used to track collagen turnover and drug response in vivo.

### Example 6. Compound Metabolism by LOXL2

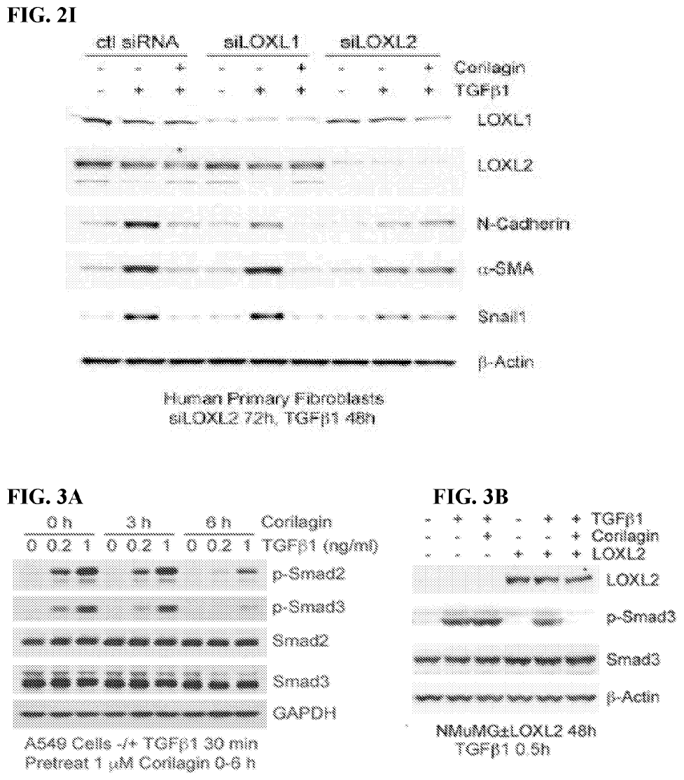

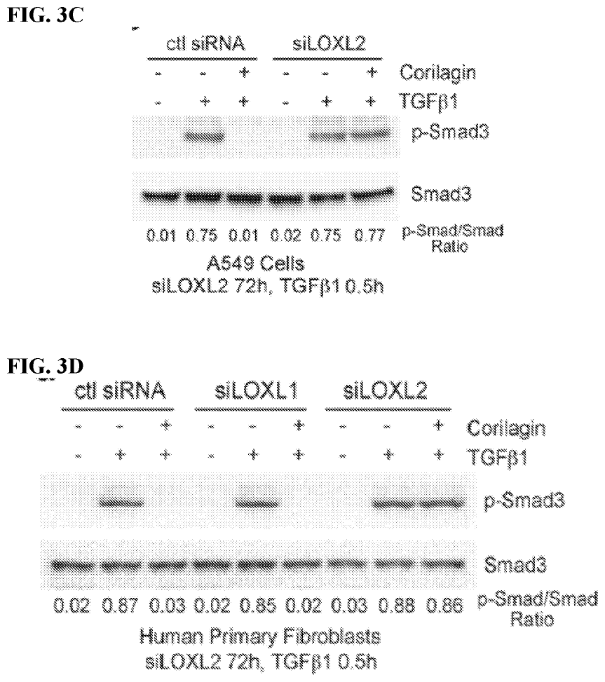

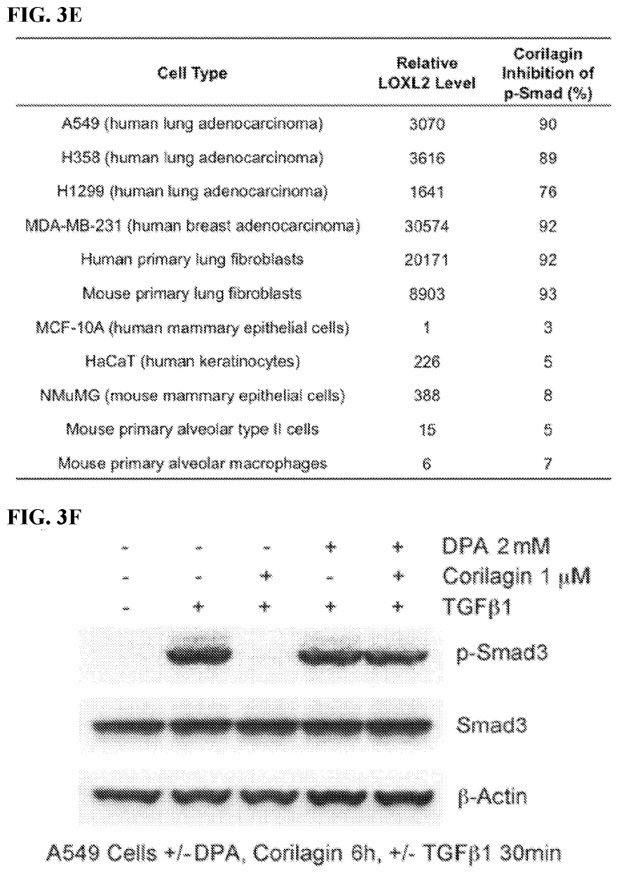

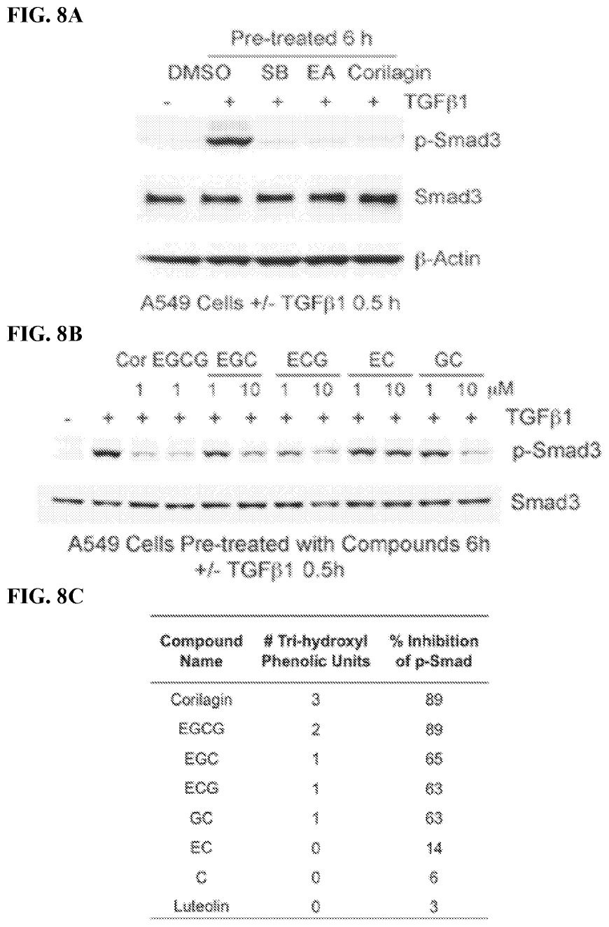

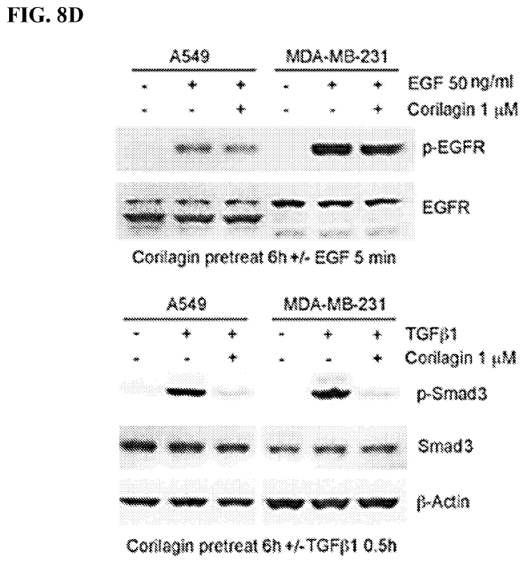

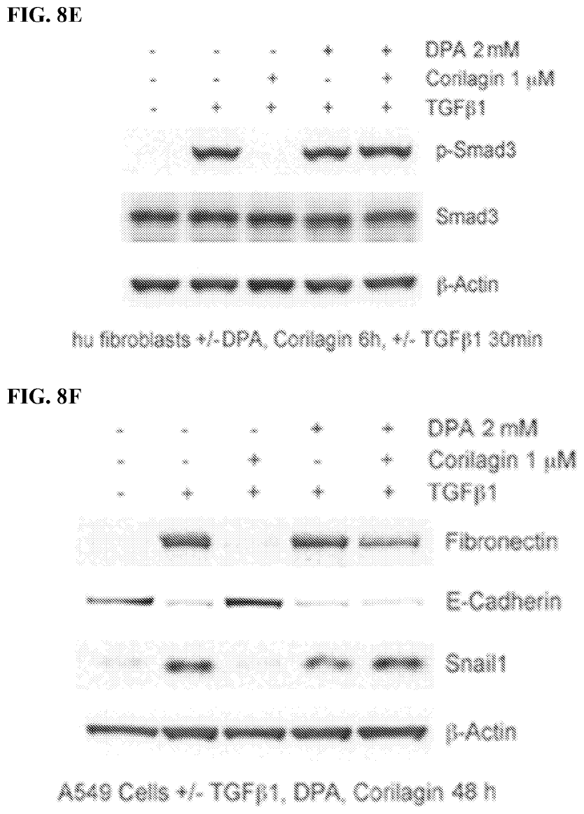

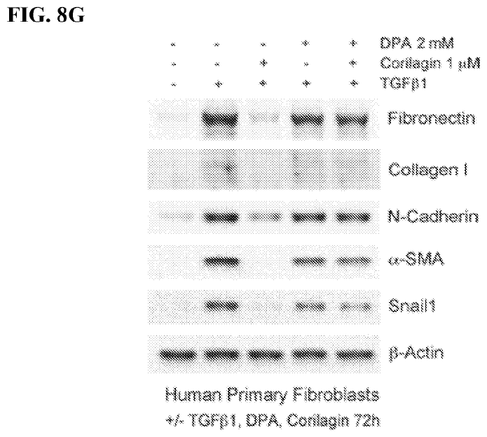

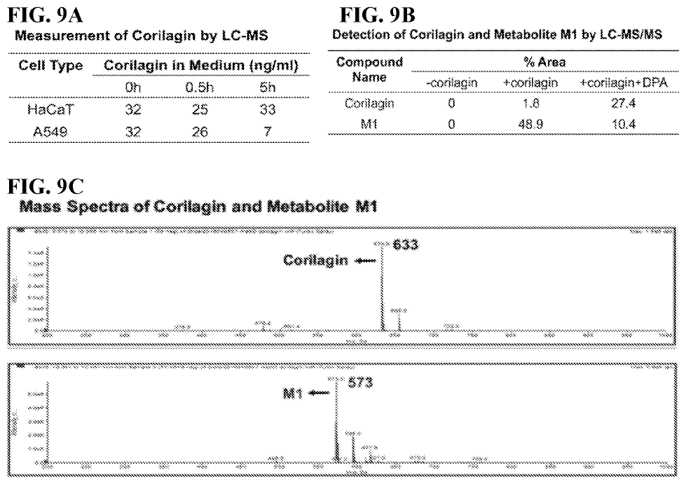

To further interrogate the impact of LOXL2 inhibition on TGFβ1 signaling we suppressed LOXL2 levels with RNAi in A549 cells. Surprisingly, rather than inhibiting TGFβ1, knockdown of LOXL2 completely abrogated the inhibitory effects of corilagin on EMT (FIG. 2H). Further, LOXL2 but not LOXL1 knockdown in fibroblasts completely prevented corilagin inhibitory effects on TGFβ1-induced mesenchymal proteins N-cadherin, α-SMA and snail 1 (FIG. 2I). These findings revealed that the mechanism of action was not simply LOXL2 inhibition, prompting us to revisit corilagin effects on TGFβ1 signaling. Although trihydroxyphenolic compounds did not block TGFβ1-induced p-smad generation in short-term assays (FIG. 1C), we observed that longer pre-incubation of cells with corilagin (or other trihydroxyphenolics) completely suppressed p-smads (FIG. 3A and FIG. 8A-8C) whereas in a similar design corilagin had no effect on EGF signaling through EGFR (FIG. 8D). Similarly, expression of LOXL2 in LOXL2-low NMuMG cells conferred corilagin responsiveness and blockage of p-smad3 following 6 hr pretreatment (FIG. 3B). Conversely, knockdown of LOXL2 in A549 cells and primary lung fibroblasts completely blocked corilagin effects on p-smad3 generation (FIG. 3C-3D). Consistent with a critical role for LOXL2 in proximal TGFβ1 signaling, a survey of numerous cell lines revealed a direct correlation of LOXL2 mRNA levels with the degree of inhibition of p-smad generation by corilagin (FIG. 3E). Finally, we confirmed that inhibition of active LOXL2 by the copper chelator penacillamine (DPA) also abrogated corilagin effects on smad signaling and snail 1 induction (FIG. 3F and FIG. 8E-8G). Together these data indicate that trihydroxyphenolic compounds selectively target proximal TGFβ1 signaling and snail 1 accumulation only in cells expressing LOXL2 and do so by a mechanism requiring the presence of active LOXL2. Analyses of LOXL2-dependent corilagin metabolism by LC-MS/MS suggested a nitrogen on a major corilagin metabolite (FIG. 9A-9C). These results raised the possibility that the trihydroxyphenolic motif operates as a LTQ-like mimic leading to its metabolism by LOXL2 and generating an inhibitor of TGFβ1 signaling.

### Example 7. Identification of LOXL2 Reactive Lysine K731

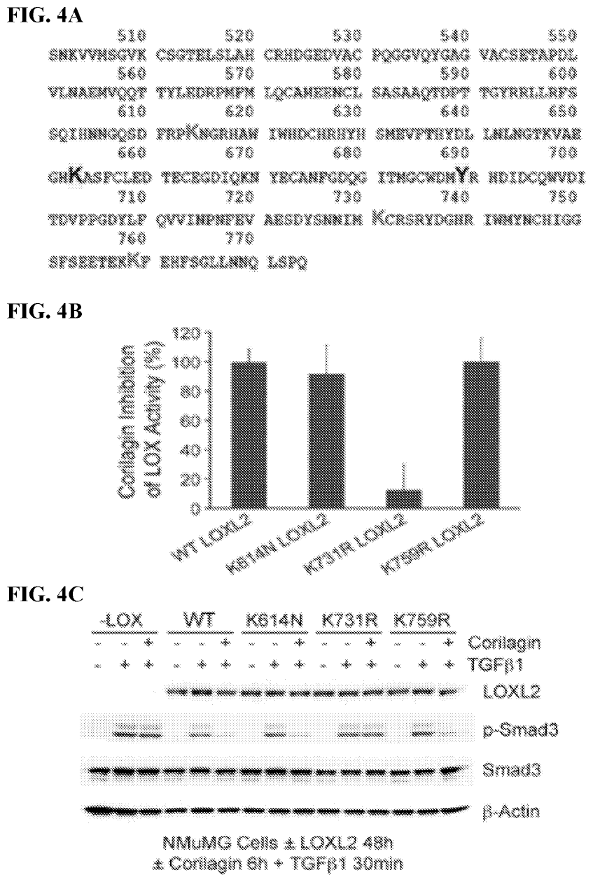

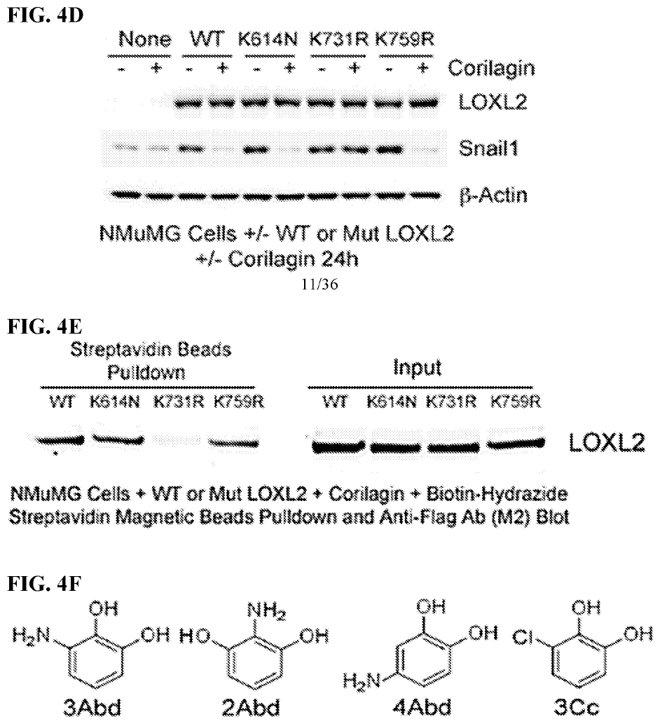

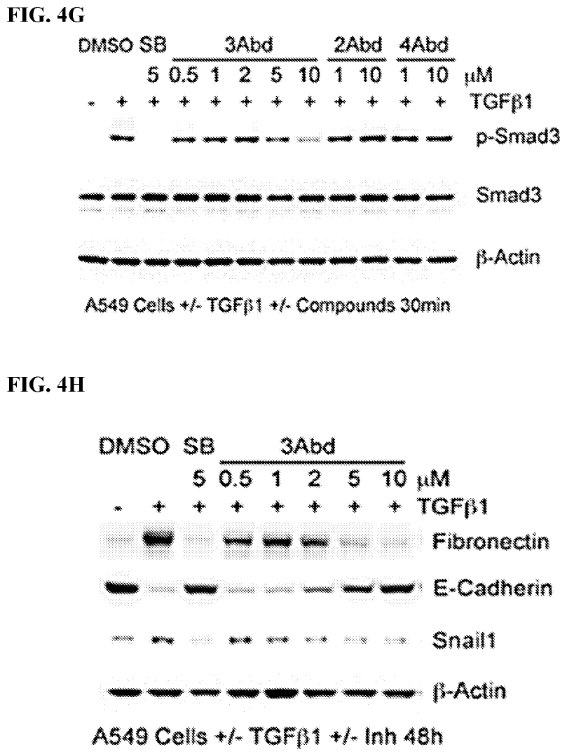

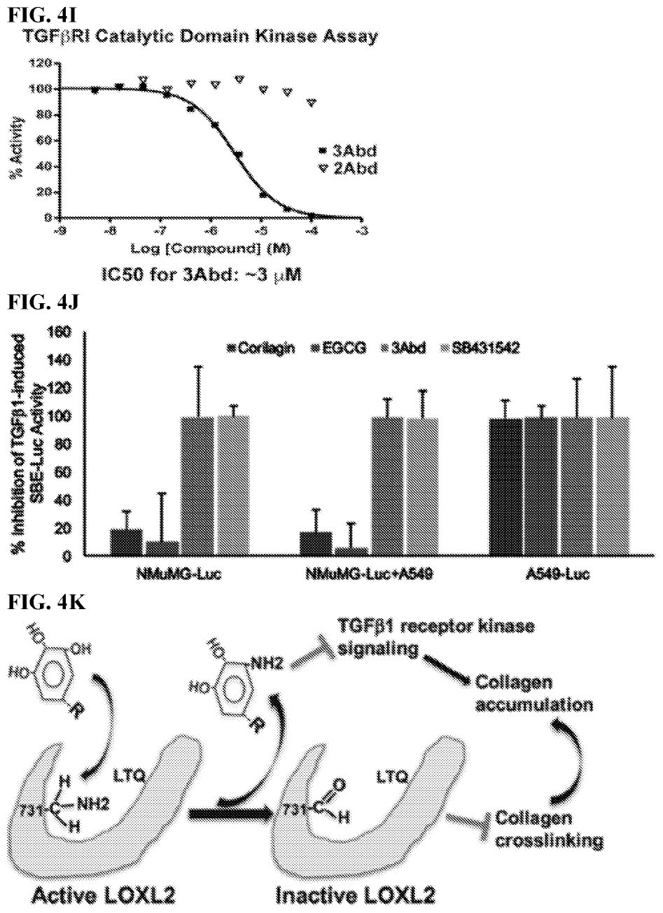

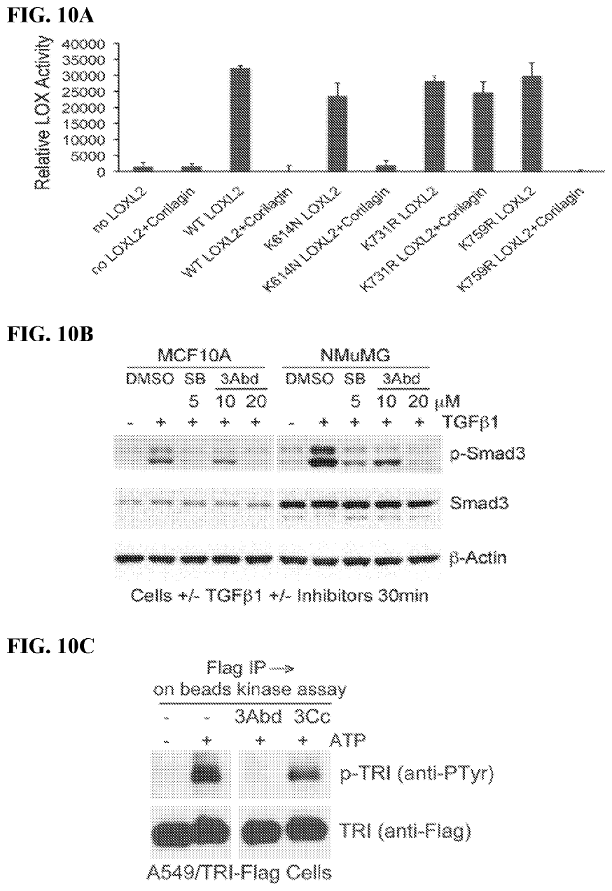

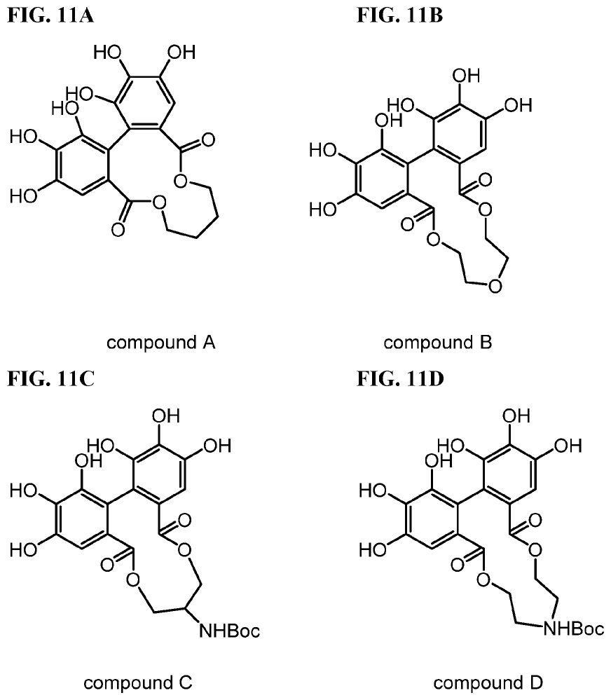

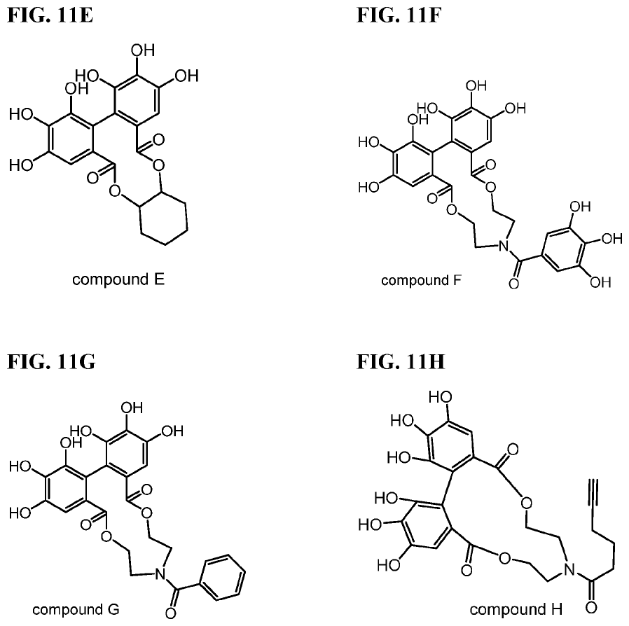

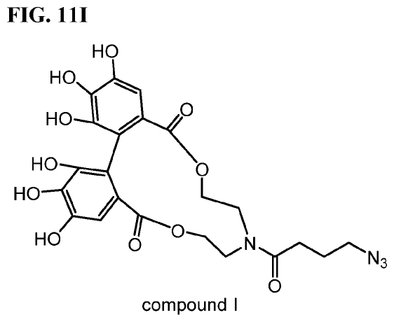

To begin to test this hypothesis we inspected the LOX catalytic domain lysines specific to LOXL2 over LOX/LOXL1 (FIG. 4A), whose knockdown had no effect (FIG. 2I, FIG. 3D). We then established and expressed flag-tagged point mutants of each of the three lysines specific to LOXL2/3, converting to the corresponding LOXL1 residues: K614N, K731R, and K759R. Each of the point mutants had comparable enzyme activity to wt LOXL2 when expressed in NMuMG cells (FIG. 10A) and all except the K731 mutant were completely inhibited by corilagin (FIG. 4B). Cells expressing K731R LOXL2 were completely resistant to inhibition of TGFβ1 signaling by corilagin whereas the other mutants were indistinguishable from wt (FIG. 4C-4D). Because lysine auto-oxidation by LOX family enzymes is critical to LTQ generation we asked whether K731 was auto-oxidized to an aldehyde in the presence of corilagin. Incubation of a biotin-hydrazide that covalently links to free aldehydes with immunoprecipitated wt and mutant LOXL2 confirmed that each enzyme except the K731R mutant developed an aldehyde when mixed with corilagin, implying K731 but not other lysines is converted to an aldehyde during metabolism of corilagin by LOXL2 (FIG. 4E).

### Example 8. Identification of Reactive Hydroxyl

We next screened compounds structurally similar to the intermediate aminophenol known to appear during the LTQ cycle (FIG. 2A) for direct TGFβ1 inhibition. A catechol containing an amino group at position 3 (3Abd, 3-aminobenzene-1,2-diol) but not at either position 2 (2Abd) or 4 (4Abd) was found to be a potent inhibitor of TGFβ1-induced p-smad3 and snail 1 without pre-incubation and regardless of LOXL2 expression (FIGS. 4F-4H and FIG. 10B). We confirmed that 3Abd, but not 2Abd, directly blocked the kinase activity of recombinant TGFβRI catalytic domain with an IC50 3 μM, consistent with the inhibitory profile of 3Abd in cells (FIG. 4H). In cells overexpressing TGFβRI we also observed that 3 Abd but not control 3 Cc blocked activity in a pull down kinase assay (FIG. 10C). Of note, 3 Abd is structurally distinct from any of the known low molecular weight inhibitors of TGFβRI².

### Example 9. Intracellular Activity of Identified Compounds

To further define the mechanism of TGFβ1 inhibition by trihydroxyphenolic compounds we asked whether secreted LOXL2 generated a 3Abd-like metabolite. Although LOXL2-mediated metabolites of corilagin could be detected (FIG. 9A-9C), no inhibitory activity was observed in conditioned media and the structure of these metabolites were not pursued further. Overnight co-culture of corilagin-treated A549 cells with corilagin-nonresponsive NMuMG cells (FIG. 3B) expressing a smad binding element reporter (12X CAGA)²⁵ revealed indistinguishable TGF pi-induced reporter activation with or without 5-fold excess A549 cells in co-culture, indicating a diffusible inhibitor was not detectable (FIG. 4J). Together these studies point to an intracellular origin of a trihydroxyphenolic metabolite(s) inhibiting TGFβ1 receptor kinase (FIG. 4K). While the predicted LTQ-like intermediate, 3 Abd, acts as a direct TGFβRI kinase inhibitor (FIG. 4H), future studies will be needed to isolate the exact inhibitory metabolite(s) present within trihydroxyphenol-treated LOXL2 expressing cells. Nonetheless, these studies elucidate a novel approach to target pathologic collagen accumulation by potently blocking TGFβ1 signaling specifically in fibroblast-like cells, avoiding the likely toxicities of global TGFβ1 inhibition for chronic disease processes such as fibrosis and cancer progression.

### Example 10. Experimental Methods and Materials

Reagents. Ellagic acid, epigallocatechin gallate, epicatechin gallate, epigallocatechin, gallocatechin, epicatechin, catechin, luteolin, chloramine T, p-dimethylaminobenzaldehyde, bleomycin, D-penicilamine, N-acetylcysteine, pyrogallol, protease and phosphatase inhibitor cocktails, flbronectin pAb, α-SMA mAb, Flag M2 mAb, and β-actin mAb were purchased from Sigma-Aldrich (St Louis, Mo.). Corilagin was purchased from BOC Sciences (New York, N.Y.). TGFβ type I receptor inhibitor SB431542 and phospho-smad2 (Ser 465/467) pAb were from EMD Millipore (Billerica, Mass.). Snail1 mAb, smad2 mAb, phospho-smad1/5 (Ser463/465) pAb, smad1 pAb, phospho-EGFR pAb, and EGFR pAb were from Cell Signaling (Beverly, Mass.). Collagen I pAb, vimentin mAb, LOXL2 pAb for Western blot, and phospho-smad3 (Ser 423/425) pAb were from Abeam (Cambridge, Mass.), siRNAs for human LOXL1 and LOXL2, and secondary HRP-conjugated antibodies were from Santa Cruz Biotechnology (Santa Cruz, Calif.). Streptavidin-magnetic beads, TurboFect transfection reagent, and EZ-Link Hydrazide-LC-Biotin were from Thermo Scientific (Waltham, Mass.). E-cadherin and N-cadherin antibodies were from BD Biosciences (San Jose, Calif.). TGFβ1 was from PeproTech. Alzet osmotic pump 1007D was purchased from DURECT Corporation (Cupertino, Calif.). Red raspberry diet and red control diet was custom made by Envigo (Indianapolis, Ind.) Sircol Insoluble Collagen Assay kit was from Biocolor (Westbury, N.Y.). MicroVue PYD EIA Kit and MicroVue Creatinine EIA Kit were purchased from Quidel Corporation (San Diego, Calif.). Wild-type LOXL2 (pcDNA3-hLOXL2-flag) plasmid was a gift. 3-aminobenzene-1,2-diol are from AURUM Pharmatech Inc. (Franklin Park, N.J.). 2-aminobenzene-1,3-diol, 4-aminobenzene-1,2-diol, and 3-chlorocatechol were from Astatech Inc. (Bristol, Pa.).

Cell culture. Human or mouse cell lines were purchased from ATCC (Manassas, Va., USA) and grown in DMEM or RPMI1640 medium supplemented with L-glutamine and 10% FBS (Hyclone, Logan, Utah, USA). Human and mouse lung fibroblasts were isolated from crude whole lung single-cell suspension cultures on petri dish in Dulbecco's Modified Eagle Medium supplemented with L-glutamine and 10% FBS for 2 weeks. Mouse type II alveolar epithelial cells (AECs) isolation and culture was performed as previously described³².

High throughput screen and high content imaging analysis. A549 cell-based screening of inhibitors to TGFβ1-induced EMT from a bioactive small molecule library was performed in 384-well plate format and the images were captured and analyzed using GE IN Cell 2000 as described previously¹⁶.

Immunofluorescence. Cultured cells and 5-7 μm cryosections were fixed in 4% paraformaldehyde and stained with various antibodies and IgG isotype controls. Where indicated in the figure legends, mosaic images were generated from multiple ×20 images captured on a Zeiss Axio upright fluorescent microscope and tiled using 10% image overlap by Axiovision 4.7 software.

Masson's Trichrome stain. For histological assessment of lung collagen, frozen sections of the left lung were stained using Masson's Trichrome stain kit (American MasterTech, Lodi, Calif., USA). The whole section was imaged with a Zeiss Axio upright microscope and tiled using 10% image overlap into a single panoramic by Axiovision 4.7 software (Zeiss).

Immunoblot. Pulverized tissue and cells were lysed in RIP A buffer (150 mM NaCl, 50 mM Tris, pH 8.0, 1% Triton X-100, 0.5% sodium deoxycholate, 0.1% SDS, supplemented with protease and phosphatase inhibitors) and analyzed by immunoblotting. Densitometry was quantified using NIH Imaged software.

Bleomycin fibrosis model. Eight-week old C57BL/6 mice were intratracheally instilled with saline or 1.9 units/kg of bleomycin (Sigma-Aldrich). Mice were implanted with osmotic pumps loaded with ellagic acid salt (24 mg/kg/day, Day 10-Day 17), fed with red raspberry diet (Day 0-Day 21), or gavaged with corilagin (100 mg/kg, Day 10-Day 21). Controls were treated with control pump, control diet, or vehicle in the same formulation. The lungs were lavaged and followed by OCT embedding for imaging or snap freezing in liquid nitrogen for protein extraction or hydroxyproline assay.

Syngeneic in vivo tumorigenesis and metastasis assays. KrasG12D/p53R172H metastatic lung cancer cells (344SQ) were subcutaneously injected in the right flanks of male, syngeneic 129/sv mice at 3 months of age and allowed to form tumors for 5 to 6 weeks. The mice were fed with red raspberry diet or control diet. After euthanasia, tumors were measured and lung metastatic nodules were quantified. Primary tumor tissues were snap frozen and analyzed by Western blot. Some primary tumors were formalin fixed, paraffin embedded, and sectioned for second harmonic generation imaging. All animal experiments were reviewed and approved.

Immunohistochemistry (IHC) and second harmonics generation (SHG) microscopy. Paraffin embedded tissue sections were rehydrated, blocked with goat serum, and probed for collagen I. Tissues were subsequently washed and probed with HRP-conjugated secondary antibodies and signal was attained by developing with a DAB reagent. For SHG microscopy, tissues stained by H&E were visualized using a Zeiss LSM 7 MP Multi photon Microscope at an excitation wavelength of 800 nm and collagen fiber signals were detected at 380-430 nm using bandpass filters. Collagen linearity was calculated as a ratio of the total length versus the end to-end length of the individual collagen fiber.

Collagen content Lung or aorta collagen content was evaluated using hydroxyproline assay. Briefly, whole left lung tissue or aorta was hydrolyzed in 1 ml 12N HCl at 110° C. for 24 h and the hydroxyproline was detected by incubating with Chloramine T and p-dimethylaminobenzaldehyde and the absorbance was measured at 550 nm. Each sample was run in triplicate. Collagen content in lung or aorta tissues was expressed as micrograms of collagen per lung or aorta and was converted from micrograms of hydroxyproline.

Bronchoalveolar lavage. After the trachea was exposed, a 20-G catheter was inserted into the trachea through a small incision. 1 ml cold PBS was instilled into the mouse lungs followed by gentle aspiration repeated for three times. All the bronchoalveolar lavage fluid (BALF) was centrifuged and cell pellet was re-suspended in erythrocyte (RBC) lysis buffer (Sigma, St. Louis) followed by re-centrifugation. Cell number was counted using hemocytometer. Cell types of BALF were determined by morphology following Diff-quick stain of cytospin slides. About 500 cells were counted for each sample in order to determine the cell types. Macrophages account for more than 80% of the cells in BALF and were collected by centrifugation. After centrifugation, the supernatant was collected to measure total protein content using the BCA assay (Pierce), while the cell pellet was lysed for immunoblotting or RNA isolation.

Plasma level of corilagin—LC/MS analysis. Corilagin in C57BL/6 mice two hours following last oral administration at day 21 was analyzed. Blood samples (˜500 μL/sample) were collected via cardiac puncture. Samples were placed in tubes containing heparin sodium and stored on ice until centrifuged for plasma.

Preparation of insoluble cross-linked collagen. Fibroblasts were cultured on 10 cm dish until confluent. The medium was then changed to Dulbecco's modified Eagle's medium (DMEM) containing 5% FBS, 100 μM L-ascorbic acid with 500 kDa Dextran Sulfate at 100 μg/ml, and 50 ng/ml recombinant human LOXL2 (rhLOXL2) for 7 days^{21,22}. The cell layer was extracted with 0.5 M acetic acid and 0.1 mg/ml pepsin overnight at 4° C. The leftover insoluble fraction was further extracted and the insoluble cross-linked collagen measured using Sircol Insoluble Collagen Assay kit according to manufacture's instruction.

LOX activity assay LOX activity of recombinant human LOXL2 or conditioned medium collected from cells expressing LOXL2 was measured using a Fluorimetric Lysyl Oxidase Activity Assay Kit (Abeam, Cambridge, Mass.) following a protocol provided by the manufacturer. Briefly, 50 μl of sample was mixed with an equal volume of assay reaction mixture containing LOX substrate, horseradish peroxidase (HRP) and HRP substrate in the presence and absence of testing inhibitors and D-penicillamine (DPA), a LOX inhibitor. The mixture was incubated for 30 min at 37° C. in darkness. The fluorescence increase was then measured with a fluorescence plate reader (BMG Lab Tech FLUOstar) at Ex/Em=540/590 nm. Sample buffer or medium alone without LOXL2 was used for determination of the background fluorescence.

Collagen crosslink analysis. Snap frozen primary 344SQ tumors were pulverized in liquid nitrogen using a Spex Freezer Mill (Spex, Metuchen, N.J.), washed with cold PBS and cold distilled water, lyophilized, and weighed. Aliquots were reduced with standardized NaB3H4 and hydrolyzed with 6N HCl. The hydrolysates were then subjected to amino acid and cross-linking analyses using LC-MS/MS as described previously³³. The terms DHLNL, HLNL, and HHMD represent both the unreduced and reduced forms. The mature trivalent cross-links, PYD and DPD, were simultaneously analyzed by their fluorescence. All cross-links were quantified as the mol/mol collagen based on the value of 300 residues of hydroxyproline per collagen molecule.

Urinary PYD/DPD measurements. Pooled urine from each of 3-5 mice for each time point after bleomycin in a cohort of mice treated with vehicle or corilagin (100 mg/kg) beginning on day 10 post bleomycin were collected. Urine specimens were also collected from two cohorts of IPF patients and controls at two sites: All consenting patients with physician-established diagnosis of IPF followed in the respective ILD programs were included in sample collection. PYD/DPD levels from all the samples were measured using MicroVue EIA Assay Kit (Quidel Corp., San Diego, Calif.) along with PYD/DPD standards and the results were normalized relative to urinary creatinine. The statistical significance was analyzed using Mann-Whitney U-Test.

Bone mineral density (BMD) measurement. BMD of mice treated with red raspberry diet or control diet up to 6 months was measured using Dual-energy X-ray absorptiometry (DEXA) scan. DEXA scans were performed using the Lunar PIXImus Densitometer (GE Medical Systems, Waukesha, Wis.) at UCSF animal facility. PIXImus Densitometer was calibrated before each testing using a quality control phantom following the manufactures' instructions.

Elastic van Gieson stain. Aortas isolated from mice treated with red raspberry diet or control diet up to 6 months were embedded in paraffin (n=3 per group). Sections (5 [symbol] m) were cut every 30 [symbol] m along the aortas (starting from the proximal end). Selected sections were stained with Miller's Elastica van Gieson stain.

Site-directed mutagenesis of LOXL2. Site-directed mutagenesis was performed to generate K614N, K731R, and K759R point mutations using Phusion Site-Directed Mutagenesis Kit (Thermo Scientific, Waltham, Mass.) according to manufacturer's instructions. A pcDNA3-hLOXL2-flag plasmid containing the cDNA fragment of wild-type human LOXL2 fused with a flag tag at the C-terminus, was used as the template DNA¹⁸. Mutations were confirmed by DNA sequencing. The primers and their complementary strands used are: K614N forward 5′-GACTTCCGGCCTAATAATGGCCGC-3′ (SEQ ID NO:2), K614N reverse 5′-GGACTGGCCATTGTTGTGGATCTG-3′(SEQ ID NO:3); K731R forward 5′-ACAACATCATACGATGCAGGAGCC-3′(SEQ ID NO:4), K731R reverse 5′-TGGAGTAATCGGATTCTGCAACCT-3′(SEQ ID NO:5); K759R forward 5′-ACGGAAAAACGTTTTGAGCACTTCA-3′(SEQ ID NO:6), and K759R reverse 5′-CTCTTCGCTGAAGGAACCACCTAT-3′(SEQ ID NO:7).

Biotin hydrazide derivatization of carbonylated LOXL2. NMuMG cells were transiently transfected with wild-type or mutant human LOXL2-Flag in 10 cm dish and 24 hours later the cells were treated with 1 μM corilagin for 6 hours at 37° C. before lysis in 50 mMHEPES, 100 mM NaCl, 2 mMEDTA, 0.5% Triton-100 plus protease inhibitor cocktail, 10 mM NaF, 1 mM Na3 VO4. The lysates were incubated with 2.5 mM EZ-Link Hydrazide-LC-Biotin (Thermo Fisher) in dark for 2 h at room temperature. Biotin hydrazide bound proteins were captured using streptavidin-magnetic beads (Pierce) on a rotary mixer at 4° C. overnight. The beads were washed three times with lysis buffer and eluted with sample buffer for 10 min at 70° C. Biotin hydrazide linked carbonylated LOXL2 and total input LOXL2 were detected by LOXL2 polyclonal antibody (Abeam) blot.

In vitro TGFβ receptor kinase assay. A549 cells were transiently co-transfected with Flag-tagged human TGFbeta receptor I and II. After 24 hours the cells were lysed in 1% NP40 lysis buffer (1% NP40, 20 mM Tris pH7.6, 200 mM NaCl plus protease inhibitor cocktail, 10 mM NaF, 1 mM Na3VO4), and the type I and II receptors were immunoprecipitated using anti-Flag antibody and Protein G-agarose (Roche). The beads were washed three times with kinase buffer (0.01% Triton X-100, 25 mM HEPES, pH 7.4, 2 mM MnCl2, 10 mM MgCl2, 20 μM DTT, 0.1 mM NaF, 0.1 mM Na3 VO4). The kinase reactions were initiated by addition of 0.1 mM ATP in the presence or absence of inhibitors or lysate from A549 cells pre-treated with corilagin. The kinase reactions were terminated by addition of an equal volume of 2×sample buffer. The TGFbeta receptor kinase activity was analyzed by SDS-PAGE and immunoblotting with anti-phosphotyrosine monoclonal antibody 4G10 (EMD Millipore) and anti-Flag antibody.

ALK5/TGFβRI catalytic domain kinase assay. ALK5/TGFβRI kinase assays using purified catalytic domain was performed. The reaction buffer contains 20 mM Hepes (pH 7.5), 10 mM MgCl₂, 1 mM EGTA, 0.02% Brij35, 0.02 mg/ml BSA, 0.1 mM Na₃VO₄, 2 mM DTT, and 200 μM NAC. In brief, ten 3-fold series dilutions of 3Abd and 2Abd starting at 100 μM was delivered into kinase reaction mixture with kinase, cofactors, and substrate. After 20 min incubation at room temperature, ³³P-ATP was delivered into the mixture to initiate the reaction. Kinase activity was detected 2 h later by P81 filter-binding method.

Co-culture SBE reporter. NMuMG and A549 cells were transiently transfected with pGL(CAGA)₁₂Luc by using Turbofect reagent as specified by the manufacturer (Thermo Fisher). The transfected cells were seeded into 96-well plate in triplicates 24 hours after transfection. Co-cultured wells were seeded with transfected NMuMG cells and non-transfected A549 cells. The cells were pretreated with or without 1 μM corilagin, 1 μM EGCG, 10 μM 3Abd, or 5 μM SB431542 for 6 hours before stimulating with TGFβ1 overnight. The cells were lysed and luciferase activity was measured using the luciferase assay kit from Promega.

qRT-PCR analysis. Total RNA (1 μg of each sample isolated using RNeasy, Qiagen) was reverse transcribed using Superscript III (Invitrogen) and assayed for gene expression using Platinum Quantitative PCR SuperMix-UDG (Invitrogen). β-actin, GAPDH and S9 were used as internal controls and all the data were normalized by β-actin. The primer and probe sequences are listed in the Table below.

| Name | Sequence |
|---|---|
| Mouse MIMP9 F | 5′-TTCCTGGTGGCAGCGC-3′ |
| | (SEQ ID NO: 8) |
| Mouse MIMP9 R | 5′-CGGCACGCTGGAATGATC-3′ |
| | (SEQ ID NO: 9) |
| Mouse MIMP9 | 5′-/FAM/ |
| Probe | CCCAGTGCATGGCCGAACTCG/BHQ/-3′ |
| | (SEQ ID NO: 10) |
| Mouse MIMP12 F | 5′-TGTGGAGTGCCCGATGTACA-3′ |
| | (SEQ ID NO: 11) |
| Mouse MIMP12 R | 5′-AGTGAGGTACCGCTTCATCCAT-3′ |
| | (SEQ ID NO: 12) |
| Mouse MIMP12 | 5′-/FAM/ |
| Probe | CATCTTAGAGCAGTGCCCCAGAGGTCAA/ |
| | BHQ/-3′ |
| | (SEQ ID NO: 13) |
| Mouse PAI1 F | 5′-TGCATCGCCTGCCATTG-3′ |
| | (SEQ ID NO: 14) |
| Mouse PAI1 R | 5′-GACATTTCCACAGTGGACCTTGA-3′ |
| | (SEQ ID NO: 15) |
| Mouse PAI1 | 5′-/FAM/ |
| Probe | TTGGCCCATGGCACCCTCCA/BHQ/-3′ |
| | (SEQ ID NO: 16) |
| Mouse Trem1 F | 5′-CAAACCGCATCACCACAAAG-3′ |
| | (SEQ ID NO: 17) |
| Mouse Trem1 R | 5′-ACGTGGTTCAGCCACTCCTC-3′ |
| | (SEQ ID NO: 18) |
| Mouse Trem1 | 5′-/FAM/ |
| Probe | CACTTGATCGCACCCAGAAGGCC/BHQ/-3′ |
| | (SEQ ID NO: 19) |

Statistics. Variance for all group data is expressed as ±SEM. For evaluation of group differences, the unpaired 2-tailed Student's t-test was used assuming equal variance. A P value less than 0.05 was accepted as significant.

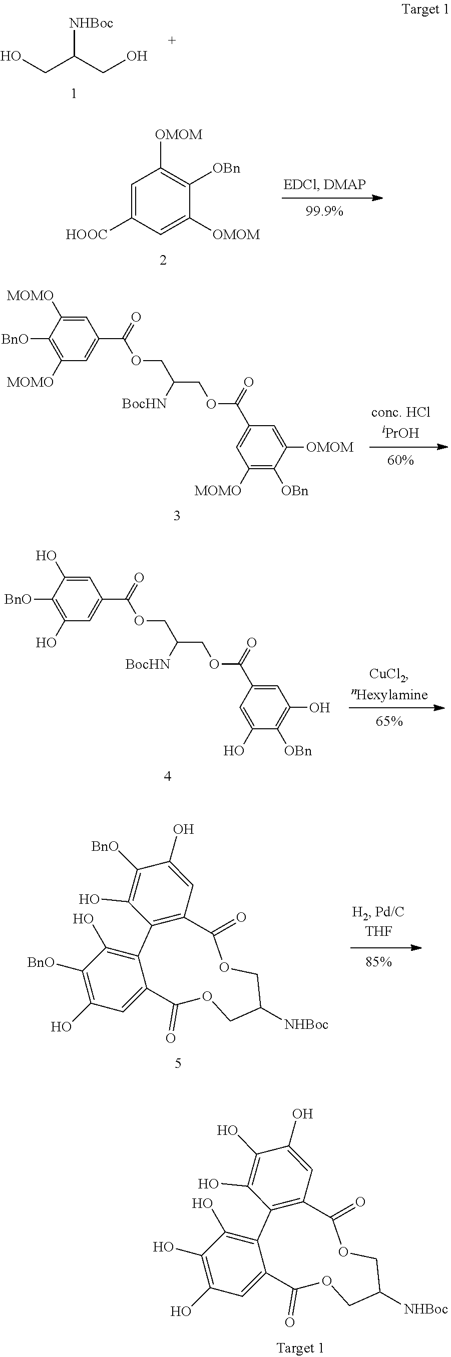

2-(tert-butoxycarbonylamino)propane-1,3-diyl bis(4-(benzyloxy)-3,5-bis(methoxymethoxy)benzoate) (3). Compound 2 (200 mg, 0.57 mmol) was dissolved in DCM (5 mL). EDCI (220 mg, 1.15 mmol), DMAP (190 mg, 1.61 mmol) and Compound 1(44 mg, 0.23 mmol) were added in sequence. The mixture was stirred at R.T. overnight. TLC showed the end of reaction. 1M H₃PO₄ was used to quench the reaction. The inorganic phase was extracted with DCM(X2). All the organic phases were combined and washed by 1M H₃PO₄, Sat. NaHCO₃, brine and dried over Na₂SO₄. Purification through flash column chromatography gave 3 (190 mg, 99.9%) as colorless oil.

2-(tert-butoxycarbonylamino)propane-1,3-diyl bis(4-(benzyloxy)-3,5-dihydroxybenzoate) (4). Compound 3 (200 mg) was dissolved in isopropanol/THF (v/v, 7:1, 10 mL), and cone. HCl (20 μL) was added into the solution. The mixture was stirred at 40° C. overnight. Concentration gave Boc-cleaved compound. The residue was again dissolved in DCM, followed by Boc₂O (100 mg) and Et₃N (776 μL). TLC showed the end of reaction. The organic phase was washed by Sat. NaHCO₃, brine and dried over Na₂SO₄. Purification through flash column chromatography gave 4 (95 mg, 60%).

tert-butyl 2,14-bis(benzyloxy)-1,3,13,15-tetrahydroxy-5,11-dioxo-7,8,9,11-tetrahydro-5H-dibenzo[g,i][1,5]dioxacycloundecin-8-ylcarbamate (5). n-Hexamine (120 mg) was dissolved in MeOH (3 mL) under N₂ atmosphere. Anhydride CuCl₂ (40 mg) was added to the solution and stirred at R.T. for 1 h. Compound 4 (50 mg) in MeOH (1 mL) was added dropwise into the coper solution. The mixture was stirred at R.T. for 3 h. 0.5M HCl was added to quench the reaction. The aqueous phase was extracted by ethyl acetate (X3). All the organic phases were combined and washed by 0.5M HCl (X2), brine and dried over Na₂SO₄. Purification through flash column chromatography gave 5 (26 mg, 65%) as yellowish oil.

tert-butyl 1,2,3,13,14,15-hexahydroxy-5,11-dioxo-7,8,9,11-tetrahydro-5H-dibenzo[g,i][1,5]dioxacycloundecin-8-ylcarbamate (Target 1). Compound 5 (150 mg) and Pd/C (69 mg) were dissolved in MeOH. The mixture was stirred under 1 atm H₂ at R.T. for 2.5 h. Concentration gave gray solid. The solid was stirred in DCM for 1 h and filtrated to give Target 1 (90 mg, 83%) as gray powder. Pre-HPLC gave as Target 1 (26 mg) white solid. ¹H NMR (400 MHz, DMSO-d₆): δ 8.00˜9.30 (m, 6H), 6.38 (s, 2H), 4.61 (m, 1H), 4.36 (m, 1H), 4.06 (m, 1H), 3.85 (m, 1H), 3.75 (m, 1H), 1.39 (s, 9H).

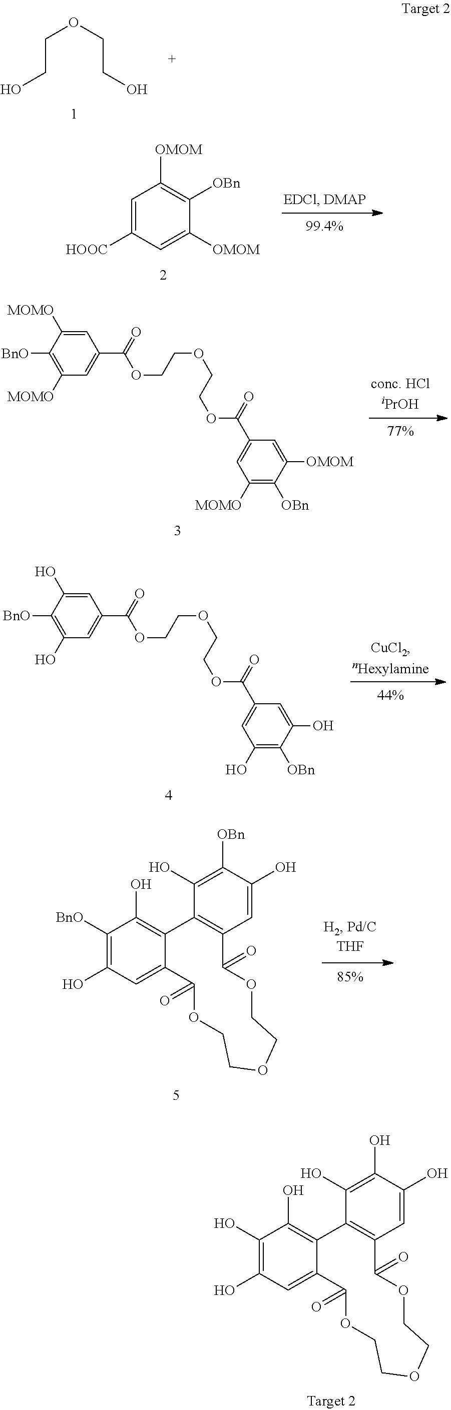

2,2′-oxybis(ethane-2,1-diyl) bis(4-(benzyloxy)-3,5-bis(methoxymethoxy)benzoate) (3). Compound 2 (400 mg, 1.15 mmol) was dissolved in DCM (5 mL). EDCI (440 mg, 2.3 mmol), DMAP (390 mg, 3.22 mmol) and Compound 1 (49 mg, 0.46 mmol) were added in sequence. The mixture was stirred at R.T. overnight. TLC showed the end of reaction. 1M H₃PO₄ was used to quench the reaction. The inorganic phase was extracted with DCM (X2). All the organic phases were combined and washed by 1M H₃PO₄, Sat. NaHCO₃, brine and dried over Na₂SO₄. Purification through flash column chromatography gave 3 (350 mg, 99.4%) as colorless oil.

2,2′-oxybis(ethane-2,1-diyl) bis(4-(benzyloxy)-3,5-dihydroxybenzoate) (4). Compound 3 (430 mg) was dissolved in isopropanol/THF (v/v, 7:1, 10 mL), cone. HCl (20 μL) was added into the solution. The mixture was stirred at 40° C. overnight. The organic phase was diluted by ethyl acetate and washed by Sat. NaHCO₃, brine and dried over Na₂SO₄. Purification through flash column chromatography gave 4 (256 mg, 77%).

2,16-bis(benzyloxy)-1,3,15,17-tetrahydroxy-7,8,10,11-tetrahydrodibenzo[i,k][1,4,7]trioxacyclotridecine-5,13-dione (5). n-Hexamine (137 mg) was dissolved in MeOH (4 mL) under N₂ atmosphere. Anhydride CuCl₂ (45 mg, 0.345 mmol) was added to the solution and stirred at R.T. for 1 h. Compound 4 (50 mg, 0.085 mmol) in MeOH (1.5 mL) was added drop wise into the coper solution. The mixture was stirred at R.T. for 3 h. 0.5M HCl was added to quench the reaction. The aqua phase was extracted by ethyl acetate (X3). All the organic phases were combined and washed by 0.5M HCl (X2), brine and dried over Na₂SO₄. Purification through flash column chromatography gave 5 (22 mg, 44%) as yellowish oil.

1,2,3,15,16,17-hexahydroxy-7,8,10,11-tetrahydrodibenzo[i,k][1,4,7]trioxacyclotridecine-5,13-dione (Target 2). Compound 5 (220 mg) and Pd/C (100 mg) were dissolved in MeOH. The mixture was stirred under 1 atm H₂ at R.T. for 2.5 h. Concentration gave gray solid. The solid was stirred in DCM for 1 h and filtrated to give Target 2 (130 mg, 85%) as gray powder. Pre-HPLC gave as Target 2 (40 mg) a white solid. ¹H NMR (400 MHz, DMSO-d₆): δ 8.30˜9.30 (bs, 6H), 6.72 (s, 2H), 4.31 (m, 2H), 3.86 (m, 2H), 3.66 (m, 2H), 3.55 (m, 2H), 1.39.

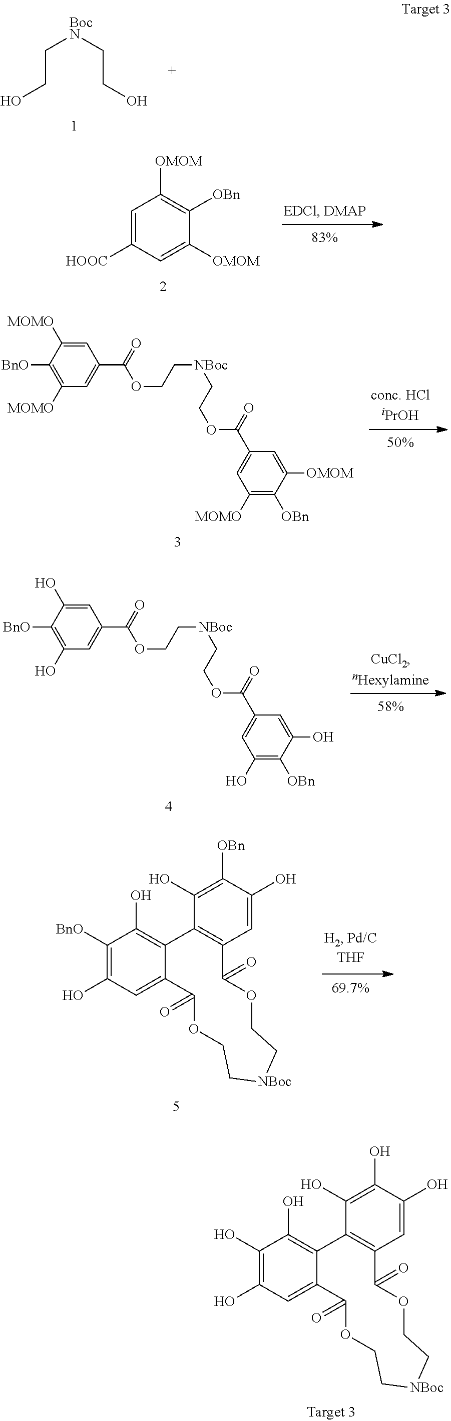

2,2′-(tert-butoxycarbonylazanediyl)bis(ethane-2,1-diyl) bis(4-(benzyloxy)-3,5-bis(methoxymethoxy)benzoate) (3). Compound 2 (400 mg, 1.15 mmol) was dissolved in DCM (20 mL). EDCI (440 mg, 2.3 mmol), DMAP (390 mg, 3.22 mmol) and Compound 1 (95 mg, 0.46 mmol) were added in sequence. The mixture was stirred at R.T. overnight. TLC showed the end of reaction. 1M H₃PO₄ was used to quench the reaction. The inorganic phase was extracted with DCM(X2). All the organic phases were combined and washed by 1M H₃PO₄, Sat. NaHCO₃, brine and dried over Na₂SO₄. Purification through flash column chromatography gave 3 (330 mg, 83%) as colorless oil.

2,2′-(tert-butoxycarbonylazanediyl)bis(ethane-2,1-diyl) bis(4-(benzyloxy)-3,5-dihydroxybenzoate) (4). Compound 3 (330 mg) was dissolved in isopropanol/THF (v/v, 7:1, 12 mL), cone. HCl (32 μL) was added into the solution. The mixture was stirred at 40° C. overnight. Concentration gave Boc-cleaved compound. The residue was again dissolved in DCM, followed by Boc2O (100 mg) and Et3N (776 μL). TLC showed the end of reaction. The organic phase was washed by Sat. NaHCO₃, brine and dried over Na₂SO₄. Purification through flash column chromatography gave 4 (90 mg, 50%).

tert-butyl 2,14-bis(benzyloxy)-1,3,13,15-tetrahydroxy-5,11-dioxo-7,8,9,11-tetrahydro-5H-dibenzo[g,i][1,5]dioxacycloundecin-8-ylcarbamate (5). n-Hexamine (1.2 g) was dissolved in MeOH (20 mL) under N₂ atmosphere. Anhydride CuCl₂ (398 mg) was added to the solution and stirred at R.T. for 1 h. Compound 4 (510 mg) in MeOH (10 mL) was added dropwise into the coper solution. The mixture was stirred at R.T. for 3 h. 0.5M HCl was added to quench the reaction. The aqueous phase was extracted by ethyl acetate (X3). All the organic phases were combined and washed by 0.5M HCl (X2), brine and dried over Na₂SO₄. Purification through flash column chromatography gave 5 (300 mg, 58%) as yellowish oil.

tert-butyl 1,2,3,13,14,15-hexahydroxy-5,11-dioxo-7,8,9,11-tetrahydro-5H-dibenzo[g,i][1,5]dioxacycloundecin-8-ylcarbamate (Target 3). Compound 5 (175 mg) and Pd/C (100 mg) were dissolved in MeOH. The mixture was stirred under 1 atm H₂ at R.T. for 2.5 h. Concentration gave gray solid. The solid was stirred in DCM for 1 h and filtrated to give Target 3 (90 mg, 69.7%) as gray powder. Pre-HPLC gave as Target 3 (40 mg) as a white solid. ¹H NMR (400 MHz, DMSO-d₆): δ 8.40˜8.90 (bs, 6H), 6.68 (s, 1H), 6.64 (s, 1H), 4.16 (m, 2H), 4.09 (m, 2H), 3.45 (m, 2H), 3.24 (m, 2H), 1.38 (s, 9H).

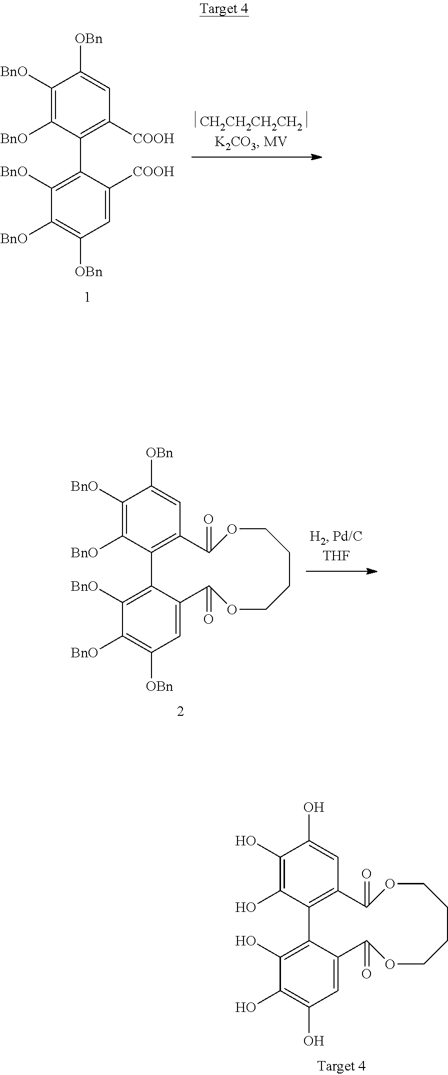 [SMILES: O=C(O)c1cc(OCc2ccccc2)c(OCc2ccccc2)c(OCc2ccccc2)c1-c1c(C(=O)O)cc(OCc2ccccc2)c(OCc2ccccc2)c1OCc1ccccc1 ; O=C1OCCCCOC(=O)c2cc(OCc3ccccc3)c(OCc3ccccc3)c(OCc3ccccc3)c2-c2c1cc(OCc1ccccc1)c(OCc1ccccc1)c2OCc1ccccc1 ; O=C1OCCCCOC(=O)c2cc(O)c(O)c(O)c2-c2c1cc(O)c(O)c2O]

1,2,3,14,15,16-Hexakis(benzyloxy)-7,8,9,10-tetrahydrodibenzo[h,j][1,6]dioxacyclododecine-5,12-dione (2). A suspension of compound 1 (50 mg), 1,4-diiodobutane (13.7 mg) and K₂CO₃ (31 mg) in CH₃CN (10 mL) was heated at 100° C. for 30 min under microware. Ten parallel batches were carried out and these were combined for work up. The combined mixture was concentrated under reduced pressure and the residue was purified by silica gel column chromatography (hexane:EtOAc=10:1 to 1:1) to afford 2b (190 mg, 36% yield) as yellow solid. ¹H NMR (400 MHz, CDCl₃): δ 6.90˜7.55 (m, 30H), 4.80˜5.30 (m, 12H), 4.50 (m, 2H), 4.15 (m, 2H), 2.05 (m, 2H), 1.85 (m, 2H).

Compound 2b (150 mg, 0.15 mmol) was dissolved in THF (25 mL), to the solution was added Pd/C (10%, 100 mg). The reaction mixture was stirred under 1 atm H₂ for 16 h then diluted with MeOH (25 mL). The mixture was filtered through a Celite pad and the cake was washed with MeOH (10 mL). The filtrate and elution was concentrated. The residue was triturated with MeOH (5 mL), filtered and dried under high vacuum to afford Taget 3 (50 mg, 85% yield) as dark powder. ¹H NMR (400 MHz, DMSO-d₆): δ 8.00˜8.30 (m, 6H), 6.43 (s, 2H), 4.21 (m, 2H), 3.88 (m, 2H), 1.85 (m, 2H), 1.75 (m, 2H).

### REFERENCES

1. Friedman, S. L., Sheppard, D., Duffield, J. S. & Violette, S. Therapy for fibrotic diseases: nearing the starting line. Science translational medicine 5, 167srl61 (2013). 2. Akhurst, R. J. & Hata, A. Targeting the TGFbeta signalling pathway in disease. Nat Rev Drug Discov 11, 790-811 (2012). 3. Finney, J., Moon, H. J., Ronnebaum, T., Lantz, M. & Mure, M. Human copper-dependent amine oxidases. Arch Biochem Biophys 546, 19-32 (2014). 4. Kagan, H. M. & Trackman, P. C Properties and function of lysyl oxidase. American journal of respiratory cell and molecular biology 5, 206-210 (1991). 5. Lee, M. J. et al. Pharmacokinetics of tea catechins after ingestion of green tea and (−)-epigallocatechin-3-gallate by humans: formation of different metabolites and individual variability. Cancer epidemiology, biomarkers & prevention: a publication of the American Association for Cancer Research, cosponsored by the American Society of Preventive Oncology 11, 1025-1032 (2002). 6. Liu, J. et al. Association of green tea consumption with mortality from all-cause, cardiovascular disease and cancer in a Chinese cohort of 165,000 adult men. European journal of epidemiology 31, 853-865 (2016). 7. Nagle, D. G., Ferreira, D. & Zhou, Y. D. Epigallocatechin-3-gallate (EGCG): chemical and biomedical perspectives. Phytochemistry 67, 1849-1855 (2006). 8. Thannickal, V. J., Zhou, Y., Gaggar, A. & Duncan, S. R. Fibrosis: ultimate and proximate causes. J Clin Invest 124, 4673-4677 (2014). 9. Bartram, U. & Speer, C P. The role of transforming growth factor beta in lung development and disease. Chest 125, 754-765 (2004). 10. Han, C. Y. et al. Hepcidin inhibits smad3 phosphorylation in hepatic stellate cells by impeding ferroportin-mediated regulation of Akt. Nat Commun 7, 13817 (2016). 11. Hu, C. et al. Regulation of TGFbeta1-mediated collagen formation by LOX-1: studies based on forced overexpression of TGFbeta1 in wild-type and lox-1 knock-out mouse cardiac fibroblasts. J Biol Chem 283, 10226-10231 (2008). 12. Sheppard, D. Transforming growth factor beta: a central modulator of pulmonary and airway inflammation and fibrosis. Proc Am Thorac Soc 3, 413-417 (2006). 13. Principe, D. R. et al. TGFbeta Signaling in the Pancreatic Tumor Microenvironment Promotes Fibrosis and Immune Evasion to Facilitate Tumorigenesis. Cancer Res 76, 2525-2539 (2016). 14. Travis, M. A. & Sheppard, D. TGF-beta activation and function in immunity. Annual review of immunology 32, 51-82 (2014). 15. Mordasky Markell, L., Perez-Lorenzo, R., Masiuk, K. E., Kennett, M. J. & Glick, A. B. Use of a TGFbeta type I receptor inhibitor in mouse skin carcinogenesis reveals a dual role for TGFbeta signaling in tumor promotion and progression. Carcinogenesis 31, 2127-2135 (2010). 16. Xi, Y. et al. Inhibition of epithelial-to-mesenchymal transition and pulmonary fibrosis by methacycline. American journal of respiratory cell and molecular biology 50, 51-60 (2014). 17. Peng, D. H. et al. ZEB1 induces LOXL2-mediated collagen stabilization and deposition in the extracellular matrix to drive lung cancer invasion and metastasis. Oncogene, doi:10.1038/onc.2016.358 (2016). 18. Peinado, H. et al. A molecular role for lysyl oxidase-like 2 enzyme in snail regulation and tumor progression. EMBO J 24, 3446-3458 (2005). 19. Erler, J. T. et al. Lysyl oxidase is essential for hypoxia-induced metastasis. Nature 440, 1222-1226 (2006). 20. Sethi, A., Mao, W., Wordinger, R. J. & Clark, A. F. Transforming growth factor-beta induces extracellular matrix protein cross-linking lysyl oxidase (LOX) genes in human trabecular meshwork cells. Invest Ophthalmol Vis Sci 52, 5240-5250 (2011). 21. Chen, C. Z. et al. The Scar-in-a-Jar: studying potential antifibrotic compounds from the epigenetic to extracellular level in a single well. Br J Pharmacol 158, 1196-1209 (2009). 22. Chen, C. Z. & Raghunath, M. Focus on collagen: in vitro systems to study fibrogenesis and antifibrosis state of the art. Fibrogenesis Tissue Repair 2, 7 (2009). 23. Yamauchi, M. & Sricholpech, M. Lysine post-translational modifications of collagen. Essays Biochem 52, 113-133 (2012). 24. Chen, Y. et al. Lysyl Hydroxylase 2 Is Secreted by Tumor Cells and Can Modify Collagen in the Extracellular Space. J Biol Chem 291, 25799-25808 (2016). 25. Dennler, S. et al. Direct binding of smad3 and smad4 to critical TGF beta-inducible elements in the promoter of human plasminogen activator inhibitor-type 1 gene. EMBO J 17, 3091-3100 (1998). 26. Zhang, H. M. et al. Research progress on the anticarcinogenic actions and mechanisms of ellagic acid. Cancer Biol Med 11, 92-100 (2014). 27. Stoner, G. D. et al. Pharmacokinetics of anthocyanins and ellagic acid in healthy volunteers fed freeze-dried black raspberries daily for 7 days. J Clin Pharmacol 45, 1153-1164 (2005). 28. Mertens-Talcott, S. U., Jilma-Stohlawetz, P., Rios, J., Hingorani, L. & Derendorf, H. Absorption, metabolism, and antioxidant effects of pomegranate (Punica granatum l.) polyphenols after ingestion of a standardized extract in healthy human volunteers. J Agric Food Chem 54, 8956-8961 (2006). 29. Seeram, N. P. et al. Pomegranate juice ellagitannin metabolites are present in human plasma and some persist in urine for up to 48 hours. JNutr 136, 2481-2485 (2006). 30. Lustosa, B. B. et al. Green tea (Cammellia sinensis) attenuates ventricular remodeling after experimental myocardial infarction. International journal of Cardiology 225, 147-153 (2016). 31. Kuriyama, S. et al. Green tea consumption and mortality due to cardiovascular disease, cancer, and all causes in Japan: the Ohsaki study. JAMA 296, 1255-1265 (2006). 32. Chapman, H. A. et al. Integrin alpha6beta4 identifies an adult distal lung epithelial population with regenerative potential in mice. J Clin Invest 121, 2855-2862 (2011). 33. Yamauchi, M. & Shiiba, M. Lysine hydroxylation and cross-linking of collagen. Methods Mol Biol 446, 95-108 (2008).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the formula: 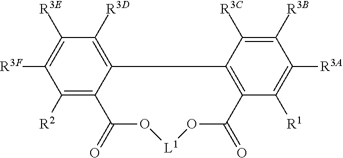
wherein,
R¹ is independently hydrogen, halogen, —CX¹₃, —CHX¹₂, —CH₂X¹, —OCX¹₃, —OCH₂X¹, —OCHX¹₂, —CN, —N₃, —SR^{1D}, —ONR^{1A}R^{1B}, —NHC(O)NR^{1A}R^{1B}, —NR^{1A}R^{1B}, —C(O)R^{1C}, —C(O)OR^{1C}, —C(O)NR^{1A}R^{1B}, —OR^{1D}, —NR^{1A}C(O)R^{1C}, —NR^{1A}C(O)OR^{1C}, —NR^{1A}OR^{1C}, R²⁰-substituted or unsubstituted C₁-C₄ alkyl, R²⁰-substituted or unsubstituted 2 to 4 membered heteroalkyl;
R²⁰ is independently oxo, halogen, —CX²⁰₃, —CHX²⁰₂, —CH₂X²⁰, —OCX²⁰₃, —OCH₂X²⁰, —OCHX²⁰₂, —CN, —N₃, —SR^{20H}, —ONR^{20E}R^{20F}, —NHC(O)NR^{20E}R^{20F}, —NR^{20E}R^{20F}, —C(O)R^{20G}, —C(O)OR^{20G}, —C(O)NR^{20E}R^{20F}, —OR^{20H}, —NR^{20E}C(O)R^{20G}, —NR^{20E}C(O)OR^{20G}, —NR^{20E}OR^{20G}, unsubstituted C₁-C₆ alkyl, or unsubstituted 2 to 6 membered heteroalkyl;
R² is independently hydrogen, halogen, —CX²₃, —CHX²₂, —CH₂X², —OCX²₃, —OCH₂X², —OCHX²₂, —CN, —N₃, —SR^{2D}, —ONR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, R²³-substituted or unsubstituted C₁-C₄ alkyl, or R²³-substituted or unsubstituted 2 to 4 membered heteroalkyl;
R²³ is independently oxo, halogen, —CX²³₃, —CHX²³₂, —CH₂X²³, —OCX²³₃, —OCH₂X²³, —OCHX²³₂, —CN, —N₃, —SR^{23H}, —ONR^{23E}R^{23F}, —NHC(O)NR^{23E}R^{23F}, —NR^{23E}R^{23F}, —C(O)R^{23G}, —C(O)OR^{23G}, —C(O)NR^{23E}R^{23F}, —OR^{23H}, —NR^{23E}C(O)R^{23G}, —NR^{23E}C(O)OR^{23G}, —NR^{23E}OR^{23G}, unsubstituted C₁-C₆ alkyl, or unsubstituted 2 to 6 membered heteroalkyl;
R^{3A}, R^{3B}, R^{3C}, R^{3D}, R^{3E}, and R^{3F} are independently —OH, —NH₂, hydrogen, —CX³₃, —CN, —N₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a R²⁶-substituted or unsubstituted 2 to 9 membered heteroalkylene, R²⁶-substituted C₂-C₉ alkylene, or R²⁶-substituted or unsubstituted C₄-C₆ cycloalkylene;
R²⁶ is independently —C(O)R^{26C}, —COOR^{26C}, —N₃, —CONR^{26A}R^{26B}, —NR^{26D}C(O)NR^{26A}R^{26B}, or —NR^{26A}C(O)OR^{26C};
R^{1A}, R^{1B}, R^{1C}, R^{1D}, R^{2A}, R^{2B}, R^{2C}, R^{2D}, R^{20E}, R^{20F}, R^{20G}, R^{20H}, R^{23E}, R^{23F}, R^{23G}, and R^{23H} are independently hydrogen, —CX₃, —CN, —COOH, —CONH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl;
R^{1A} and R^{1B} substituents bonded to the same nitrogen atom may optionally be joined to form an unsubstituted 3 to 6 membered heterocycloalkyl or unsubstituted 5 to 6 membered heteroaryl; R^{2A} and R^{2B} substituents bonded to the same nitrogen atom may optionally be joined to form an unsubstituted 3 to 6 membered heterocycloalkyl or unsubstituted 5 to 6 membered heteroaryl; R^{20E} and R^{20F} substituents bonded to the same nitrogen atom may optionally be joined to form an unsubstituted 3 to 6 membered heterocycloalkyl or unsubstituted 5 to 6 membered heteroaryl; R^{23E} and R^{23F} substituents bonded to the same nitrogen atom may optionally be joined to form an unsubstituted 3 to 6 membered heterocycloalkyl or unsubstituted 5 to 6 membered heteroaryl;
R^{26A} is independently hydrogen, —CF₃, —CN, —COOH, —CONH₂, R^{27A}-substituted or unsubstituted C₁-C₈ alkyl, R^{27A}-substituted or unsubstituted 2 to 8 membered heteroalkyl, R^{27A}-substituted or unsubstituted C₃-C₈ cycloalkyl, R^{27A}-substituted or unsubstituted 3 to 8 membered heterocycloalkyl, R^{27A}-substituted or unsubstituted C₆-C₁₀ aryl, or R^{27A}-substituted or unsubstituted 5 to 10 membered heteroaryl;
R^{26B} is independently hydrogen, —CF₃, —CN, —COOH, —CONH₂, R^{27B}-substituted or unsubstituted C₁-C₈ alkyl, R^{27B}-substituted or unsubstituted 2 to 8 membered heteroalkyl, R^{27B}-substituted or unsubstituted C₃-C₈ cycloalkyl, R^{27B}-substituted or unsubstituted 3 to 8 membered heterocycloalkyl, R^{27B}-substituted or unsubstituted C₆-C₁₀ aryl, or R^{27B}-substituted or unsubstituted 5 to 10 membered heteroaryl;
R^{26C} is independently R^{27C}-substituted or unsubstituted C₁-C₈ alkyl, hydrogen, oxo, halogen, —CF₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, R^{27C}-substituted or unsubstituted 2 to 8 membered heteroalkyl, R^{27C}-substituted or unsubstituted C₃-C₅ cycloalkyl, R^{27C}-substituted or unsubstituted 3 to 8 membered heterocycloalkyl, R^{27C}-substituted or unsubstituted C₆-C₁₀ aryl, or R^{27C}-substituted or unsubstituted 5 to 10 membered heteroaryl;
R^{26D} is independently hydrogen, —CF₃, —CN, —COOH, —CONH₂, R^{27D}-substituted or unsubstituted C₁-C₈ alkyl, R^{27D}-substituted or unsubstituted 2 to 8 membered heteroalkyl, R^{27D}-substituted or unsubstituted C₃-C₈ cycloalkyl, R^{27D}-substituted or unsubstituted 3 to 8 membered heterocycloalkyl, R^{27D}-substituted or unsubstituted C₆-C₁₀ aryl, or R^{27D}-substituted or unsubstituted 5 to 10 membered heteroaryl;
R^{26A} and R^{26B} substituents bonded to the same nitrogen atom may optionally be joined to form an R^{27A}-substituted or unsubstituted 3 to 8 membered heterocycloalkyl or R^{27A}-substituted or unsubstituted 5 to 10 membered heteroaryl;
R^{27A}, R^{27B}, R^{27C}, and R^{27D} are independently oxo, halogen, —CF₃, —CN, —N₃, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —OCF₃, —OCHF₂, —N₃, unsubstituted C₁-C₈ alkyl, unsubstituted 2 to 8 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl;
wherein at least one of the R^{3A}, R^{3B}, R^{3C}, R^{3D}, R^{3E}, or R^{3F} substituents are independently —OH; and
each X¹, X², X³, X²⁰, and X²³ is independently —F, —Cl, —Br, or —I.

2. The compound of claim 1, having the formula:

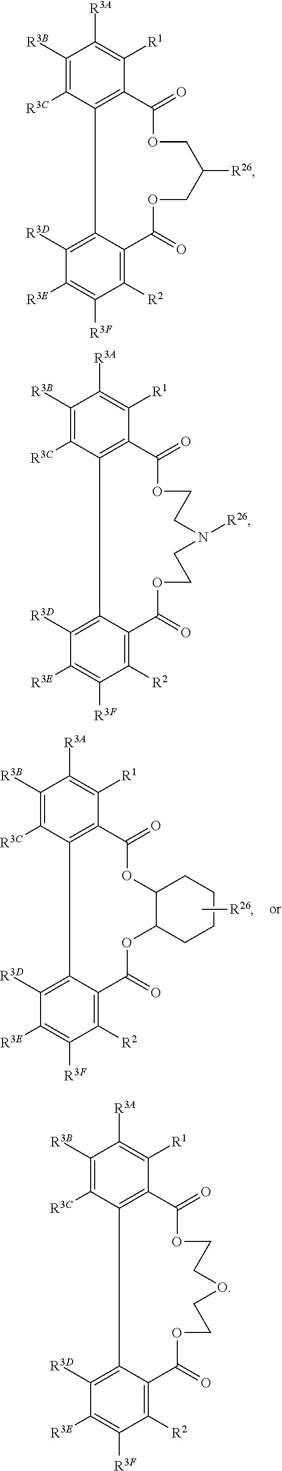

3. The compound of claim 1, wherein R^{3A} is —NH₂, —OH, or substituted or unsubstituted heteroalkyl.

4. (canceled)

5. The compound of claim 1, wherein R^{3B} is —NH₂, —OH, or substituted or unsubstituted heteroalkyl.

6. (canceled)

7. The compound of claim 1, wherein R^{3C} is —NH₂, —OH, or substituted or unsubstituted heteroalkyl.

8. (canceled)

9. The compound of claim 1, wherein R^{3D} is —NH₂, —OH, or substituted or unsubstituted heteroalkyl.

10. (canceled)

11. The compound of claim 1, wherein R^{3E} is —NH₂, —OH, or substituted or unsubstituted heteroalkyl.

12. (canceled)

13. The compound of claim 1, wherein R^{3F} is —NH₂, —OH, or substituted or unsubstituted heteroalkyl.

14. (canceled)

15. The compound of claim 1 having the formula:

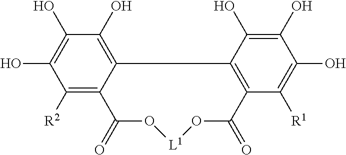

16. The compound of claim 15 having the formula:

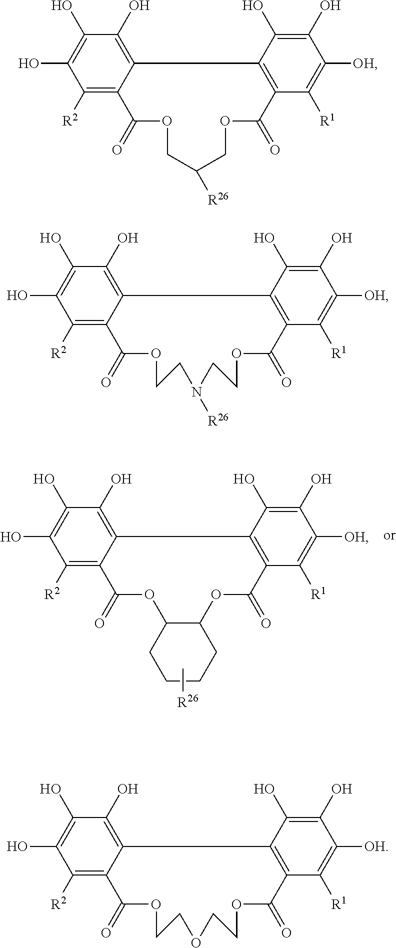

17. (canceled)

18. (canceled)

19. (canceled)

20. The compound of claim 1, wherein R²⁶ is independently —COOR^{26C}, —NR^{26A}C(O)OR^{26C}, or —C(O)R^{26C}.

21.-28. (canceled)

29. The compound of claim 1, wherein R¹ is independently hydrogen.

30. The compound of claim 1, wherein R² is independently hydrogen.

31. A pharmaceutical composition comprising the compound of claim 1 and a pharmaceutically acceptable excipient.

32. A method of reducing the level of activity of zinc finger protein Snail1 in a subject, said method comprising administering an effective amount of a compound of claim 1 to the subject.

33. A method of reducing the level of activity of Lysyl oxidase homolog 2 (LOXL2) in a subject, said method comprising administering an effective amount of a compound of claim 1 to the subject.

34. A method of inhibiting collagen cross-linking in a subject, said method comprising administering an effective amount of a compound of claim 1 to the subject.

35. A method of treating fibrosis, said method comprising administering to a subject in need thereof an effective amount of a compound of claim 1.

36. (canceled)

37. (canceled)

38. A method of treating cancer, said method comprising administering to a subject in need thereof an effective amount of a compound of claim 1.

39. (canceled)

40. A method of inhibiting Lysyl oxidase homolog 2 (LOXL2) protein activity and Transforming growth factor beta Receptor 1 (TGFβRI) protein activity in a cell, comprising contacting the LOXL2 protein with a LOXL2 generated TGFβRI inhibitor precursor; wherein the LOXL2 generated TGFβRI inhibitor precursor has the formula: 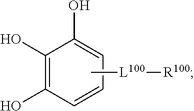
wherein
L¹⁰⁰ is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroaryl ene;
R¹⁰⁰ is independently halogen, —CX¹⁰⁰₃, —CHX¹⁰⁰₂, —CH₂X¹⁰⁰, —OCH₂X¹⁰⁰, —OCX¹⁰⁰₃, —OCHX¹⁰⁰₂, —N₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
X¹⁰⁰ is —F, —Cl, —Br, or —I.


SEQUENCE LISTING (19 sequences)

SEQ ID NO: 1 - protein, 274 aa, Homo sapiens
SNKVVMSGVKCSGTELSLAHCRHDGEDVACPQGGVQYGAGVACSETAPDLVLNAFMVQQTTYLEDRPMFMLQCAMEEKCLSASAAQTDPTTGYRRLLRFSSQIHNNGQSDFRPKNGRHAWIWHDCHRHYHSMEVFTHYDLLNLNGTKVAEGHKASFCLEDTECEGDIQKNYECANFGDQGITHGCWDNYRHDIDCQWVDITDVPPGDYLFQVVINPNFEVAESDYSNNIMKCRSRYDGHRINNYNCHIGGSFSEETEKKFEHFSGLLNNQLSPQ

SEQ ID NO: 2 - DNA, 24 bp, Homo sapiens
gacttccggcctaataatggccgc

SEQ ID NO: 3 - DNA, 24 bp, Homo sapiens
ggactggccattgttgtggatctg

SEQ ID NO: 4 - DNA, 24 bp, Homo sapiens
acaacatcatacgatgcaggagcc

SEQ ID NO: 5 - DNA, 24 bp, Homo sapiens
tggagtaatcggattctgcaacct

SEQ ID NO: 6 - DNA, 25 bp, Homo sapiens
acggaaaaacgttttgagcacttca

SEQ ID NO: 7 - DNA, 24 bp, Homo sapiens
ctcttcgctgaaggaaccacctat

SEQ ID NO: 8 - DNA, 16 bp, Mus musculus
ttcctggtggcagcgc

SEQ ID NO: 9 - DNA, 18 bp, Mus musculus
cggcacgctggaatgatc

SEQ ID NO: 10 - DNA, 21 bp, Artificial Sequence
  Synthetic polynucleotide
  misc_feature (1)..(1): Residue modified with 6-FAM
  misc_feature (21)..(21): Residue modified with BHQ
cccagtgcatggccgaactcg

SEQ ID NO: 11 - DNA, 20 bp, Mus musculus
tgtggagtgcccgatgtaca

SEQ ID NO: 12 - DNA, 20 bp, Mus musculus
tgtggagtgcccgatgtaca

SEQ ID NO: 13 - DNA, 28 bp, Artificial Sequence
  Synthetic polynucleotide
  misc_feature (1)..(1): Residue modified with 6-FAM
  misc_feature (28)..(28): Residue modified with BHQ
catcttagagcagtgccccagaggtcaa

SEQ ID NO: 14 - DNA, 17 bp, Mus musculus
tgcatcgcctgccattg

SEQ ID NO: 15 - DNA, 23 bp, Mus musculus
gacatttccacagtggaccttga

SEQ ID NO: 16 - DNA, 20 bp, Artificial Sequence
  Synthetic polynucleotide
  misc_feature (1)..(1): Residue modified with 6-FAM
  misc_feature (20)..(20): Residue modified with BHQ
ttggcccatggcaccctcca

SEQ ID NO: 17 - DNA, 20 bp, Mus musculus
caaaccgcatcaccacaaag

SEQ ID NO: 18 - DNA, 20 bp, Mus musculus
acgtggttcagccactcctc

SEQ ID NO: 19 - DNA, 23 bp, Artificial Sequence
  Synthetic polynucleotide
  misc_feature (1)..(1): Residue modified with 6-FAM
  misc_feature (23)..(23): Residue modified with BHQ
cacttgatcgcacccagaaggcc